(19) United States
(12) Patent Application Publication
(10) Pub. No.: US 2025/0064508 A1
(43) Pub. Date: Feb. 27, 2025

(54) METHODS AND DEVICES FOR REMOVING HEART VALVE THERAPY

(71) Applicant: AMX Technologies, LLC, Plymouth, MN (US)

(72) Inventors: Paul Sorajja, Plymouth, MN (US); David M. Costello, Plymouth, MN (US); Daniel P. Coyle, Plymouth, MN (US); Michael P. Brenzel, Plymouth, MN (US)

(73) Assignee: AMX Technologies, LLC, Plymouth, MN (US)

(21) Appl. No.: 18/727,061

(22) PCT Filed: Jan. 17, 2023
(86) PCT No.: PCT/US2023/060773

§ 371 Date: Jul. 5, 2024

Related U.S. Application Data
(60) Provisional application No. 63/266,799, filed on Jan. 14, 2022.
(60) Provisional application No. 63/362,507, filed on Apr. 5, 2022.
(60) Provisional application No. 63/368,588, filed on Jul. 15, 2022.

(51) Int. Cl. A61B 18/14; A61B 18/00

(52) U.S. Cl. CPC A61B 18/1492; A61B 2018/00369; A61B 2018/141

(57) ABSTRACT

A retrieval catheter and methods of use are described for removing a heart valve therapy such as a leaflet clip or artificial leaflet cord. The retrieval catheter can include a cutting element and a basket, piercing element, clamping mechanism, or similar grasping device. The method includes delivering a catheter to the region of the heart valve therapy and then manipulating the catheter and associated instruments to cut tissue as necessary and then remove the heart valve therapy and withdraw the catheter.

### RELATED APPLICATIONS

This application claims benefit of and priority to U.S. Provisional Application Ser. No. 63/266,799 filed Jan. 14, 2022 entitled Method and Device for Removing Chord and Tissue Surrounding Leaflet Apposition Therapy, U.S. Provisional Application Ser. No. 63/362,507 filed Apr. 5, 2022 entitled Heart Valve Therapy Clip Removal, and U.S. Provisional Application Ser. No. 63/368,588 filed Jul. 15, 2022 entitled Electrosurgical Devices and Methods, all of which are hereby incorporated herein by reference in their entireties.

### BACKGROUND OF THE INVENTION

Heart valve conditions can occur when the leaflets of a patient's valve are unable to fully close, which allows blood to regurgitate or abnormally flow backward. Referring to FIG. 1, regurgitation is especially common with the mitral valve 20 in which the mitral valve anterior leaflet 22 fails to properly coapt with the posterior leaflet 24. As the ventricles of the heart 10 contract, some blood moves from the left ventricle 14, back into the left atrium 12 instead of into the aorta 11. Similar regurgitation may also occur with the tricuspid valve 15, allowing blood to flow from the right ventricle 13 back into the right atrium 16.

A common treatment for valvular regurgitation is the use of treatment devices that appose or permanently connect the leaflets together. This heart valve therapy hardware may have been placed using surgical, transcatheter, or minimally-invasive means. For example, the hardware or therapy targeted for removal may be the MitraClip (Abbott Structural, Santa Clara, CA), the PASCAL device (Edwards Lifesciences, Irvine, CA), a suture placed surgically (e.g., Alfieri stitch), or similar heart valve therapy. Other heart valve therapy may be the result of techniques that have involved the leaflets as part of a therapeutic target, and the part or whole leaflet involvement requires removal. Other examples include chordal replacement technologies placed with either transcatheter methods or surgery to compensate for improper length, disruption, or mispositioning of existing chords. For purposes of the present specification, the phrase “heart valve therapy” or “heart valve therapy device” shall be defined as any devices and/or methods used for therapeutic treatment of a heart valve, such as leaflet clips, sutures, artificial chords, or any other devices or methods associated with the treatment of heart valves and associated leaflets.

FIG. 2 illustrates an example transcatheter delivery procedure for a valve clip 40 (e.g., a MitraClip) to treat a regurgitating mitral valve 20. A delivery catheter 41 is advanced through the right atrium 16, through the atrial septum 18, and into the left atrium 12. As seen best in FIG. 3, an inner portion of the catheter 41A including a valve clip 40, is advanced through the mitral valve 20 and into the left ventricle 14. In the present example, the leaflet clip 40 includes two outer arms 40A positioned underneath the leaflets 22, 24, and two inner arms 40B positioned vertically between the two leaflets 22, 24. As seen in FIG. 3, the catheter 41 includes control wires that can cause the outer arms 40A to close against the inner arms 40B to pinch or engage the tissue of the leaflets. Barbs or similar structures on the arms 40B help the leaflet clip 40 to anchor within the tissue of the leaflets 22, 24, as seen in FIG. 4. Finally, the catheter 41 is removed, as seen in FIG. 5. As seen in the top view of FIG. 6, the leaflet clip 40 is typically positioned near a center of the valve 20, preventing the center portion from opening and creating two smaller valve openings on either side of the clip 40. The smaller diameter of these openings typically allows the leaflets to better coapt and prevent regurgitation.

In some instances, these structures need to be removed in order to facilitate other valvular therapy, such as when there is recurrent or residual regurgitation that needs to be addressed. For example, the valve may require placement of other leaflet technologies, annuloplasty or rings, chordae or cords, positioning devices, or a replacement valve, many of which may not be usable with heart valve therapy previously performed.

In some instances, these therapies need to be removed from one or more attachment points on the leaflets, but not completely in order to facilitate other valvular therapy, leaving the structure in the heart but able to move it from the area of interest and apply desired therapy.

However, these heart valve therapies are typically removed via open heart surgery, which can be particularly traumatic for patients and presents a relatively high risk of complications. Therefore, what is needed is a less traumatic approach to removing heart valve therapy that presents a lower risk of complications.

### SUMMARY OF THE INVENTION

The present disclosure relates to systems and methods for removing heart valve therapies that have been used to position valve leaflets. This removal may be necessary when additional therapies for the treatment of valve disease are needed (e.g., different repair method, valve replacement), when the heart valve therapies have caused harm or the potential for harm to a patient (e.g., stenosis, infection), when the heart valve therapies have been deemed to not be of clinical benefit, or when there is a general desire to not have the therapy in place.

The present disclosure relates to systems and methods for removing heart valve therapies used to position leaflets, and this heart valve therapy may have been placed using surgical, transcatheter, or minimally-invasive means. In at least one embodiment, the hardware or therapy targeted for removal may be the MitraClip (Abbott Structural, Santa Clara, CA), the PASCAL device (Edwards Lifesciences, Irvine, CA), a suture placed surgically (e.g., Alfieri stitch), or similar positioning devices and techniques. In at least one embodiment, such positioning devices that need to be removed may be the result of techniques that have involved the leaflets as part of a therapeutic target, and the part or whole leaflet involvement requires removal. Examples of such devices are chordal replacement technologies placed with either transcatheter methods or surgery. In some instances, the cord or chords are not effective due to improper length, disruption, mispositioning, or defective prosthetic material.

A present method comprises a tool for cutting native valve tissue that has been attached to heart valve therapy with or without a capturing tool to hold the hardware to be removed while it is exteriorized from the human body. In at least one embodiment, the cutting method consists of an adjustable snare that envelops the heart valve therapy and can either cut the native tissue from the heart valve therapy mechanically, or by using a RF electrosurgical device that will heat tissue such that the electrosurgical cutting device's intracellular temperature rapidly reaches 100 degrees C., the intracellular contents undergo a liquid to gas conversion, massive volumetric expansion, and resulting vaporization. In at least one embodiment, the capturing tool is an adjustable basket, bag, or bin. This capturing tool can be used to cut, release, compress, modify, or fully retrieve the heart valve therapy from the human body.

In some embodiments, a method for removing previously placed heart valve therapy consists of a steerable catheter, which has been inserted into the patient using a transseptal, transatrial, or transventricular approach. The steerable catheter contains a delivery catheter that enables placement of the tools for cutting and for capturing the heart valve therapy.

In some embodiments, capture of the heart valve therapy is performed by insertion and embedding of a tool directly into, onto, and/or around the heart valve therapy. In this approach, the native tissue is cut from the heart valve therapy by the use of an electrosurgical cutting device (RF electrical or a similar device) or similar energy or force delivered from within the embedded tool. A basket or bag to capture the heart valve therapy may not be necessary for removal of the targeted material. Thus, in at least one embodiment, a cutting tool is used alone without the need for a capturing basket.

In at least one embodiment, a loop structure is pushed onto the tissue bridge, chordal implants, or method of fixation created by the heart valve therapy. The loop structure can be used to cut with either electrification or mechanical means. The loop structure may be circular, oval, or multi-segmented, and may completely or incompletely encapsulate the area for cutting and removal. The loop structure can be used to encircle the heart valve therapy and tissue for removal, followed by exteriorization.

In at least one embodiment, a tool is used to expand the heart valve therapy for removal. This expansion can be mechanical, electrical, pneumatic, hydraulic, or similar means in order to unfold or change the shape of heart valve therapy for its removal.

Elements of the tool can be fixated to the heart valve therapy to reduce the risk of embolization. This fixation can be accomplished by anchors that are straight, helical, barbed, or a combination of these approaches.

In at least one embodiment, a catheter, spacer, balloon, or other device could be used in conjunction with the removal device to manage the blood flow or regurgitation of the valve post removal of the heart valve therapy. This could be performed quickly if the removed heart valve therapy and basket could be retracted through the steerable catheter-then this sealing device could be delivered through the same delivery catheter.

A further embodiment of the present invention is directed to a removal system for a valve clip or similar heart valve therapy that may include a cutting and capture catheter and a snare catheter, both of which can be deployed from the same or separate delivery catheters. The snare catheter can be used to initially grasp and pull the valve clip distally (e.g., further into the left ventricle) to create tension or force on the leaflets. Next, the cutting loop and basket of the cutting and capture catheter can be placed over the valve clip so that the cutting loop is positioned on the proximal or atrial side of the heart valve therapy. Finally, the cutting loop can be activated to cut off the heart valve therapy device, and finally the clip can be captured by the basket and removed from the patient. This design enables cutting and capture of the heart valve therapy simultaneously as the cutting loop is pulled through the tissue and the capture basket is closed around the heart valve therapy device.

The snare catheter may include a snare loop having a circular shape or an oval saddle shape that creates an arc shape along each of its sides. The snare loop may have a plurality of teeth, a frictional coating, or can be composed of a coiled wire. The snare catheter may also include a distal tip with an opening in its sidewall and features to create friction with a snared valve clip, such as abrupt edges, ridges, grooves, or hooks.

The snare catheter may also include a handle configured to retract the snare loop into the snare catheter. The handle may include a mechanism to ensure tension is always applied to the clip, as well as limit the amount of force the user can apply to the tightened snare. The handle may include a locking mechanism to lock the snare in a desired retracted position.

The removal system may also include a guidewire passage through one or both of the cutting and capture catheter and a snare catheter. In a specific example, a guidewire passage may extend through the snare catheter and the basket of the cutting and capture catheter. The guidewire could traverse the length of the capture basket or pass through only the tip and then alongside the exterior surface of the basket.

The removal system may also include a chord dilator configured to at least partially block a distal opening of the delivery catheter and provide a relatively smooth transition which may provide less abrupt surfaces to “catch” on a chord or other feature of the valve.

The cutting and capture catheter may also include a stretchable capture basket that stretches from a longitudinally compressed configuration to a longitudinally stretched configuration once a valve clip is captured.

The removal system may also include a catheter that is configured to push on a valve clip from the atrial side of the valve to provide counter force. Hence, the cutting loop of the cutting and capture catheter may be better able to be positioned between the atrial side of the valve clip and the valve leaflets. The catheter may be configured solely for contact or may be configured to affirmatively engage or attach the valve clip.

The removal system may also include a snare catheter that includes a cutting element that allows it to engage tissue surrounding a valve clip or other heart valve therapy to facilitate capture and removal.

In some aspects, the techniques described herein relate to a snare catheter for use in medical treatment of a patient, including: an elongated sheath having a lumen and one or more openings at a distal portion of the elongated sheath into the lumen; an inner control member extending within the elongated sheath; a snare loop at a distal end of the inner control member and extending out of the one or more openings; and, electrical insulation configured to reduce transfer of electrical current between an electrical cutting device within a heart of a patient.

In some aspects, the techniques described herein relate to a snare catheter, wherein the electrical insulation is disposed over some or all of the snare loop.

In some aspects, the techniques described herein relate to a snare catheter, wherein the electrical insulation includes a coating layer, or tube including silicone, polyolefin, polyimide, dielectric material, PTFE, FEP.

In some aspects, the techniques described herein relate to a snare catheter, wherein the snare loop includes one or more wires including nitinol or stainless steel.

In some aspects, the techniques described herein relate to a snare catheter, wherein the elongated sheath further includes a distal tip member located at a distal end of the elongated sheath and forming the one or more openings; and wherein the electrical insulation further includes a layer, coating, or structural material.

In some aspects, the techniques described herein relate to a snare catheter, wherein the distal tip member includes rounded, curved, or blunted shapes around a perimeter of the one or more openings to reduce or limit damage to the electrical insulation.

In some aspects, the techniques described herein relate to a snare catheter, wherein the rounded, curved, or blunted shapes are formed by an insert connected to the distal tip member or are formed by the distal tip member.

In some aspects, the techniques described herein relate to a snare catheter, wherein the one or more openings is located on a side of the distal tip member relative to an axis of the elongated sheath.

In some aspects, the techniques described herein relate to a snare catheter for use in medical treatment of a patient, including: an elongated sheath having a lumen; an inner control member extending within the elongated sheath; a snare loop at a distal end of the inner control member; and, a distal tip member connected at a distal end of the elongated sheath, the distal tip member having a first opening and a second opening into the lumen of the elongated sheath; wherein the snare loop is positioned through the first opening and the second opening such that a structural feature in between the first opening and the second opening prevent the snare loop from being completely pulled into the lumen of the elongated sheath.

In some aspects, the techniques described herein relate to a snare catheter, wherein the structural feature of the distal tip member is a bar or a pin.

In some aspects, the techniques described herein relate to a snare catheter for use in medical treatment of a patient, including: an elongated sheath having a lumen and one or more openings at a distal portion of the elongated sheath into the lumen; an inner control member extending within the elongated sheath; and, a snare loop at a distal end of the inner control member and extending out of the one or more openings; wherein the snare loop is configured to limit deflection of a distal tip of the snare loop to an inclusive range of about 0 mm to about 10 mm when force is applied to the snare loop within an inclusive range of about 0 grams to about 3 grams.

In some aspects, the techniques described herein relate to a snare catheter, wherein the snare loop is configured to limit deflection of the distal tip of snare loop to an inclusive range of about 0 mm to about 5 mm when force is applied to the snare loop within an inclusive range of about 0 grams to about 2 grams.

In some aspects, the techniques described herein relate to a snare catheter, wherein the snare loop includes a solid shape memory wire having a diameter within an inclusive range of about 0.001 inch to 0.03 inch.

In some aspects, the techniques described herein relate to a tissue cutting catheter for use in medical treatment of a patient, including: an elongated catheter body; and, a cutting loop extending from a distal portion of the elongated catheter body; wherein the cutting loop includes one or more shape-memory wire segments and one or more electrodes including non-shape memory material.

In some aspects, the techniques described herein relate to a tissue cutting catheter, wherein the one or more electrodes include stainless steel.

In some aspects, the techniques described herein relate to a tissue cutting catheter, wherein the one or more electrodes include a tube or sleeve connected to the one or more shape-memory wire segments.

In some aspects, the techniques described herein relate to a tissue cutting catheter, wherein the one or more electrodes each include a segment of non-shape memory wire.

In some aspects, the techniques described herein relate to a tissue cutting catheter, wherein the one or more electrodes are radially larger than the one or more shape-memory wire segments.

In some aspects, the techniques described herein relate to a tissue cutting catheter, wherein the cutting loop is an open loop or a closed loop.

In some aspects, the techniques described herein relate to a tissue cutting catheter for use in medical treatment of a patient, including: an elongated catheter body; and, a cutting loop extending from a distal portion of the elongated catheter body; wherein the cutting loop includes primary loop segments and one or more electrodes; wherein the one or more electrodes have a diameter larger than the primary loop segments.

In some aspects, the techniques described herein relate to a tissue cutting catheter, wherein the one or more electrodes have a cylindrical, spherical, cubic, curved, and/or ridged shapes.

In some aspects, the techniques described herein relate to a tissue cutting catheter, wherein the one or more electrodes are disposed over part of some of the primary cutting loop segments; and wherein the one or more electrodes are positioned on top of, underneath, or adjacent to an insulating layer of the primary cutting loop segments.

In some aspects, the techniques described herein relate to a tissue cutting catheter, wherein the one or more electrodes extend fully around or partially around a circumference of one of the primary cutting loop segments.

In some aspects, the techniques described herein relate to a tissue cutting catheter, wherein the one or more electrodes are wire segments that are connected to adjacent segments of the primary cutting loop segments.

In some aspects, the techniques described herein relate to a tissue cutting catheter for use in medical treatment of a patient, including: an elongated catheter body; and, a cutting loop extending from a distal portion of the elongated catheter body; wherein the cutting loop includes one or more primary loop segments and an electrode; wherein the electrode includes a wire connected at a first location on the cutting loop and a second location on the cutting loop so as to create a space or gap with a portion of the cutting loop.

In some aspects, the techniques described herein relate to a tissue cutting catheter for use in medical treatment of a patient, including: an elongated catheter body; and, a cutting loop extending from a distal portion of the elongated catheter body; wherein the cutting loop includes a plurality of electrodes.

In some aspects, the techniques described herein relate to a tissue cutting catheter, wherein the plurality of electrodes include a first electrode located at a middle and distal-most location of the cutting loop.

In some aspects, the techniques described herein relate to a tissue cutting catheter, wherein the plurality of electrodes include a second electrode and a third electrode located on either side of the first electrode, and within a distal half of the cutting loop.

In some aspects, the techniques described herein relate to a tissue cutting catheter, wherein the plurality of electrodes includes a first electrode on a first lateral side of the cutting loop and a second electrode on a second lateral side of the cutting loop.

In some aspects, the techniques described herein relate to a capture basket catheter for use in medical treatment of a patient, including: an elongated catheter body; and, a capture basket at a distal portion of the elongated catheter body; wherein the capture basket is connected at a first distance from a distal end of the elongated catheter body and is connected at a second distance from the distal end of the catheter body, wherein the second distance is smaller than the first distance.

In some aspects, the techniques described herein relate to a capture basket catheter, wherein the capture basket is connected at the first distance by a cinching loop.

In some aspects, the techniques described herein relate to a capture basket catheter, wherein the capture basket is connected at the second distance between wires of the capture basket and an opening of the elongated catheter body.

In some aspects, the techniques described herein relate to a capture basket catheter, wherein the opening is located within a distal tip of the elongated catheter body.

In some aspects, the techniques described herein relate to a catheter delivery device for use in medical treatment of a patient, including: an elongated sheath having a sheath lumen therethrough; and, a sheath hub connected to a proximal end of the elongated sheath; wherein the sheath hub includes a plurality of ports opening into cavity; and wherein the cavity decreases in width distally; and wherein the cavity further including a funnel component at a distal end of the cavity and opening into the sheath lumen of the elongated sheath.

In some aspects, the techniques described herein relate to a chordae cutting device for use in medical treatment of a patient, including: an elongated catheter body having a catheter lumen and an opening at a distal portion of the elongated catheter body into the catheter lumen; a cutting element positioned within with catheter lumen and longitudinally movable therein; and, an elongated capture member having a distal end forming a curved shape when unconstrained and moveable to extend out of the opening by a user; wherein the curved shape is configured to at least partially surround one or more chordae within a heart and retract the chordae into the opening; and wherein the cutting element is distally movable against the chordae to cut the chordae within the catheter lumen.

In some aspects, the techniques described herein relate to a chordae cutting device, wherein the elongated capture member is positioned within a lumen of the cutting element.

In some aspects, the techniques described herein relate to a chordae cutting device, wherein the elongated capture member is positioned within the catheter lumen and outside of the cutting element.

In some aspects, the techniques described herein relate to a chordae cutting device, wherein the opening is located through a sidewall of the catheter body.

In some aspects, the techniques described herein relate to a chordae cutting device, wherein the curved shape is a loop, a spiral, a helical shape, a hook, or a plurality of loops terminating in a hook shape.

In some aspects, the techniques described herein relate to a chordae cutting device, wherein capture member includes one or more wires that have a diameter with an inclusive range of about 0.001 to 0.030 inch.

In some aspects, the techniques described herein relate to a chordae cutting device, wherein the curved shape has a working diameter within an inclusive range of about 0.15 inch to about 1.5 inches.

In some aspects, the techniques described herein relate to a chordae device, wherein the cutting element has a distal end forming a bias-cut edge, a plurality of points, a single point, or a step shape.

In some aspects, the techniques described herein relate to a chordae device, wherein the cutting element is a tubular shape or a solid shape.

In some aspects, the techniques described herein relate to a chordae device, wherein the cutting element is an electrode located on the capture member and configured to deliver electrical current to cut tissue.

In some aspects, the techniques described herein relate to a method of medical treatment for chordae, including: advancing an elongated catheter body into a heart; advancing a distal portion of an elongated capture member out of an opening of the elongated catheter body; allowing the distal portion of the elongated capture member to curve around one or more chordae; proximally moving the distal portion of the elongated capture member and part of the one or more chordae into the lumen of the elongated catheter body; and, actuating a cutting element within a lumen of the elongated capture member to cut the one or more chordae.

In some aspects, the techniques described herein relate to a method, wherein actuating a cutting element further included distally moving a cutting edge of the cutting element against the one or more chordae.

In some aspects, the techniques described herein relate to a method, wherein actuating a cutting element includes supplying RF power to an electrode on the elongated capture member.

In some aspects, the techniques described herein relate to a method, wherein allowing the distal portion of the elongated capture member to curve around the one or more chordae also including allowing the distal portion of the elongated capture member to curve around a heart valve clip.

In some aspects, the techniques described herein relate to a method of medical treatment for chordae, including: advancing an elongated catheter body into a heart; advancing a distal portion of an elongated capture member out of an opening of the elongated catheter body; allowing the distal portion of the elongated capture member to curve around one or more chordae and a heart valve clip; proximally moving the distal portion of the elongated capture member and part of the one or more chordae into a lumen of the elongated catheter body; and, advancing a separate cutting element into the heart and cutting the one or more captured chordae.

In some aspects, the techniques described herein relate to a method of medical treatment for chordae, including: advancing an elongated catheter body into a heart; advancing a distal portion of an elongated capture member out of an opening of the elongated catheter body; allowing the distal portion of the elongated capture member to curve around one or more chordae; and, actuating an electrode on the elongated capture member to cut the one or more chordae.

In some aspects, the techniques described herein relate to an RF power generator for medical treatment of a patient, including: an RF power generator housing including a processor, memory, and software code executable by the processor; and, a user interface controllable by the processor and software code; wherein the software code is configured to measure electrical signals of a plurality of electrodes on a cutting loop and then display whether each of the plurality of electrodes is in contact with tissue.

In some aspects, the techniques described herein relate to an RF power generator for medical treatment of a patient, including: an RF power generator housing including a processor, memory, and software code executable by the processor; and, a user interface controllable by the processor and software code; wherein the software code is configured to individually activate or deactivate each of a plurality of electrodes on a cutting loop.

In some aspects, the techniques described herein relate to a heart valve clip for treatment of a heart valve, including: a clip body; a leaflet connection mechanism configured to connect to leaflet tissue; and, a snare engagement feature including a channel, groove or enlarged structure at a bottom of the clip body.

In some aspects, the techniques described herein relate to a heart valve clip for treatment of a heart valve, including: a clip body; a leaflet connection mechanism configured to connect to leaflet tissue; and, electrodes configured to deliver electrical current when the clip body is supplied with electrical current.

In some aspects, the techniques described herein relate to a tissue cutting catheter for use in medical treatment of a patient, including: an elongated catheter body; and, a cutting loop extending from a distal portion of the elongated catheter body and including one or more electrodes; wherein the one or more electrodes each have a surface area within an inclusive range of about 0.003 square inch to about 0.006 square inch.

In some aspects, the techniques described herein relate to a tissue cutting catheter, wherein the one or more electrodes each have a surface area of about 0.00437506 square inch.

In some aspects, the techniques described herein relate to a tissue cutting catheter, wherein the one or more electrodes are connected to a power supply configured to supply RF power within an inclusive range of about 5 watts to 1600 watts.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 illustrates the anatomy of a heart.

FIG. 2 illustrates a side view of a procedure to implant a leaflet heart valve therapy device.

FIG. 3 illustrates a side view of a procedure to implant a leaflet heart valve therapy device.

FIG. 4 illustrates a side view of a procedure to implant a leaflet heart valve therapy device.

FIG. 5 illustrates a side view of a procedure to implant a leaflet heart valve therapy device.

FIG. 6 illustrates a top view of a procedure to implant a leaflet heart valve therapy device.

FIG. 7 illustrates a side view of a removal catheter according to the present invention.

FIG. 8 illustrates a side view of a removal catheter according to the present invention.

FIG. 9 illustrates a side view of a removal catheter according to the present invention.

FIG. 10 illustrates a side perspective view of a removal catheter according to the present invention.

FIG. 11 illustrates a side view of a removal catheter according to the present invention.

FIG. 12 illustrates a cross-sectional view of a removal catheter according to the present invention.

FIG. 13 illustrates an exploded view of a removal catheter according to the present invention.

FIG. 14 illustrates a perspective view of a cutting loop according to the present invention.

FIG. 15 illustrates a perspective view of a cutting loop according to the present invention.

FIG. 16 illustrates a perspective view of a cutting loop according to the present invention.

FIG. 17 illustrates a perspective view of a cutting loop according to the present invention.

FIG. 18 illustrates a perspective view of a cutting loop according to the present invention.

FIG. 19 illustrates a perspective view of a cutting loop according to the present invention.

FIG. 20 illustrates a top view of a cutting loop according to the present invention.

FIG. 21 illustrates a top view of a cutting loop according to the present invention.

FIG. 22 illustrates a perspective view of a cutting loop according to the present invention.

FIG. 23 illustrates a cross-sectional view of a cutting loop according to the present invention.

FIG. 24 illustrates a cross-sectional view of a cutting loop according to the present invention.

FIG. 25 illustrates a cross-sectional view of a cutting loop according to the present invention.

FIG. 26 illustrates a cross-sectional view of a cutting loop according to the present invention.

FIG. 27 illustrates a cross-sectional view of a cutting loop according to the present invention.

FIG. 28 illustrates a cross-sectional view of a cutting loop according to the present invention.

FIG. 29 illustrates a cross-sectional view of a cutting loop according to the present invention.

FIG. 30 illustrates a cross-sectional view of a cutting loop according to the present invention.

FIG. 31 illustrates a side view of a basket according to the present invention.

FIG. 32 illustrates a side view of a basket according to the present invention.

FIG. 33 illustrates a side view of a removal catheter according to the present invention.

FIG. 34 illustrates a side perspective view of a removal catheter according to the present invention.

FIG. 35 illustrates a side perspective view of a removal catheter according to the present invention.

FIG. 36 illustrates a side perspective view of a removal catheter according to the present invention.

FIG. 37 illustrates a side view of a removal catheter according to the present invention.

FIG. 38 illustrates a side view of a removal catheter according to the present invention.

FIG. 39 illustrates a side view of a removal catheter according to the present invention.

FIG. 40 illustrates a side view of a basket according to the present invention.

FIG. 41 illustrates a side view of a basket according to the present invention.

FIG. 42 illustrates a side view of a basket according to the present invention.

FIG. 43 illustrates a side view of a basket according to the present invention.

FIG. 44 illustrates a side view of a handle according to the present invention.

FIG. 45 illustrates a side view of a handle according to the present invention.

FIG. 46 illustrates a side view of a removal catheter according to the present invention.

FIG. 47 illustrates a side view of a removal catheter according to the present invention.

FIG. 48 illustrates a side view of a removal catheter according to the present invention.

FIG. 49 illustrates a side view of a removal catheter according to the present invention.

FIG. 50 illustrates a side view of a removal catheter according to the present invention.

FIG. 51 illustrates a side view of a removal catheter according to the present invention.

FIG. 52 illustrates a side view of a removal catheter according to the present invention.

FIG. 53 illustrates a side view of a removal catheter according to the present invention.

FIG. 54 illustrates a side view of a removal catheter and guide catheters according to the present invention.

FIG. 55 illustrates a side view of a removal catheter and guide catheters according to the present invention.

FIG. 56 illustrates a side view of a removal catheter procedure according to the present invention.

FIG. 57 illustrates a side view of a removal catheter procedure according to the present invention.

FIG. 58 illustrates a side view of a removal catheter procedure according to the present invention.

FIG. 59 illustrates a side view of a removal catheter procedure according to the present invention.

FIG. 60 illustrates a side view of a removal catheter procedure according to the present invention.

FIG. 61 illustrates a side view of a removal catheter procedure according to the present invention.

FIG. 62 illustrates a side view of a removal catheter procedure according to the present invention.

FIG. 63 illustrates a side view of a removal catheter procedure according to the present invention.

FIG. 64 illustrates a side view of a removal catheter procedure according to the present invention.

FIG. 65 illustrates a side view of a removal catheter procedure according to the present invention.

FIG. 66 illustrates a side view of a removal catheter procedure according to the present invention.

FIG. 67 illustrates a side view of a removal catheter procedure according to the present invention.

FIG. 68 illustrates a side view of a removal catheter procedure according to the present invention.

FIG. 69 illustrates a side view of a removal catheter procedure according to the present invention.

FIG. 70 illustrates a side view of a removal catheter procedure according to the present invention.

FIG. 71 illustrates a side view of a removal catheter procedure according to the present invention.

FIG. 72 illustrates a side view of a removal catheter procedure according to the present invention.

FIG. 73 illustrates a side view of a removal catheter procedure according to the present invention.

FIG. 74 illustrates a side view of a removal catheter procedure according to the present invention.

FIG. 75 illustrates a side view of a removal catheter procedure according to the present invention.

FIG. 76 illustrates a side view of a removal catheter procedure according to the present invention.

FIG. 77 illustrates a side view of a removal catheter procedure according to the present invention.

FIG. 78 illustrates a side view of a removal catheter procedure according to the present invention.

FIG. 79 illustrates a side view of a removal catheter procedure according to the present invention.

FIG. 80 illustrates a side view of a removal catheter procedure according to the present invention.

FIG. 81 illustrates a side view of a removal catheter procedure according to the present invention.

FIG. 82 illustrates a side view of a removal catheter procedure according to the present invention.

FIG. 83 illustrates a side view of a removal catheter procedure according to the present invention.

FIG. 84 illustrates a side view of a removal catheter procedure according to the present invention.

FIG. 85 illustrates a side view of a removal catheter procedure according to the present invention.

FIG. 86 illustrates a side view of a removal catheter procedure according to the present invention.

FIG. 87 illustrates a side view of a removal catheter procedure according to the present invention.

FIG. 88 illustrates a side view of a removal catheter procedure according to the present invention.

FIG. 89 illustrates a side view of a removal catheter procedure according to the present invention.

FIG. 90 illustrates a side view of a removal catheter procedure according to the present invention.

FIG. 91 illustrates a side view of a removal catheter procedure according to the present invention.

FIG. 92 illustrates a side view of a removal catheter procedure according to the present invention.

FIG. 93 illustrates a side view of a removal catheter procedure according to the present invention.

FIG. 94 illustrates a side view of a removal catheter procedure according to the present invention.

FIG. 95 illustrates a side view of a removal catheter procedure according to the present invention.

FIG. 96 illustrates a side view of a removal catheter procedure according to the present invention.

FIG. 97 illustrates a side view of a removal catheter procedure according to the present invention.

FIG. 98 illustrates a side view of a removal catheter procedure according to the present invention.

FIG. 99 illustrates a side view of a removal catheter procedure according to the present invention.

FIG. 100 illustrates a side view of a removal catheter procedure according to the present invention.

FIG. 101 illustrates a perspective view of a removal catheter apparatus according to the present invention.

FIG. 102 illustrates a perspective view of a removal catheter apparatus according to the present invention.

FIG. 103 illustrates a perspective view of a removal catheter apparatus according to the present invention.

FIG. 104 illustrates a perspective view of a removal catheter apparatus according to the present invention.

FIG. 105 illustrates a perspective view of a removal catheter apparatus according to the present invention.

FIG. 106 illustrates a perspective view of a removal catheter apparatus according to the present invention.

FIG. 107 illustrates a perspective view of example cuts to a valve.

FIG. 108 illustrates a perspective view of a removal catheter apparatus according to the present invention.

FIG. 109 illustrates a perspective view of a removal catheter apparatus according to the present invention.

FIGS. 110 and 111 illustrate views of a snare and removal system according to the present invention.

FIGS. 112 and 113 illustrate views of a basket tip according to the present invention.

FIGS. 114 and 115 illustrate views of a snare loop according to the present invention.

FIG. 116 illustrates a snare loop according to the present invention.

FIG. 117 illustrates a snare loop according to the present invention.

FIG. 118 illustrates a portion of a snare loop according to the present invention.

FIG. 119 illustrates a portion of a snare loop according to the present invention.

FIGS. 120 and 121 illustrate a snare catheter tip according to the present invention.

FIG. 122 illustrates a snare catheter tip according to the present invention.

FIG. 123 illustrates a snare catheter tip according to the present invention.

FIG. 124 illustrates a snare catheter tip according to the present invention.

FIGS. 125 and 126 illustrate a handle for a snare catheter according to the present invention.

FIGS. 127, 128, 129, and 130 illustrate a method of snaring and removing a valve clip according to the present invention.

FIGS. 131, 132, and 133 illustrate a removal system configured for access via a guidewire.

FIGS. 134, 135, 136, and 137 illustrate a chord dilator according to the present invention.

FIG. 138A illustrates a removal system with an alternate guidewire path.

FIG. 138B illustrates a removal system with an alternate guidewire path.

FIG. 139 illustrates a removal system with a pigtail wire on a distal end of its basket according to the present invention.

FIGS. 140 and 141 illustrate a stretchable basket according to the present invention.

FIG. 142 illustrates a method of applying force to a valve clip according to the present invention.

FIG. 143A illustrates an alternate basket shape according to the present invention.

FIG. 143B illustrates an alternate basket shape according to the present invention.

FIG. 144 illustrates an alternate removal system without a basket according to the present invention.

FIG. 145 illustrates an alternative removal system with a cutting element as part of the snare loop according to the present invention.

FIG. 146 illustrates an alternative removal system with a cutting element as part of the snare loop according to the present invention.

FIG. 147 illustrates a side view of a snare loop catheter according to the present invention.

FIG. 148 illustrates a front view of the snare loop catheter of FIG. 147 according to the present invention.

FIG. 149 illustrates a cross sectional view of the snare loop catheter of FIG. 147 according to the present invention.

FIG. 150 illustrates a side view of a handle of the snare loop catheter of FIG. 147 according to the present invention.

FIG. 151 illustrates a view of the distal tip of the snare loop catheter of FIG. 147 according to the present invention.

FIG. 152 illustrates a view of the distal tip of the snare loop catheter of FIG. 147 according to the present invention.

FIG. 153 illustrates a view of a distal tip of a snare loop catheter according to the present invention.

FIG. 154 illustrates a view of a distal tip of a snare loop catheter according to the present invention.

FIG. 155 illustrates a snare loop and inner control member according to the present invention.

FIG. 156 illustrates front view of a cutting loop catheter according to the present invention.

FIG. 157 illustrates a side view of the cutting loop catheter of FIG. 156 according to the present invention.

FIG. 158 illustrates a front view of a cutting loop catheter according to the present invention.

FIG. 159 illustrates a side view of the cutting loop catheter of FIG. 158 according to the present invention.

FIG. 160 illustrates a cross sectional view of an electrode according to the present invention.

FIG. 161 illustrates a cross sectional view of an electrode according to the present invention.

FIG. 162 illustrates a cross sectional view of an electrode according to the present invention.

FIG. 163 illustrates a cross sectional view of an electrode according to the present invention.

FIG. 164 illustrates a cross sectional view of an electrode according to the present invention.

FIG. 165 illustrates a cross sectional view of an electrode according to the present invention.

FIG. 166 illustrates a perspective view of an electrode according to the present invention.

FIG. 167 illustrates a front view of an electrode of a cutting loop according to the present invention.

FIG. 168 illustrates a side view of a cutting loop with multiple electrodes according to the present invention.

FIG. 169 illustrates a side view of the cutting loop of FIG. 168 according to the present invention.

FIG. 170 illustrates a top view of the cutting loop of FIG. 168 according to the present invention.

FIG. 171 illustrates a top view of a cutting loop with multiple electrodes according to the present invention.

FIG. 172 illustrates a side view of an open or hook shaped cutting loop according to the present invention.

FIG. 173 illustrates a side view of a capture basket catheter according to the present invention.

FIG. 174 illustrates a view of a distal tip of the capture basket catheter of FIG. 173 according to the present invention.

FIG. 175 illustrates a view of a distal tip of the capture basket catheter of FIG. 173 according to the present invention.

FIG. 176 illustrates a side view of a catheter hub according to the present invention.

FIG. 177 illustrates a cross sectional view of the catheter hub of FIG. 176 according to the present invention.

FIG. 178 illustrates a loading tool according to the present invention.

FIG. 179 illustrates the catheter hub of FIG. 176 according to the present invention.

FIG. 180 illustrates a heart valve therapy removal system according to the present invention.

FIG. 181 illustrates a side view of a chordae tool according to the present invention.

FIG. 182 illustrates a side view of a chordae tool according to the present invention.

FIG. 183 illustrates a side view of a chordae tool according to the present invention.

FIG. 184 illustrates a side view of a chordae tool according to the present invention.

FIG. 185 illustrates a side view of a chordae tool according to the present invention.

FIG. 186 illustrates a side view of a chordae tool according to the present invention.

FIG. 187 illustrates a side view of a cutting element for a chordae tool according to the present invention.

FIG. 188 illustrates a side view of a cutting element for a chordae tool according to the present invention.

FIG. 189 illustrates a side view of a cutting element for a chordae tool according to the present invention.

FIG. 190 illustrates a side view of a cutting element for a chordae tool according to the present invention.

FIG. 191 illustrates a method of using a chordae tool according to the present invention.

FIG. 192 illustrates a method of using a chordae tool according to the present invention.

FIG. 193 illustrates a method of using a chordae tool according to the present invention.

FIG. 194 illustrates a method of using a chordae tool according to the present invention.

FIG. 195 illustrates a method of using a chordae tool according to the present invention.

FIG. 196 illustrates a method of using a chordae tool according to the present invention.

FIG. 197 illustrates a method of using a chordae tool according to the present invention.

FIG. 198 illustrates a method of using a chordae tool according to the present invention.

FIG. 199 illustrates a method of using a chordae tool according to the present invention.

FIG. 200 illustrates a method of using a chordae tool according to the present invention.

FIG. 201 illustrates a power supply and interface according to the present invention.

FIG. 202 illustrates a power supply and interface according to the present invention.

FIG. 203 illustrates a heart valve clip according to the present invention.

FIG. 204 illustrates a heart valve clip according to the present invention.

FIG. 205 illustrates a heart valve clip according to the present invention.

FIG. 206 illustrates a heart valve clip according to the present invention.

FIG. 207 illustrates a heart valve clip according to the present invention.

FIG. 208 illustrates a heart valve clip according to the present invention.

### DETAILED DESCRIPTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

The present invention is generally directed to devices and methods for removing heart valve therapy devices via a transcatheter procedure. While current methods for removal of heart valve therapy devices require open heart surgery, the techniques and devices of the present invention utilize transcatheter devices and procedures which are less invasive and can provide better patient outcomes.

For purposes of the present application, the phrase “heart valve therapy” shall be defined as any devices and/or methods used for therapeutic treatment of a heart valve, such as leaflet clips, sutures, artificial chords, or any other devices or methods associated with the treatment of heart valves and associated leaflets. While specific embodiments may discuss or illustrate specific heart valve therapy devices or methods, such as heart valve leaflet clips, it should be understood that use with any heart valve therapy is specifically contemplated. Hence, none of the embodiments discussed in this specification should be limited solely to use with heart valve leaflet clips.

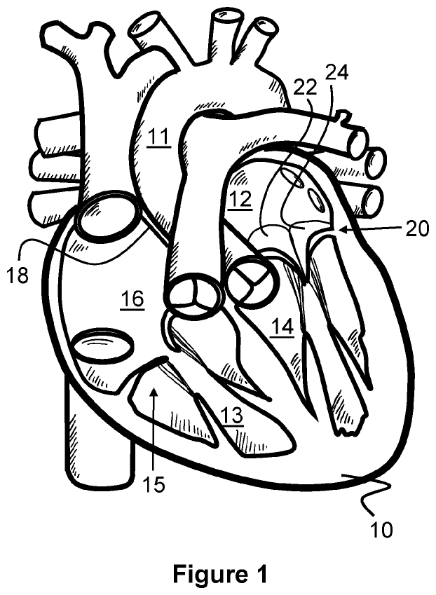

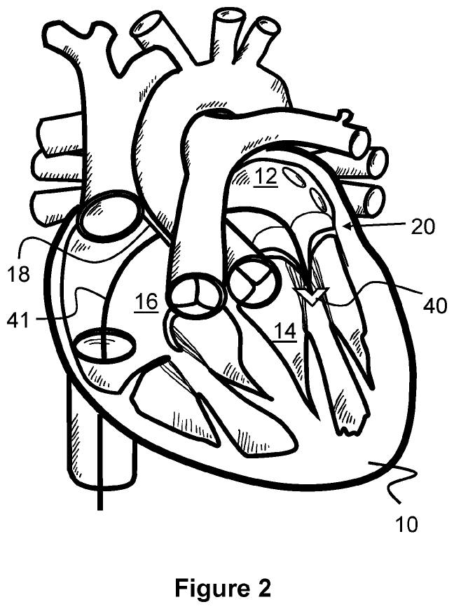

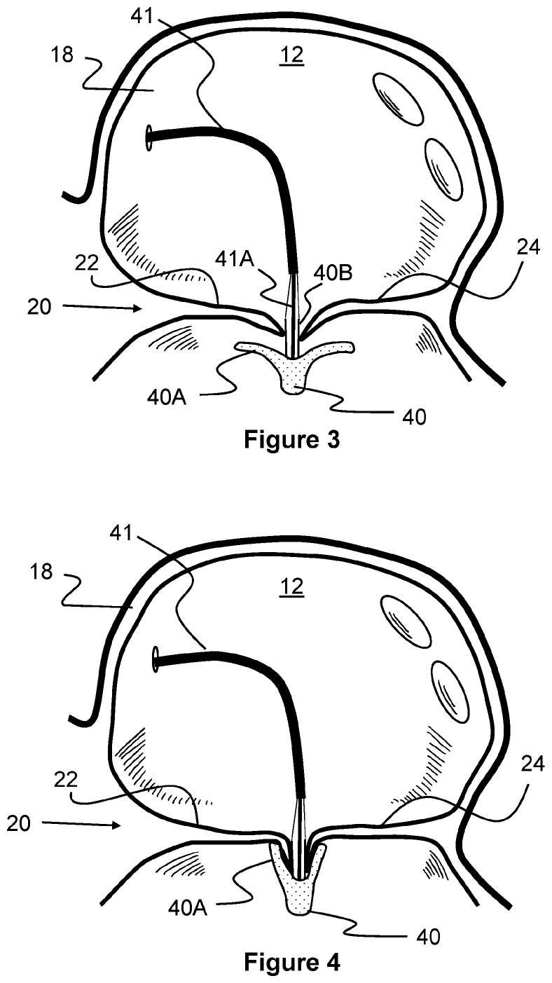

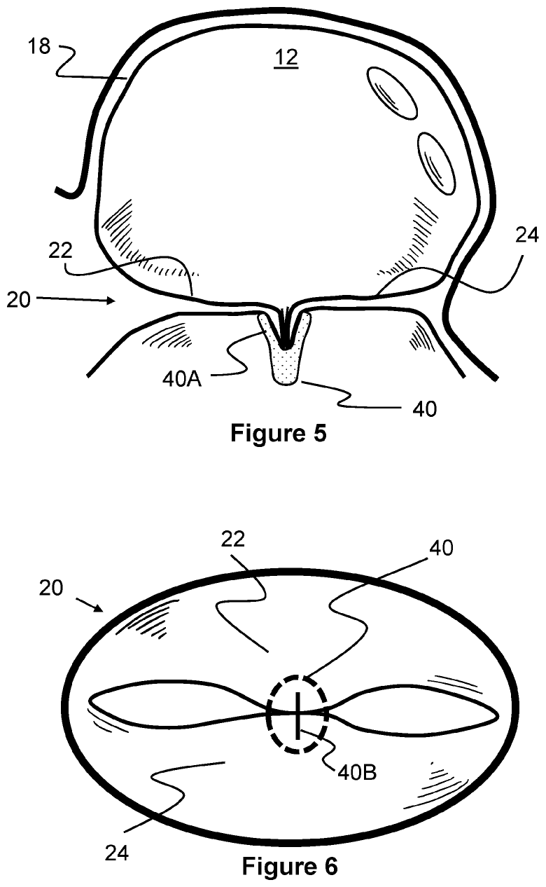

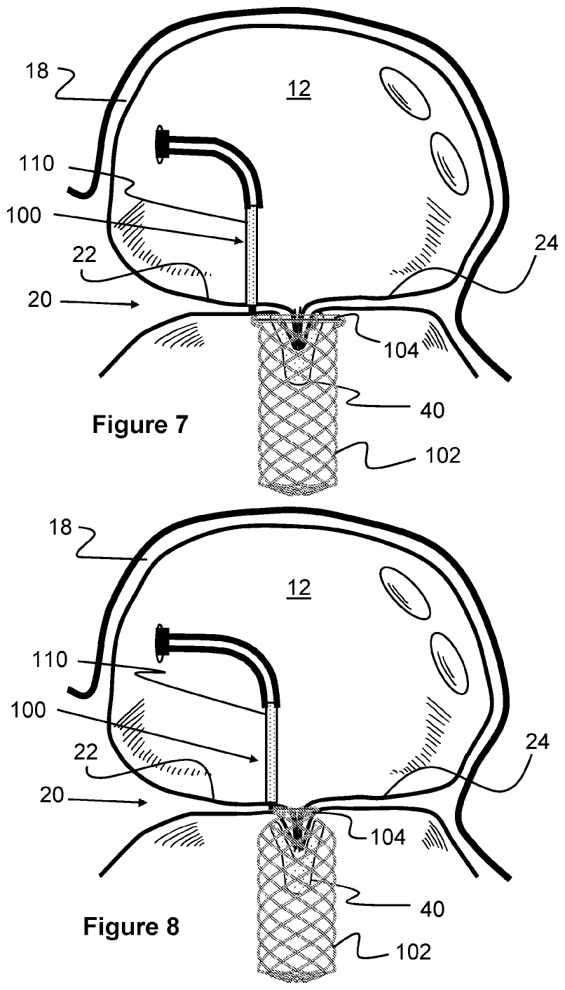

FIGS. 1-13 illustrate various aspects of one embodiment of a removal catheter 100 for removing a leaflet heart valve therapy device, such as a valve clip 40 or similar heart valve therapy device, according to the present invention. The removal catheter 100 generally includes an expandable capture basket 102 and a cutting element or cutting loop 104 that is disposed near a top opening of the basket 102. As seen in FIGS. 7 and 8, the basket 102 is placed over an implanted valve clip 40 so that a top of the basket 102 and the cutting loop 104 are positioned between the clip 40 and the leaflets 22, 24 on the leaflets 22, 24 atrial side. Next, the top opening of the basket 102 is closed or decreased in diameter and the cutting loop 104 is activated to cut the leaflet tissue surrounding the valve clip 40 (e.g., supplying radio frequency energy), freeing the clip 40 from the valve 20. Finally, the capture basket 102 containing the valve clip 40 is retracted and removed from the patient. Further details and variations of the removal catheter 100 are discussed below, followed by example approaches and methods of removal for various heart valves (e.g., a mitral valve 20 or a tricuspid valve 15).

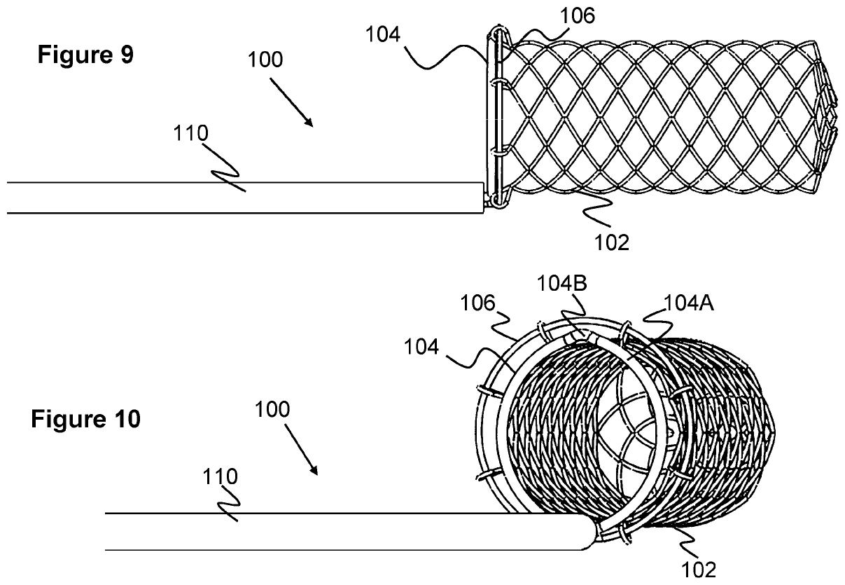

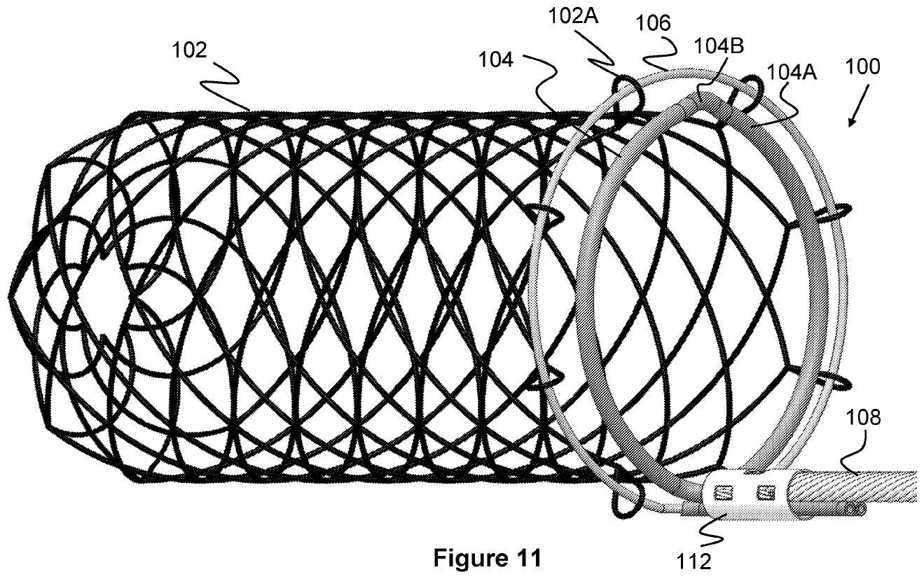

As best seen in FIGS. 9-11, the removal catheter 100 includes an inner control member 108 (seen best in FIG. 11) that is positioned within an outer tubular sheath 110. The inner control member 108 can be a solid wire or tube that extends between a distal end and a proximal end of the sheath 110. The basket 102 and cutting loop 104 are connected to a distal end of the inner control member 108 such that when the inner control member 108 is longitudinally or rotationally moved relative to outer tubular sheath 110, the basket 102 and cutting loop 104 are similarly moved.

Referring to FIG. 11, in one embodiment, a plurality of loops 102A are positioned around the circumference of the top opening of the basket 102 and a wire or cinching loop 106 is disposed through the loops 102A. As best seen in FIG. 13, the cinching loop 106 can be composed of a loop shaped wire (e.g., circular, oval, etc.) and an elongated straight portion 106A that that can be connected to the control member 108 via a connecting sleeve 112. The connecting sleeve 112 can be clamped, welded, applied with adhesive/epoxy, or any combination of the same to affix the sleeve 112 to the control member 108. Alternately, the cinching loop 106 can be connected to the control member 108 only via welding or adhesive.

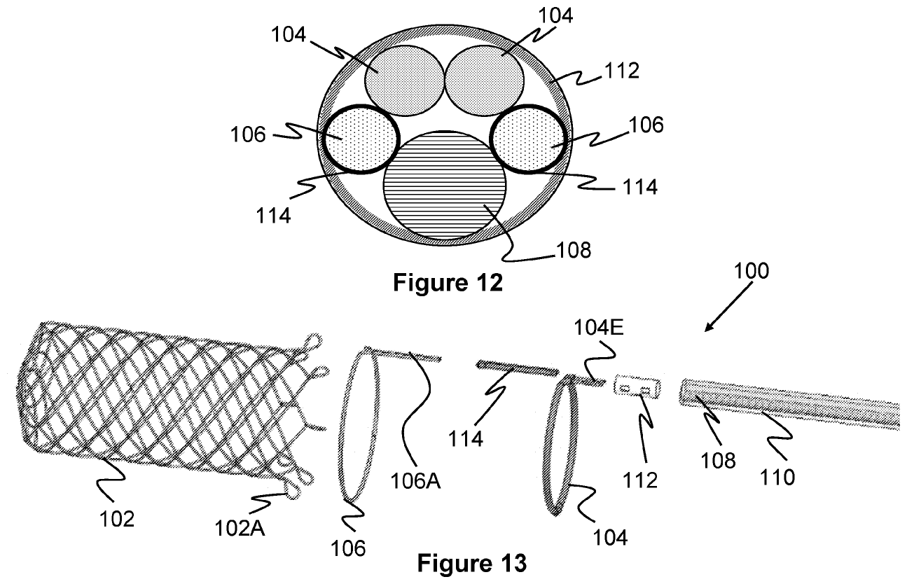

The cutting loop 104 can similarly be formed in a general loop shape (e.g., circular, oval, saddle shape, etc.) and can include an elongated straight portion 104E that can also be connected to the control member 108 via the connecting sleeve 112. In this respect, both of the elongated straight portions 106A and 104E are located within the connective sleeve 112, as seen in the cross-sectional view of FIG. 12.

In one embodiment, the cutting loop 104 cuts tissue when radio frequency energy is supplied to it. In one example, the RF power source is connected to a proximal end of the control member 108 which is composed of a conductive metal and therefore communicates the RF energy to its distal end and then into the attached cutting loop 104. To complete the RF energy circuit with the cutting loop 104, a second RF electrode can be connected to the RF power source and can be attached elsewhere to the patient via an electrode pad (a monopolar RF system), a second electrode can be included elsewhere on the removal catheter 100 (a bipolar RF system), or a second insulated wire can be included on the control member 108 (a bipolar RF system). Radiofrequency energy is a technical term established to describe high-frequency alternating electrical currents (with a frequency ranging from about 300 kHz to 3 MHz or more narrowly 100 kHz to 10 MHZ).

It may be desirable to isolate the RF energy circuit of the cutting loop 104 from both the cinching loop 106 and the basket 102 to prevent other tissue in the heart from being damaged. This can be achieved with the use of electrical insulation as specific locations on the device. For example, electrical wire insulation 114 can be placed over the elongated straight portion 106A (or optionally the entire cinching loop 106) to electrically isolate the cinching loop 106 from the RF current of the control member 108, as seen in the cross sectional view of FIG. 12 and the exploded view of FIG. 13.

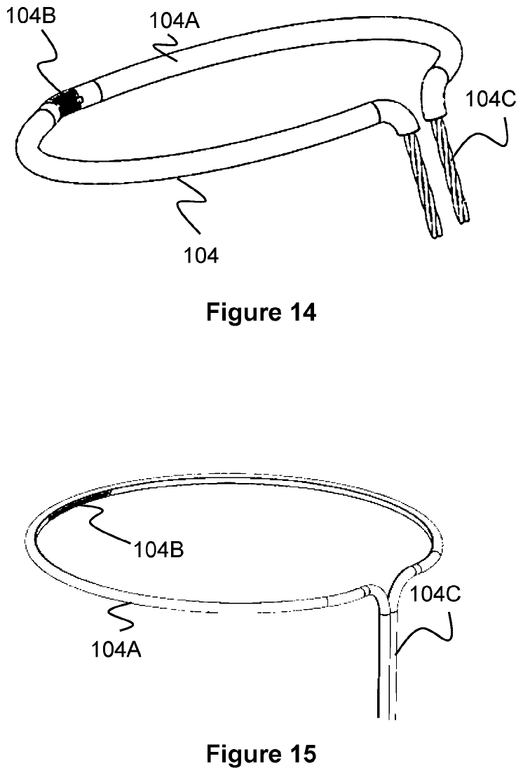

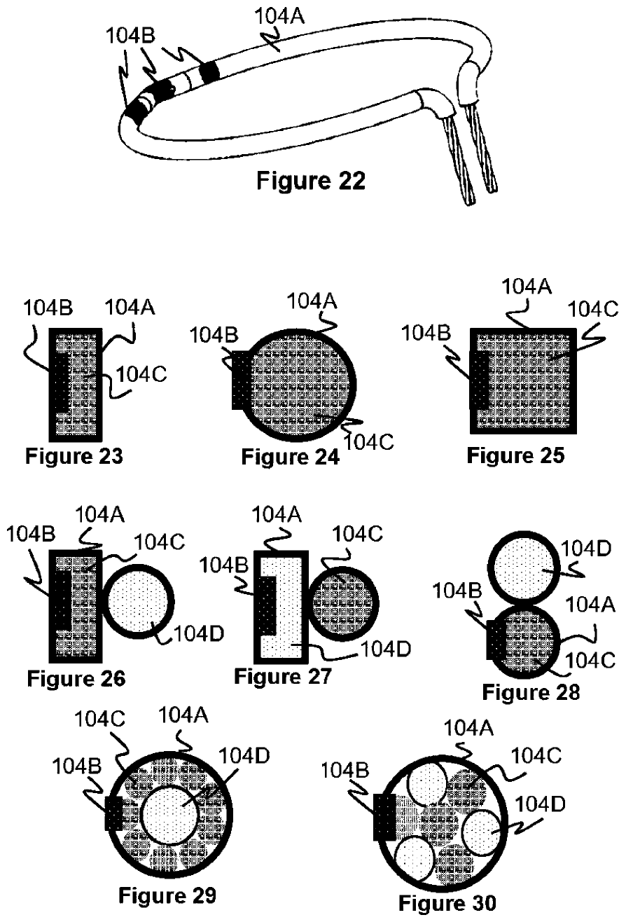

In other examples seen in FIGS. 14-30, the cutting loop 104 can have different structures, shapes, and electrical insulation to help reduce the risk of an uninsulated portion 104B (i.e., the portion that cuts the leaflet tissue) from contacting any portion of the cinching wire 106 or basket 102. For example, FIG. 14 illustrates a cutting loop 104 in which the uninsulated portion 104B of the wire is located opposite of the elongated straight portion 104E, adjacent to insulated portions 104A on each side. In this example, the uninsulated portion 104B can extend entirely around the circumference of the wire as seen in FIG. 21 or can only be exposed along the interior side of the loop 104 as seen in FIG. 20.

The uninsulated portion 104B may include only a single area in which the underlying wire 104C is exposed (e.g., between about 1 and 5 mm) as seen in FIG. 14 or can include a plurality of discrete uninsulated portions 104B (e.g., 2-10 portions 104B) of a relatively smaller length (e.g. between about 1 and 5 mm) as seen in FIG. 22.

In all cutting loop embodiments, the majority of the surface of the cutting loop 104 is insulated. To create the uninsulated portion 104B, the cutting loop insulation 104A can be selectively removed (for wires with existing insulation) to expose the cutting loop conduction wire 104C in a manner that will allow it to contact and deliver the RF cutting energy to the leaflet tissue bridge when it is in contact with tissue in proximity to the heart valve therapy. Alternately, the insulation 104A can be added (e.g., by dipping, spraying, or similar techniques) and the uninsulated portions 104B can be created by masking the intended areas prior to insulation application.

It will be understood that the uninsulated portion 104B can be oriented any number of ways, e.g., on the inner/outer surface of the cutting loop 104 as well as on the bottom (i.e., atrial) side of the loop 104.

The underlying wire 104C of the cutting loop 104 may be composed of a shape memory metal (e.g., Nitinol) or a similar conductive metal (e.g., stainless steel or copper). As seen in the cross-sectional views of FIGS. 23, 24, and 25, the underlying wire 104C can have a rectangular cross section, a circular cross section, a triangular cross section, and a square cross section, respectively.

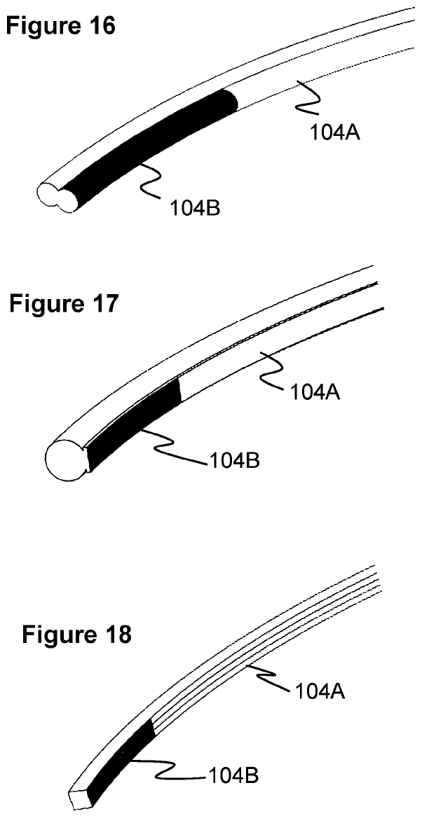

The cutting loop 104 may also be composed of one or more wires, such as a first wire 104C and a second wire 104D. Both wires can be composed of similar material (e.g., Nitinol, stainless steel, copper, silver, or similar materials), or each wire can be composed of a different material. For example, one wire 104C can be composed of a metal that better conducts current (e.g., stainless steel, silver, or copper) and the other wires 104D can be composed of a material that retains its shape between a compressed and expanded configuration (e.g., shape memory metal such as Nitinol). The multiple wires may be electrically isolated or insulated from each other or independently. Different cross sectional shapes can be further used with the same or different materials, as seen in FIGS. 16-18 and 26-28.

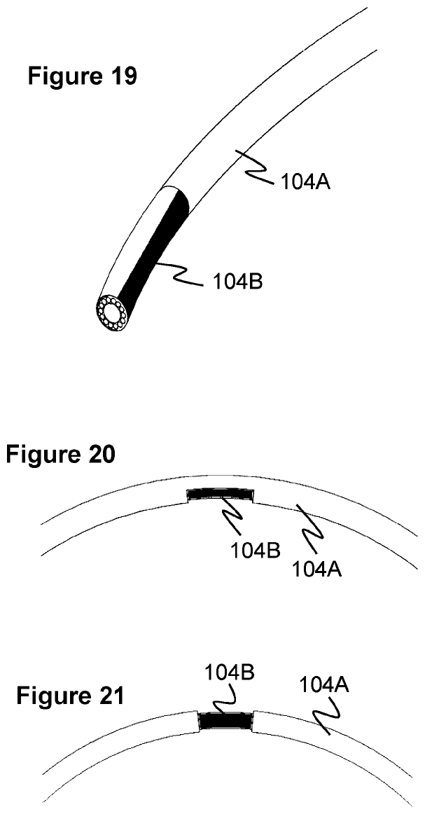

In another example, the cutting loop 104 may be composed of a single wire containing a plurality of strands of different wire materials. For example, FIGS. 19 and 29 illustrate a wire core 104D composed of a shape memory strand with a plurality of conductive strands 104C are located circumferentially around the core 104D. In another Example, FIG. 30 includes alternating shape memory strands 104D and conductive strands 104C. In this respect, the different strands may provide both desirable current conduction and the ability to expand to a predetermined loop shape from a compressed configuration. The multiple wires may be electrically isolated or insulated from each other or independently. Either of these two cable examples can have 2-49 or more strands within them.

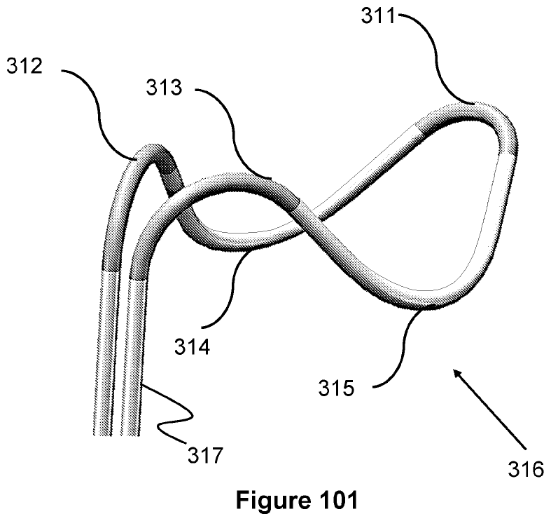

The cutting loop may have a variety of different shapes, structures, and electrical insulation patterns to facilitate tissue removal around the clip 104 that can, for example, provide additional length and/or a predetermined path or geometry. FIG. 101 illustrates one alternate example of a cutting loop 316 having a “saddle” or wave shape in which each side portion 314, 315 of the loop dips downward (i.e., in a proximal direction toward the catheter 100) and its free end 311 bends upwards (i.e., in a distal direction away from the catheter 100). Side portions 314 and 315 can be insulated and middle portion 311 and end portions 312 and 313 can be insulated. The middle portion 311 contacts or engages the tissue on one side of the loop 316, while end portions 312 and 313 contact or engage the tissue on the other side. The side portions 315 and 316 can bend outwards to increase the width of the loop 316, inwards to decrease the width of the loop 316, or can be relatively straight to maintain a uniform width of the loop 316 (i.e., circular or elliptical in shape).

Many different tissue engagement methods can be facilitated by to the cutting loop 316, such as end portions 312 and 313 can be electrically activated first in unison while the cutting loop 316 applies axial tension onto the tissue structure, effectively cutting the tissue in contact with those portions 312, 313 and partially freeing the leaflet clip 40. Next, the free end portion 311 can be activated to excise the tissue adjunct to it and completing the excision of the leaflet clip 40 from the leaflets. Alternately, all three portions 311, 312, and 313 can be activated at the same time. Axial tension on the loop 316 can be applied before, during, or intermittently to control the engagement of the loop 316. This embodiment illustrates three uninsulated cutting areas or portions 311, 312, and 313, however there may be any number of cutting elements (e.g., from 1-100), including the entire loop 316 as being one continuous, uninsulated cutting member.

Including additional length along the side portions 314 and 315 can accommodate other tissue structures present around the leaflet clip 40. The extra length of the side portions 314 and 315 can also be deformable such that when tension is applied by the elongated straight portions 317, the side portions 314 and 315 will straighten and cause the loop 316 to elongated to an approximate axial configuration. During this tension and elongation, the axial distance between the free end portion 311 and the proximal end portions 312, 313 is increased, accommodating a greater variation in both diameter and approach angle to the clip. Any such nonlinear path could also accomplish this and are hence considered in this disclosure, but for sake of brevity are not shown herein.

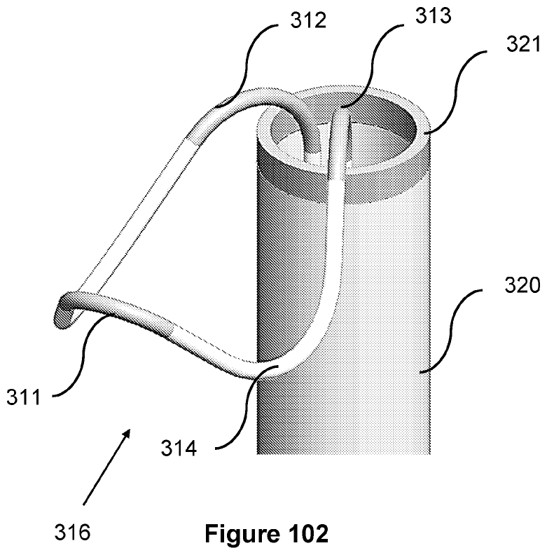

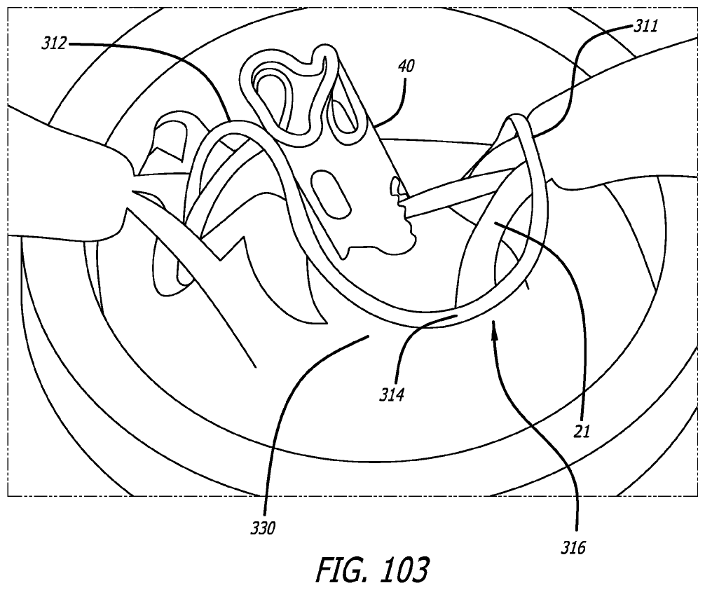

FIG. 102 illustrates a delivery mechanism with a previously described cutting loop 316 and an outer tubular sheath 320 that is generally similar to that of previously described sheath 110. However, the outer tubular sheath 320 further includes a sheath cutting portion 321 that is disposed at and circumferentially around the distal end of the sheath 320. This sheath cutting portion 321 can be of similar construction and characteristics as previously described uninsulated portions of the prior cutting loops and can be similarly electrically activated to provide facilitate additional areas of tissue that can be cut. This outer tubular sheath 320 can be used in conjunction with any of the other apparatuses disclosed and in a method that best facilitates the leaflet clip removal procedure. Again, all of the uninsulated cutting portions 311, 312, 313, and 321 can be activated individually at different times or all together at the same time.

FIG. 103 illustrates the embodiment of FIG. 102 within a heart valve tissue model 330, with chordae 21 and a leaflet positioning clip 40. This figure illustrates one example of how the apparatuses 320 and 316 engage tissue on all sides of the clip 40 in a manner that is positioned to sever the attaching tissue structures from the clip apparatus 40.

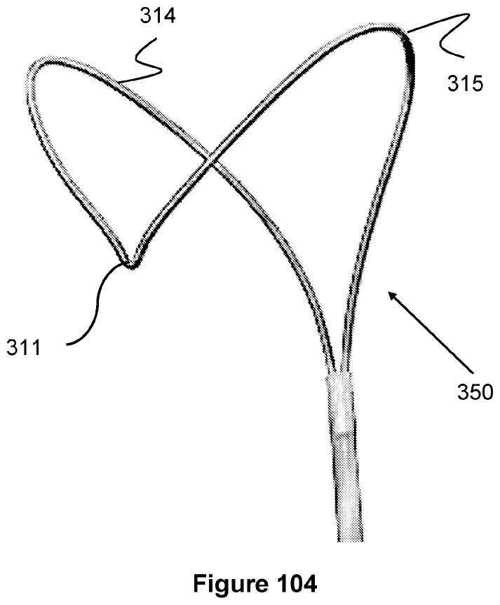

Another example embodiment of a cutting loop 350 can be seen in FIG. 104, in which each side loop portion 314, 315 bends upward (i.e., in a distal direction) and its free end bends downward (i.e., in a proximal direction). Additionally, the side loop portions 314, 315 are shown bending laterally outward, increasing the width of the loop 350. The loop 350 can have a variety of different insulated and uninsulated portions, such as those described in FIGS. 101-103 (i.e., several discrete uninsulated portions or the entire loop being uninsulated).

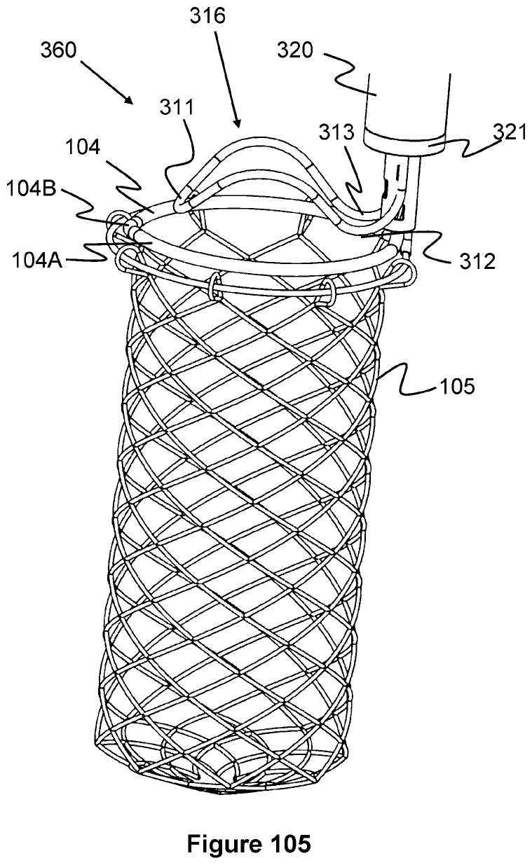

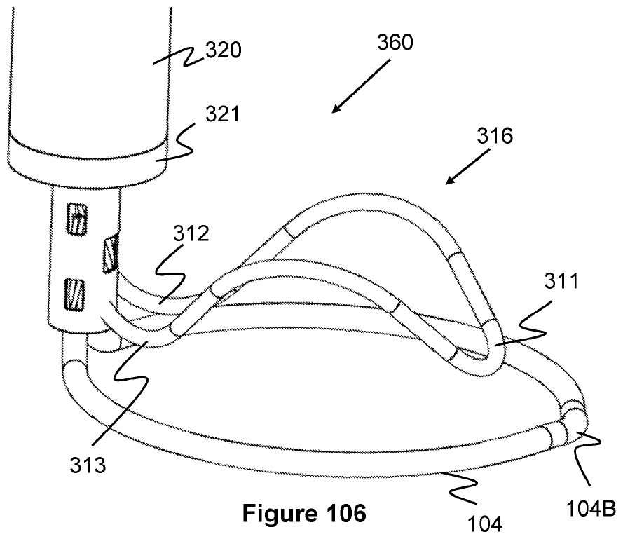

FIGS. 105 and 106 illustrate another embodiment of a removal device 360 that is generally similar to the removal device 100 but further includes a first cutting loop 104 and a second cutting loop 316. In some instances, it may be difficult for the physician to visualize exactly what tissue should be cut to completely remove a heart therapy device. Two or more loops may allow for a first series of cuts to the valve tissue and then one or more second cuts (e.g., via cinching the first cutting loop) to completely remove the heart therapy without the need for dramatic repositioning of the loops. In contrast, a single cutting loop may need to be moved, longitudinally repositioned, and/or rotated to fully cut out the heart therapy.

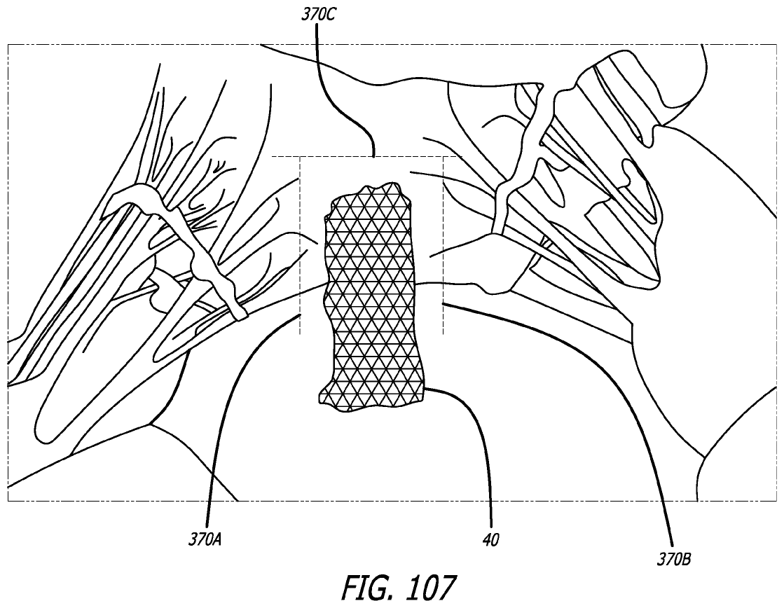

In the present example, the first cutting loop 104 has a somewhat larger diameter (e.g., similar to the opening of the basket 104) and the second cutting loop 316 has a diameter that is smaller than the first cutting loop 104 and that is positioned further away from the basket 104. Hence, the second loop 316 may be placed against the valve leaflets and/or chords (e.g., cut 370A through the antero-lateral chords and cut 370B through the postero-medial chords in FIG. 107) and the cutting portions 311, 313, and 313 can be activated to perform the first series of cuts to the tissue. This first series of cuts may not cut all of the tissue, however the cutting portion 104B of first cutting loop 104 can cinched and then activated to perform one or more second cuts to completely remove any remaining tissue from the heart therapy device 40 (e.g., along cut 370C on the atrial side of the clip 40 in FIG. 107).

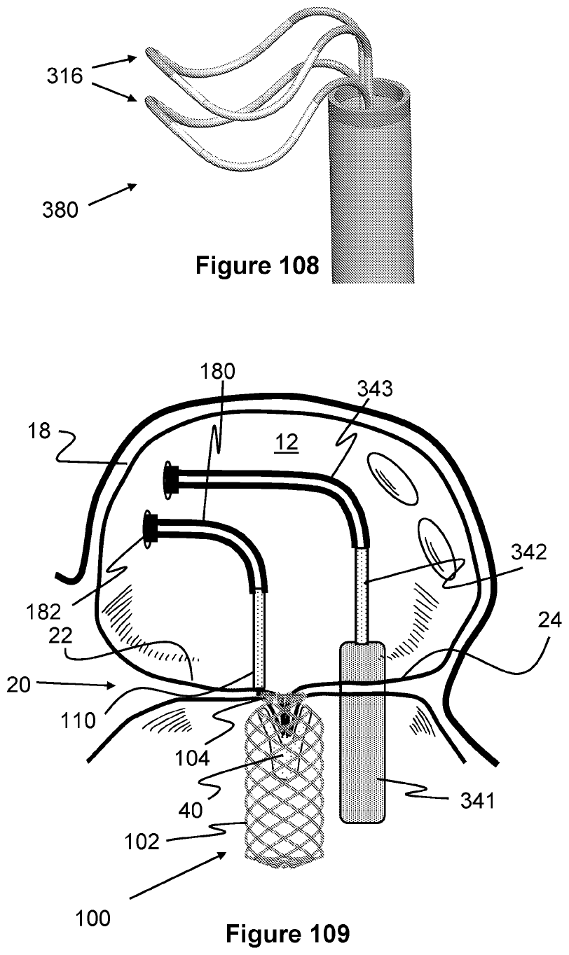

While specific embodiments of the cutting loops 104 and 316 are shown in FIGS. 105 and 106, any combination of any of the loops described in this specification can be used in this manner. For example, FIG. 108 illustrates an embodiment 180 with two loops 316 of similar shape and configuration. Hence, either of the loops may have different numbers and patterns of cutting portions and may activate those cutting portions all simultaneously or at different times/patterns. In one example, both of the cutting loops 104 and 316 are connected to the same electrical circuit (e.g., the inner control member 108). Alternately, each loop 104, 316 (or alternately each set of cutting portions) may have its own electrical circuit (e.g., individual conducting wires) that allows for independent activation from the other cutting loop. Additionally, three or four cutting loops may alternately be used. The cutting loops may all be connected to the same removal catheter or one or more loops can be connected to a catheter separate from other cutting loops and/or the basket 104. In some embodiments one or more cutting loops may be located on the ventricular side of the valve while one or more cutting loops may be located on the atrial side of the valve.

If the cinching loop 106 has an insulation coating entirely along its length, the cutting loop 104 may be located directly on top of the cinching loop 106, contacting the loop. The cutting loop 104 may also be longitudinally spaced apart from the cinching loop 106, such as between about 0 mm and about 15 mm.

Preferably, the inner control member 108 (seen best in FIGS. 11-13) is flexible enough to navigate through the vasculature while having enough column strength to push the basket 102 and cutting loop 104 out of the outer tubular sheath 110, be capable of efficiently delivering RF energy from the proximal handle to the cutting loop, be insulated to prevent current leakage to the bloodstream, and have good torque response so the user can rotate the basket and loop when it is deployed in and around the valve.

In a preferred embodiment the inner control member 108 consists of an inner control stylet that is joined or welded to a more flexible inner control cable, which is then joined to the cutting loop conduction wire tails using a distal coupler. in a preferred embodiment the inner control stylet, inner control cable, cutting loop conduction wire, and distal coupler are the same material (e.g., steel alloy) to enable a strong weld joint and efficient current delivery throughout. The inner control cable could be a laser cut tube, a stranded cable, a stranded cable tube, a coil, or a combination of these. In another embodiment, the inner control cable may extend from the proximal handle to the cutting loop 104, and eliminate the need for the inner control stylet.

In an alternate embodiment, the inner control member 108 can be two separate wires; one of which connects to the cinching loop 106 and the other that connects to the cutting loop 104. In the case of both inner control members being disposed in the same single lumen of the outer tubular sheath 110, the basket 102 may be deployed first by advancing the inner basket control member distally until the basket cinching loop 106 is fully exposed. Then, the inner cutting loop control member can be advanced distally to deploy the cutting loop 104. Each of the loops can be rotated, advanced, or retracted by their respective control members. This provides the operator with more degrees of freedom. The heart valve therapy may be first captured or encircled by the cutting loop 104, and then the basket cinching loop 106 and basket 102 can follow. The cutting loop 104 can then be closed onto the leaflet tissue bridge by retracting the inner cutting loop control member. Once the cutting loop is closed on the tissue bridge, one of two steps can be taken: 1) the basket cinching wire 104 and basket 102 can then be closed by retracting the inner basket control member proximally or 2) if the cutting loop 104 is unable to get to the base of the heart valve therapy, RF cutting energy can be applied to cut down one side of the device to get to the base of the clip 40; then the basket 102 can be closed. Once both loops are properly closed on the tissue on the atrial side of the heart valve therapy, the inner cutting loop control member is energized with RF power as it is retracted proximally into the outer delivery sheath 110. The inner cutting loop control member delivers the cutting energy to only the cutting element through the cutting loop 104.

The aforementioned inner control members can alternately be disposed in separate outer tubular sheaths or separate lumens in the same sheath 110. It is possible for this system to be designed such that each sheath can be placed in separate orifices (i.e., on opposite sides of the heart valve therapy). Once both loops have captured the heart valve therapy, the same steps as described above would follow.

The control member insulation that covers the outer surface of the inner control stylet and inner control member is preferred to be flexible enough to not impact the navigation of the delivery catheter through a valve orifice. It is also desirable be as lubricious as possible, such that the friction between the inner control member and the delivery catheter is minimized as the inner control member is pushed distally to deploy the basket and cutting loop in the left ventricle. For example, this insulation may include a hydrophilic coating, a silicone coating, a Teflon like coating, a polyolefin coating, a thermoform or thermoset coating, or fluoropolymers.

Returning to the basket 102, the length and diameter of the basket 102 may depend on the size of the heart valve therapy device or clip 40. For example, the basket 102 may have a length within a range of about 20 mm to about 50 mm, and a diameter within a range of about 10 mm and 20 mm. Depending on the size of the leaflet clip 40 and the angle that the basket 102 is expected to capture the clip 40, the diameter of the basket 102 can be adjusted accordingly. For example, the greater the angle of interception relative to a top plane across the opening of the basket 102, the larger the diameter of the basket 102 should be. Put another way, unless it is expected that the basket 102 is to be substantially directly underneath the clip 40, the basket 102 should expand to a diameter much greater than that of the clip 40.

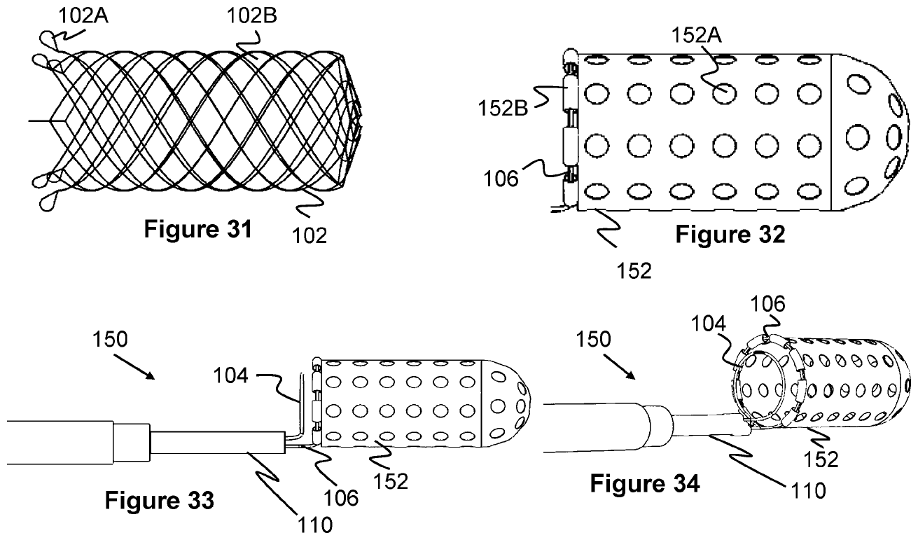

In one embodiment seen in FIG. 31, the basket 102 can be composed of a plurality of braided wires. The wires can be composed of a shape memory material and can be braided on a mandrel of a desired basket size, then heat set so that the braided shape returns to the expanded basket configuration after being compressed. The wires can be composed of a shape memory material such as Nitinol or a non-shape memory material such as stainless steel. The wires may also have an insulating coating such as ETFE, polyimide, parylene, silicone, or similar materials. The benefits of a woven basket are that its behavior/performance can be altered by changing the basket wire diameter, basket wire material, and/or weave density (i.e., basket pore size) while keeping the diameter and length of the basket fixed. The basket diameter and length design are primarily driven by the size of the intended heart valve therapy to be removed. The size, spacing, and number of woven basket eyelets could also be adjusted and optimized. In one example, the pores 102B of the basket 102 when expanded are within a range of about 100 microns to about 4 mm in diameter.

The wire size is preferably small enough to allow for it to be easily collapsed into and deployed from the delivery catheter during the procedure, but large enough to give the basket some rigidity such that it can adequately open in the presence of valve chordae or other structures. The basket pore size can vary on a woven basket, depending on the design intent. In general, the pore size should be smaller than either the length, width, or height of the heart valve therapy to avoid it embolizing through the basket after it has been cut free. Weaving a basket with very small pores could help with filtering and capturing any debris generated during the tissue cutting process.

One benefit of coating a metal basket is to ensure the electrical energy is concentrated in the cutting element and not being distributed across the entire metal structure of the basket and into the blood pool. The second benefit of coating is that it can also reduce friction and therefore can facilitate easier capture of the heart valve therapy inside the basket. If the basket is too rough or there are too many edges inside the basket, the heart valve therapy may not want to fully seat within the basket. Adding a lubricious coating or a smooth layer to the inner surface of the capture basket may enable easier capture of the heart valve therapy.

In an alternate embodiment seen in FIGS. 32, 33, and 34, a removal catheter 150 includes a basket 152 composed of a polymer such as silicone, PET, polyester, nylon, polypropylene, Kevlar, or a similar material that can fold or pleat to a radially compressed configuration. The basket 152 may be formed with a plurality of apertures that are sized to prevent passage of both the leaflet clip 40 and other biological material that may break off from the procedure (e.g., about 100 microns to about 4 mm in diameter). The basket 152 can be of similar size to the previously discussed basket 102. The top opening of the basket 152 may also include a plurality of loops or passages 152B sized to allow passage of the cinching loop 106 so that the basket 152 can be closed during a procedure.

Construction of the polymer basket 152 can be completed using a braid, mesh, weave, knit, or via injection molding. Potential basket shape and material combinations are infinite, and only a few are described here. Choosing a polymer material that has high heat resistance, low moisture absorption, and is durable enough to be collapsed into the outer sheath multiple times is important. Silicone tends to meet all of these performance requirements the best. In the event the basket is made of a silicone, it could be molded into the basket shape as a standalone component, or molded directly onto a loop structure. If creating the basket from a flat sheet of silicone, it could be cut to a designed pattern, and stitched onto a loop, into the desired shape.

The size and spacing of the pores 152A can be adjusted, depending on the material selected. In general, the pore size may be smaller than either the length, width, or height of the heart valve therapy to avoid it embolizing through the basket after it has been cut free. Using a basket with very small pores may help with filtering and capturing any debris generated during the tissue cutting process. Designing a basket with pores also allows some blood to flow through it; this helps improve the operators control of the basket by minimizing the force applied to it from pumping blood (i.e., it minimizes the ‘parachute effect’). A polymer basket could be constructed with eyelets or not; if there are eyelets as shown, it will be slidably mounted to the basket cinching loop. If there are no eyelets, it will be securely affixed to the basket cinching loop.

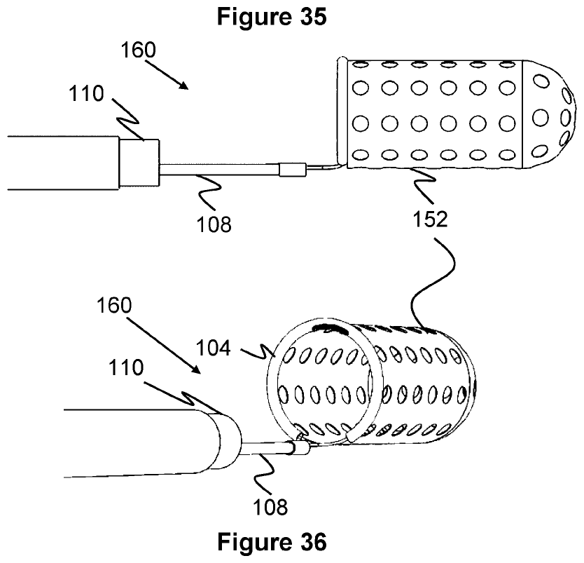

Since the polymer basket 152 does not conduct current, other embodiments are possible in which the cutting loop 104 of a removal catheter 160 also acts as a cinching loop, as seen in FIGS. 35 and 36. The basket 152 may be directly attached to the insulation portions 104A of the cutting loop 104 (or alternately may directly form the insulation portions around the uninsulated wire), leaving open the exposed, uninsulated portion 104B that performs the leaflet cutting.

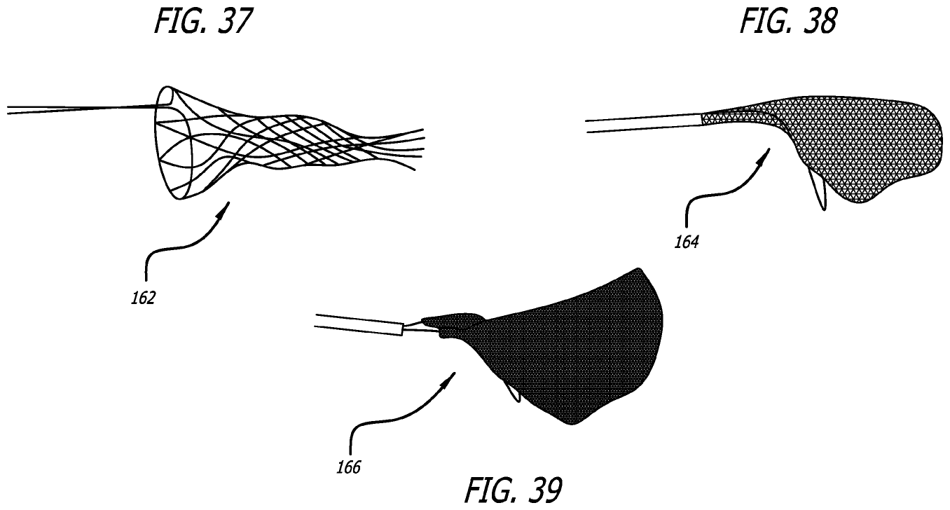

Similar “single loop” embodiments are also possible with other shapes and materials. For example, FIG. 37 illustrates a plurality of polymer or fabric filaments braided together to form a flexible basket shape and relatively large apertures (e.g., about 0.5 mm to about 4 mm). FIG. 38 illustrates a plurality of polymer or fabric fibers woven into a fabric basket 164 with relatively smaller apertures (e.g., about 0.5 mm to about 4 mm). FIG. 39 illustrates a polymer sheet that is stitched to form a basket 166. In any of these embodiments, the cutting loop 104 can be exposed so that the uninsulated portion 104B can cut through the valve leaflets after being cinched.

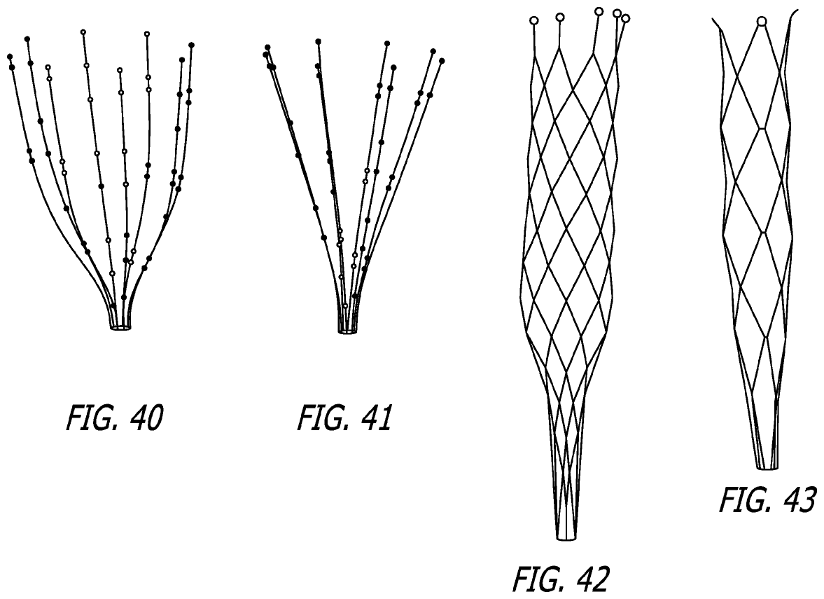

In other embodiments, the basket can be partially or fully composed of a laser cut basket. For example, FIGS. 40 and 41 illustrate a plurality of vertical, laser cut ribs with eyelet disposed along their length to allow for a plurality of wires or polymer filaments to be braided or woven through. FIGS. 42 and 43 illustrates laser cut basket shape that are entirely composed of a laser cut shape memory metal (e.g., a tube or sheet of shape memory metal).

The benefits of a laser cut basket are that its behavior/performance can be altered by changing the tube dimensions and/or cut pattern/density (i.e., basket pore size) while keeping the diameter and length of the basket fixed. The basket diameter and length design are primarily driven by the size of the intended heart valve therapy to be removed. The size, spacing, and number of laser-cut eyelets could also be adjusted and optimized. The material used preferably has shape memory properties, like Nitinol, to allow for the laser cut portion of the tube to be expanded and shaped. Using a material with shape memory is what enables the basket to collapse and open back up to the same shape, repeatedly. The wire size is preferably small enough to allow for it to be easily collapsed into and deployed from the delivery catheter during the procedure, but large enough to give the basket some rigidity such that it can adequately open in the presence of valve chordae or other structures.

Basket pore size can be varied in a laser cut design by changing the cut pattern to achieve the desired result. For example, pore sizes may vary within a range of about 100 microns to about 4 mm. In general, the pore size should be smaller than either the length, width, or height of the heart valve therapy to avoid it embolizing through the basket after it has been cut free. One unique benefit of a laser cut basket is that the pore size and spacing could vary throughout the basket length. For example, the proximal opening side of the basket could have large pores with a certain pattern density. The pore size and pattern density could get smaller and denser towards the distal end of the basket.

Any of the basket embodiments described in this specification can further include an outer covering to help collect any debris or embolic material freed during the procedure. Such an outer covering may include a solid or perforated polymer sheet, a woven fabric, a tubular shape formed from relatively small, finely braided metal wires, or similar materials. In one specific embodiment, the interior of the basket can have a nonconductive liner, film, or coating (e.g., silicone) on its inner surface to help prevent conduction with the cutting element 104.

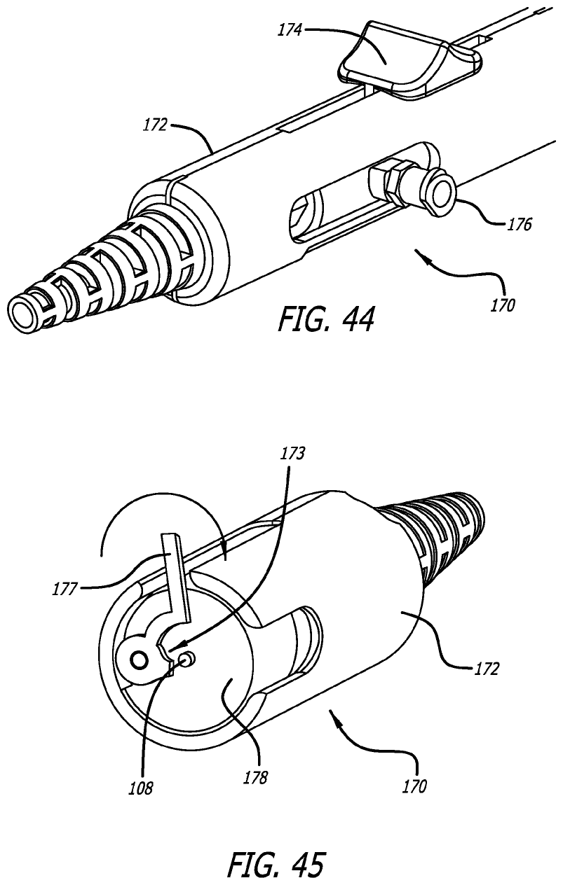

In one embodiment, the removal catheter 100 can include a proximal handle portion 170, as seen in FIGS. 44 and 45. The handle 170 includes an outer housing 172 and a sliding member 174 that is configured to slide within a longitudinal slot within the housing 172. The housing 172 can be connected to the outer tubular sheath 110 while the sliding member 174 is connected to the inner control member 108, thereby allowing the user to adjust the position of the sliding member 174 with their thumb to make a corresponding longitudinal move of the inner control member 108, basket 102, and cutting loop 106.

Optionally, the handle 170 may also include a fluid connection port 176 (e.g., a luer port) that is in communication with an interior of the interior passage of the outer tubular sheath 110 so that an electrically neutral solution (e.g., a dextrose solution) can be delivered to the area near the cutting loop, amplifying the tissue cutting effects and minimizing energy loss around the area to the blood pool. The amount and timing of this fluid can be determined by a physician (e.g., via a syringe) or via an electrically actuated pump mechanism based on a position of the cutting loop 106 (i.e., when the cutting loop is outside of the outer tubular sheath and in good contact with desired tissue 110).

As seen in FIG. 45, the handle 170 may also include a locking mechanism 173 near a distal end of the housing 172 which locks the inner control member 108 in place relative to the outer tubular sheath 110. For example, the locking mechanism 173 can include a handle 177 that is configured to rotate a cam member 178 that surrounds a proximal end of the inner control member 108. When the handle 177 rotates the member 178, the cam member 178 creates an interference fit with the inside of the housing 172, locking the control member 108 in its longitudinal position. When the handle 177 rotates the cam member 178 in the opposite direction, it releases the interference fit between the cam member 178 and the housing 172 to release the inner control member 108 so that it can longitudinally slide within the handle 170.

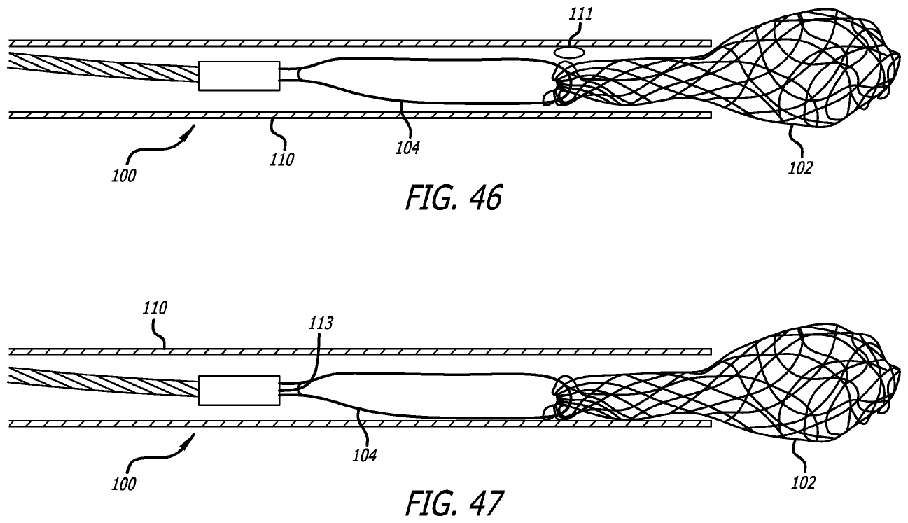

FIG. 46 illustrates an embodiment of the removal catheter 100 with a current over-flow hole 111 in the outer tubular sheath 110. This embodiment is most beneficial for an embodiment with a single cinch and cutting loop and either a polymer or minimally conductive basket 102. As the cutting element 104 and affixed basket 102 are retracted inside the outer tubular sheath 110 to begin cutting the leaflet tissue, the distal end of the outer tubular sheath 110 can become closed off from the blood pool. If this happens after the tissue has been cut, the current being delivered to the cutting element 104 is no longer being transferred to tissue or blood, and instead transfers the heat and/or electricity through the basket 102 and can damage it. The current overflow hole 111 in the outer tubular sheath 110 can ensure the cutting element 104 is always in communication with the blood pool, even after the cut has been completed. In this way, the current will choose to flow through the blood to the opposite RF electrode attached elsewhere on the patient, as opposed to the basket.

Alternately, a wire 113 can be attached to the inner control member 108 to ensure the current pathway always involves the blood pool, even after the cut has been completed, as seen in FIG. 47. The wire 113 is preferably designed to be long enough to always protrude from the distal end of the outer tubular sheath 110, even with the basket 102 fully collapsed inside the outer tubular sheath 110. It would also preferably have a very small region of exposed metal at the very distal tip, and the rest would be insulated. In this way, when the cut is completed the current will choose to flow through the lower resistance wire and to the blood as opposed through the higher resistance basket 102 (e.g., silicone).

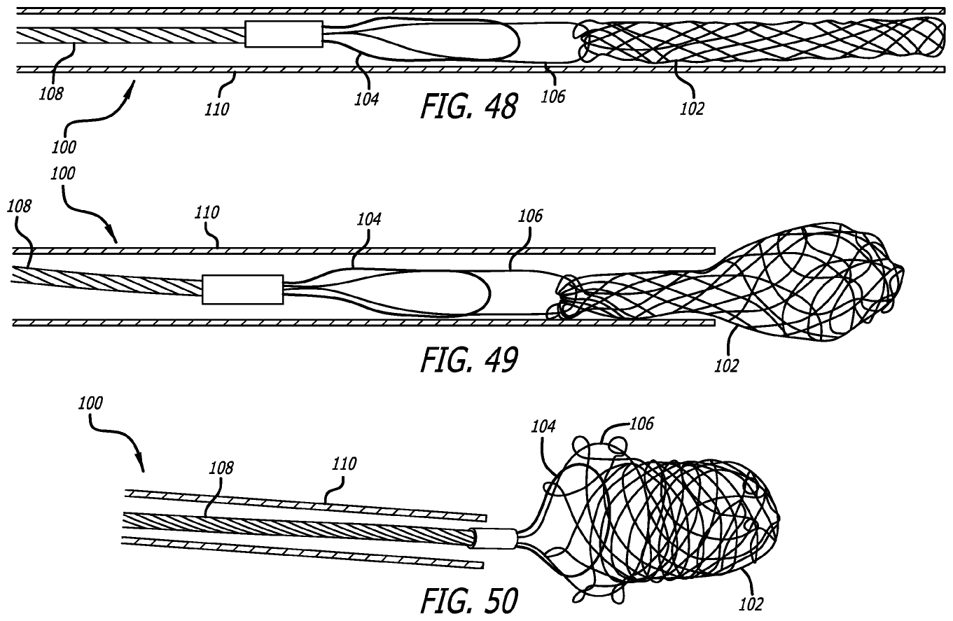

FIGS. 48-53 illustrate side views of the removal catheter 100 deploying and cinching its basket 102. In FIG. 48, the basket 102, cinching loop 106, and cutting loop 104 are all located within the outer tubular sheath 110. As can be seen in this Figure, these components are radially compressed to a relatively smaller diameter to allow passage through the vessels of a patient (keeping them compressed in outer tubular sheath enables passage through smaller orifices and between multiple clips as well).

In FIG. 49, the inner control member 108 is distally advanced (e.g., via sliding member 174) so that the basket 102 begins to exit the outer tubular sheath 110 and radially expand. This distal movement continues until both the basket 102 and the cutting loop 104 have deployed and fully expanded outside the sheath 110, as seen in FIG. 50.

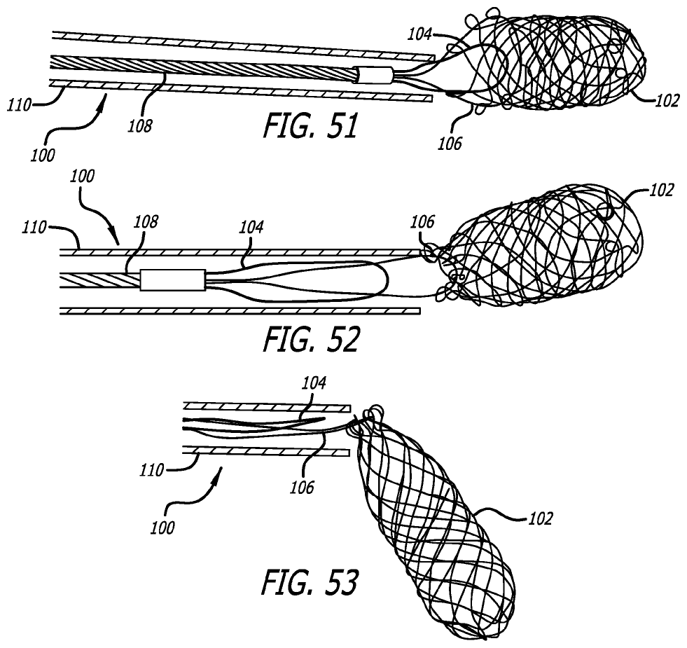

FIGS. 51 and 52 illustrate the inner control wire 108 being retracted, causing both the cinching loop 106 and the cutting wire 104 to retract and radially close in diameter. Typically, RF energy will be activated during this time so that as the cutting wire 104 closes, it cuts the tissue of the leaflets. As the cutting loop 104 is fully pulled inside the outer tubular sheath 110, the RF current is deactivated. This can be achieved in a plurality of different ways. For example, the previously described sliding member 174 of the handle 170 may include a position switch that turns the RF energy on/off at a predetermined longitudinal position. Alternately, a manual on/off switch can be included on the handle 170 or RF power supply.

To assist in determining when to manually turn off the RF energy, a radiopaque marker can be placed at the distal end of the outer tubular sheath 110. As the physician performs the tissue bridge cut, they will have their eyes on the fluoroscopy screen. Since tissue is typically not visible on fluoroscopy, providing the operator with a visual indicator on the catheter 100 indicating that the tissue bridge has been cut may be useful. The inner control member 108 and cutting loop 104 are retracted into the sheath 110 during the cutting process and the radiopaque marker is located such that when the operator sees on fluoroscopy the entire cutting loop 104 on the proximal side of the radiopaque marker, the tissue bridge has been cut. Not only is this a useful visual indicator for the operator, but it also makes the procedure safer. Once the cutting loop 104 has passed the radiopaque marker, the RF cutting energy can be terminated immediately by the operator to prevent any unintended heating by applying power longer than necessary.

Finally, the opening of the basket 102 is nearly completely cinched closed and the positioned of the inner control member 108 may optionally be locked in place (e.g., with locking mechanism 173 on the handle 170). The basket 102 may be maintained outside of the outer tubular sheath 110 and pulled into a larger guide catheter used during the procedure.

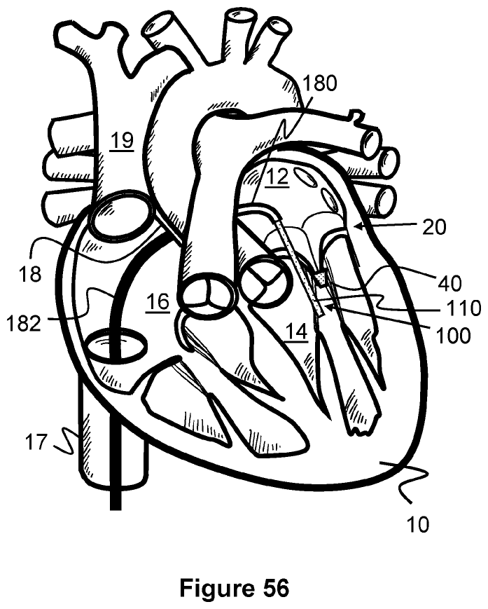

The present invention includes different methods or approaches of removing a heart valve therapy such as a valve clip 40. For example, FIGS. 56-61 illustrate a removal procedure in which the atrial septum 18 is crossed to access the mitral valve 20. While example access methods and procedures are described, it should be understood that variations are possible based on known catheter access techniques. Additionally, these access techniques can be used with any of the embodiments of this specification.

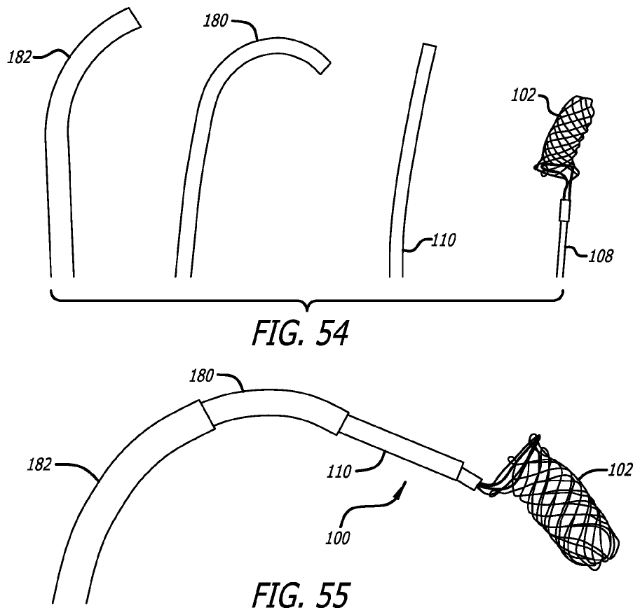

The mitral valve access procedure of FIGS. 56-61 may, in one embodiment, include an inner control member 108, an outer tubular sheath 110, an inner steerable catheter 180, and an outer transseptal guide catheter 182, which can be seen separately in FIG. 54 and together in FIG. 55 and are discussed further below. The three nested but independent curving and axially articulating catheters make it possible to position the removal device anywhere in the heart regardless of size or procedural positioning. However, other tools, sheaths, catheters, and similar devices may alternately be used to directed the removal catheter 100 as described below.

Turning first to FIG. 56, the left atrium 12 can be accessed by advancing a transseptal guidewire or needle to the atrial septum 18 (e.g., via the inferior vena cava 17 or the superior vena cava 19), using the guidewire to cross the atrial septum 18, and finally moving the guidewire into the left atrium. Next, a relatively larger diameter outer transseptal guide catheter 182 can be advanced over the guidewire and through the atrial septum 18 so the its distal end is located in the left atrium 12. Alternately, the transseptal guide catheter 182 can be advanced through the atrial septum 18 without the use of any transseptal guidewire. The outer transseptal guide catheter 182 may optionally have a predetermined curve or bend that may help angle it from the inferior vena cava 17 towards the atrial septum 18.

The guidewire can be removed and the inner steerable guide catheter 180 can then be advanced through the outer transseptal guide catheter 182 so that its distal end is located within the left atrium 12. The distal end of the inner steerable guide catheter 180 can be “steered” or deflected so that its distal opening is directed toward a desired location of the mitral valve 20. Since the guide catheter is independent of the outer transseptal guide catheter 182, the physician has the ability to direct the inner steerable guide catheter 180 to any location along the mitral valve 20, such that it can be rotated, advanced/retracted, or have the degrees of deflection altered while keeping the outer transseptal guide catheter 182 in the same location.

In the example of a mitral valve 20 having a leaflet clip 40, the inner steerable guide catheter 180 is preferably pointed towards either of the two valve openings on each side of the center clip 40 (see top view of FIG. 6). Once pointed at the desired target location, the removal catheter 100 is advanced through the inner steerable guide catheter 180 and out its distal end, into the left atrium 12, through one of the side openings of the mitral valve 20, and into the left ventricle 14, as seen in FIG. 56.

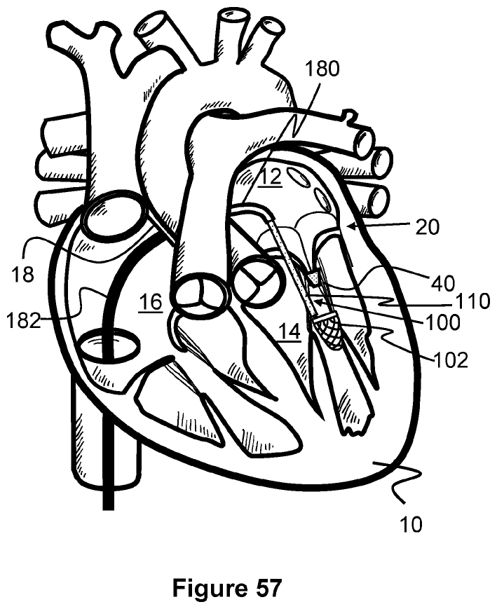

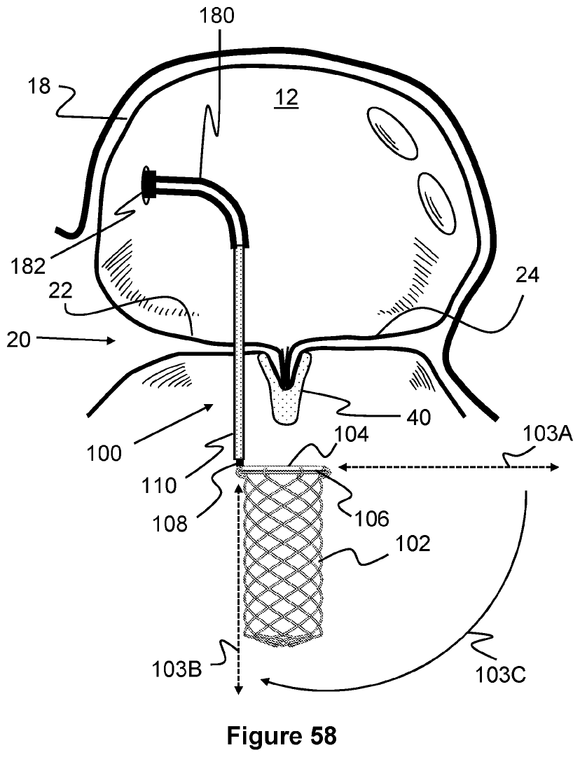

As seen in FIGS. 57 and 58, the inner control member 108 is distally advanced through the outer tubular sheath 110 of the removal catheter 100, causing the capture basket 102, cinching loop 106, and the cutting loop 104 to be advanced out of the outer tubular sheath 110. Preferably the capture basket 102, cinching loop 106, and the cutting loop 104 are connected to the inner control member 108 so that they expand to an orientation in which the opening of the basket 102 and the opening of the cutting loop 104 are directed or point towards the leaflet clip 40. For example, the plane 103A of the opening of the basket 102 and the opening of the cutting loop 104 may be an angle 103C between 45 degrees and 135 degrees relative to an axis 103B of the inner control member 108 (e.g., 90 degrees). The inner control member 108 can be rotated relative to the outer tubular sheath 110 (or alternately the entire removal catheter 100 can be rotated) to cause the capture basket 102, cinching loop 106, and the cutting loop 104 to also rotate within the left ventricle 14. In this manner, the physician can align or orient the basket 102 to a desired location directly beneath the leaflet clip 40.

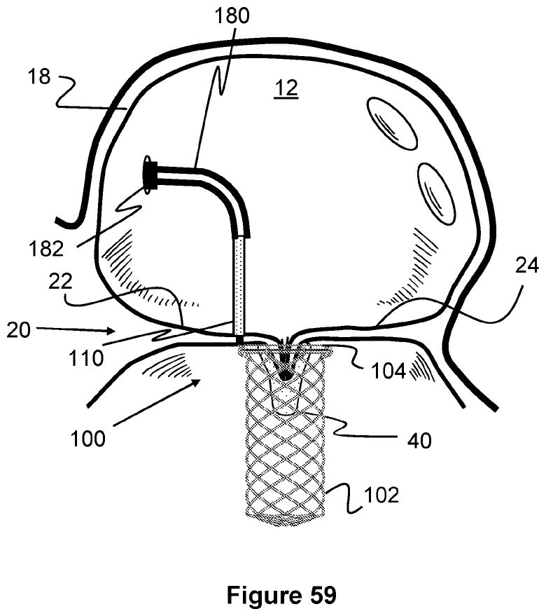

Once the capture basket 102, cinching loop 106, and the cutting loop 104 are deployed, the inner control member 108 (or alternately the outer tubular sheath 110) can be proximally withdrawn so that the leaflet clip 40 is positioned inside of the basket 102, as seen in FIG. 59. Preferably, both the cutting loop 104 and the cinching loop 106 are positioned above the leaflet clip 40; that is between the leaflet clip 40 and the bottom adjacent surfaces of the leaflets 22 and 24.

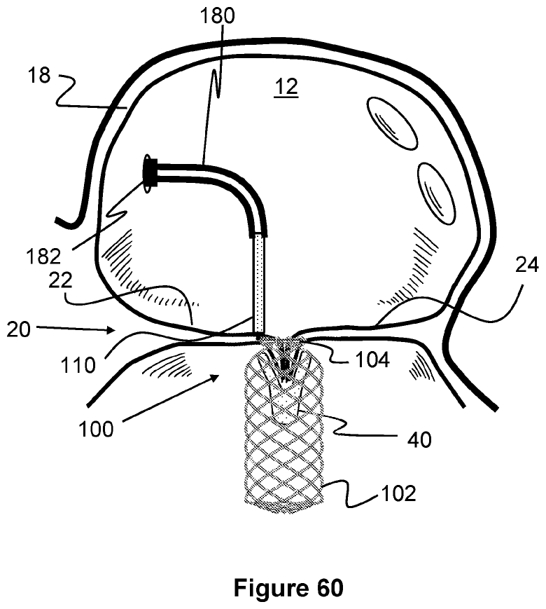

Turning to FIG. 60, the inner control member 108 is proximally retracted so as to partially retract the cinching loop 106 and the cutting loop 104. This causes the top opening of the basket 102 to close in diameter above the leaflet clip 40 and also causes the cutting loop 104 to reduce diameter and engage between an atrial side portion of the leaflets 22, 24 and the leaflet clip 40.

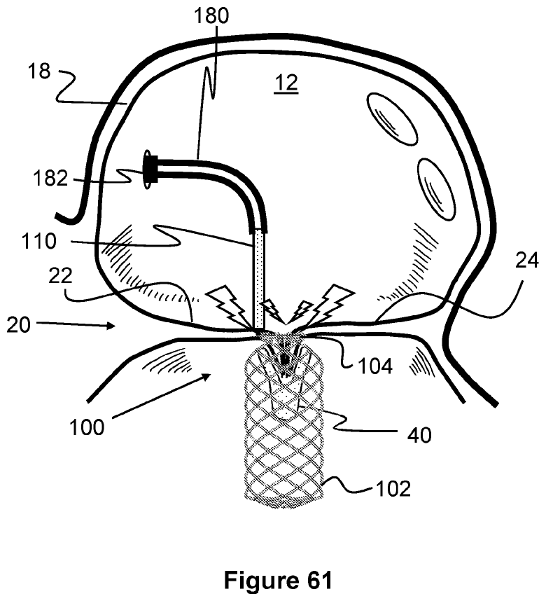

As seen in FIG. 61, as the cutting loop 104 is proximally withdrawn and decreased in diameter, RF energy is applied to the cutting loop 104. The uninsulated portion 104B presses against portions of the leaflets 22 and 24 nearest to the leaflet clip 40, thereby cutting this tissue and freeing the leaflet clip 40 from the mitral valve 20. The RF energy is turned off to the cutting loop 104. Preferably, the outer transseptal guide catheter 182 has a large enough diameter to allow the basket 102 containing the leaflet clip 40 within it. However, the removal catheter 100, inner steerable catheter 180, and outer transseptal guide catheter 182 can all be withdrawn from the patient as a single unit, if necessary.

It is further contemplated that, after removal of the leaflet clip 40, an artificial valve may be installed at the location of the mitral valve 20. If a guidewire is used during the removal procedure, it can also be used to advance and orient a valve delivery catheter to delivery and implant the artificial valve. One example of such an artificial valve replacement can be found in U.S. Pat. No. 8,579,964, entitled Transcatheter Mitral Valve Prosthesis, the content of which is hereby incorporated by reference.

It is further contemplated that, after removal of the leaflet clip a blood flow management apparatus such as a spacer, catheter, balloon, or other device is in and could be expanded in the location of the valve to manage the flow across the valve until such time as additional therapy could be delivered such as a replacement valve.

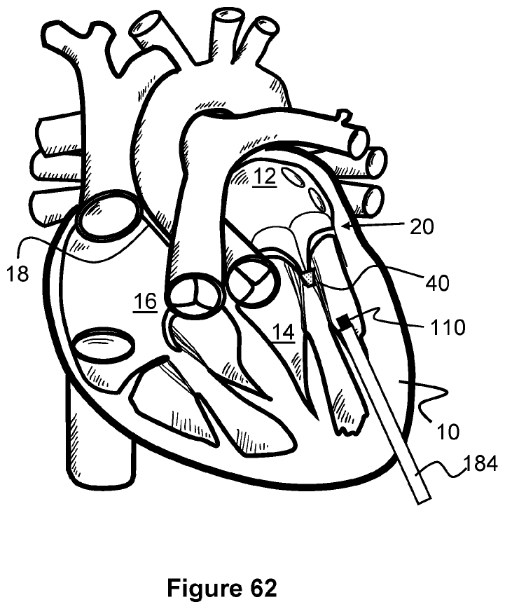

FIGS. 62-65 illustrate another method of removing a leaflet positioning device such as a leaflet clip 40 via a transapical approach. First, an incision is made in the sternum (e.g., between the manubrium and the sternum) and a transapical sheath 184 is advanced through the incision, through the apex of the heart 10, and into the left ventricle 14, as seen in FIG. 62. The removal catheter 100 is then advanced through the transapical sheath 184 so that a distal end of the outer tubular sheath 110 extends out into the left ventricle 14.

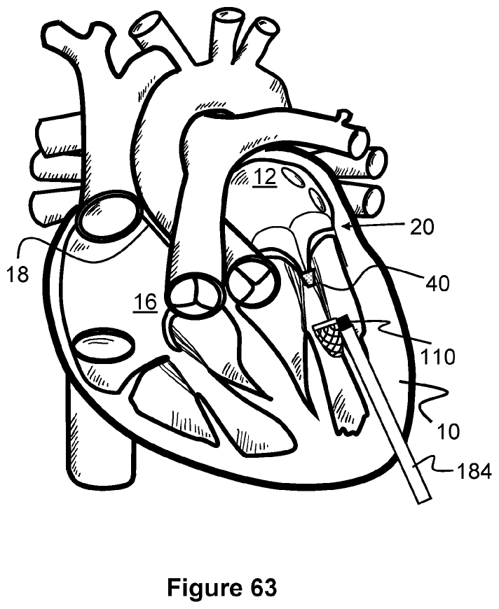

Turning to FIG. 63, the inner control member 108 is distally advanced within the outer tubular sheath 110 so as to release and expand the basket 102 and cutting loop 104 into the left ventricle 14. The cinching loop 106 and the cutting loop 104 preferably have a predetermined bend (e.g., a heat set bend/curve) that orients the top opening of the basket 102 and the opening of the cutting loops 104 towards the leaflet clip 40. For example, the plane of the top opening of the basket 102 and the opening of the cutting loops 104 can be within a range of 135 degrees to 225 degrees (e.g., about 180 degrees) relative to an axis of the inner control member 108. The inner control member 108 can be further rotated (or the entire removal catheter 100 can be rotated) by the physician so as to best align the cutting loop 104 and basket 102 with the leaflet clip 40.

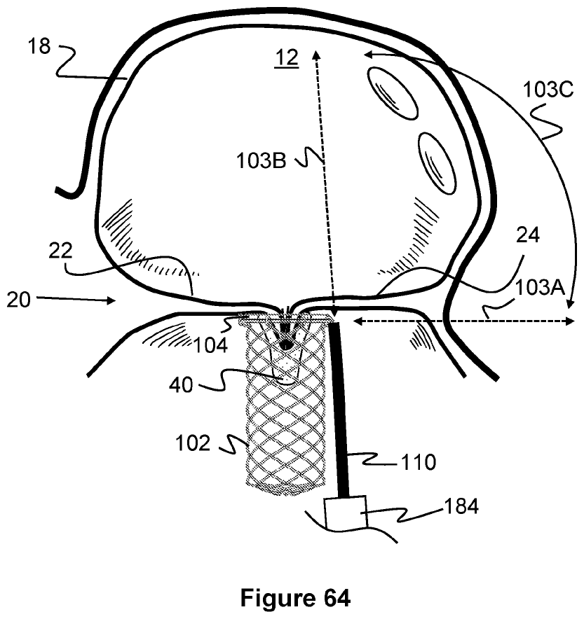

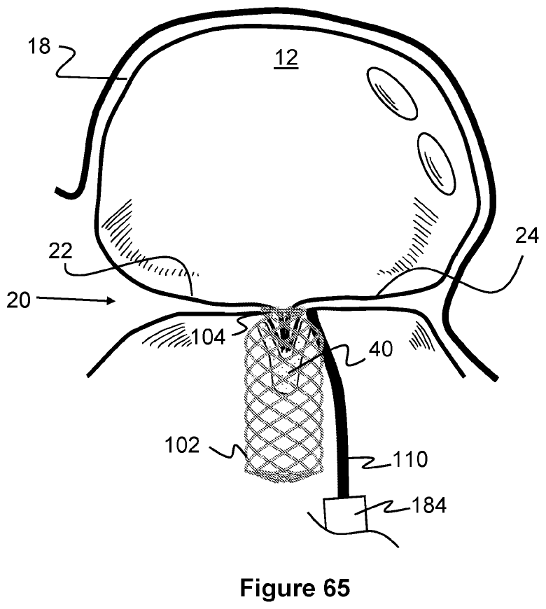

As seen in FIG. 64, the outer tubular sheath 110 is further advanced out of the transapical sheath 184 so that the leaflet clip 40 is positioned completely within the basket 102. As seen in FIG. 65, the inner control member 108 is proximally retracted to cause the cinching loop 106 and the cutting loop 104 to decrease in diameter. As the loops 104 and 106 decrease in diameter, the top opening of the basket 102 decreases, trapping the leaflet clip 40 within it. Additionally, as the cutting loop 104 decreases, RF energy is activated and delivered to the loop 104, allowing the uninsulated portion 104B to cut through the leaflet tissue immediately above the leaflet clip 40.

Preferably the capture basket 102, cinching loop 106, and the cutting loop 104 are connected to the inner control member 108 so that they expand to an orientation in which the opening of the basket 102 and the opening of the cutting loop 104 are directed or point towards the leaflet clip 40. For example, the plane 103A of the opening of the basket 102 and the opening of the cutting loop 104 may be an angle 103C between 25 degrees and 135 degrees relative to an axis 103B of the inner control member 108 (e.g., 90 degrees).

If the transapical sheath 184 has a large enough diameter, the outer tubular sheath 110 can be proximally retracted and the basket 102 containing the leaflet clip 40 is withdrawn into the passage of the transapical sheath 184 for removal. If the basket 102 and leaflet clip 40 are too large for the transapical sheath 184, both the sheath 184 and the removal catheter 100 can be pulled out together simultaneously.

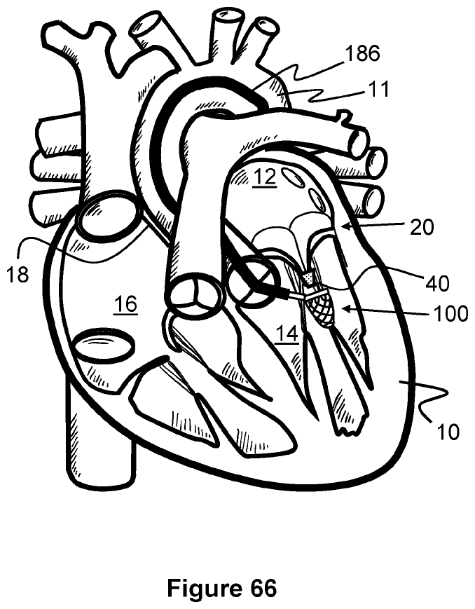

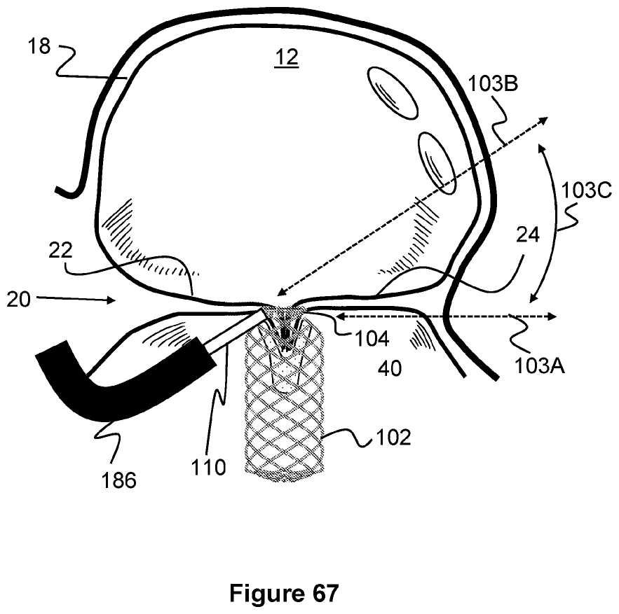

FIGS. 66 and 67 illustrate another method of removing a heart valve therapy such as a leaflet clip 40 via a transaortic approach. Referring to FIG. 66, an aortic guide catheter 186 is first positioned into the aorta 11 and advanced into the left ventricle 14. The aortic guide catheter 186 may have a fixed curve/shape that helps the physician direct the distal end of the catheter 186 beneath the leaflet clip 40. Alternately or additionally, the aortic guide catheter 186 may include steerable mechanisms to allow deflection in different directions.

Next, the removal catheter 100 is advanced through the aortic guide catheter 186 so that a distal end of the outer tubular sheath 110 extends from the distal end of the catheter 186 and into the left ventricle 14. The inner control member 108 is further distally advanced relative to the outer tubular sheath 110 so that the basket 102 and cutting loop 104 are deployed, expanded, and positioned in the left ventricle 14. The opening of the basket 102 and the opening of the cutting loop 104 are both or oriented so that they face the leaflet clip 40. For example, the face of the opening of the basket 102 and the opening of the cutting loop 104 may be within a range of about 300 degrees and 45 degrees relative to an axis of the inner control member 108 (e.g., about 320 degrees).

Referring to FIG. 67, the aortic guide catheter 186 is either moved or deflected (in the case of a steerable catheter) so that the cutting loop 104 and basket 102 are positioned over the leaflet clip 40. The inner control member 108 is proximally retracted inside 110, causing the cinching loop 106 and the cutting loop 104 to decrease in diameter, closing the top opening of the basket 102. As the cutting loop 104 decreases in diameter, RF energy is delivered to the loop 104, allowing the uninsulated portion 104 to cut areas of the leaflet tissue adjacent to the leaflet clip 40 and thereby freeing the leaflet clip 40 from the mitral valve 20. The basket 102 and leaflet clip 40 can either be retracted through the aortic guide catheter 186 or all of the catheters can be removed together as a single unit simultaneously.

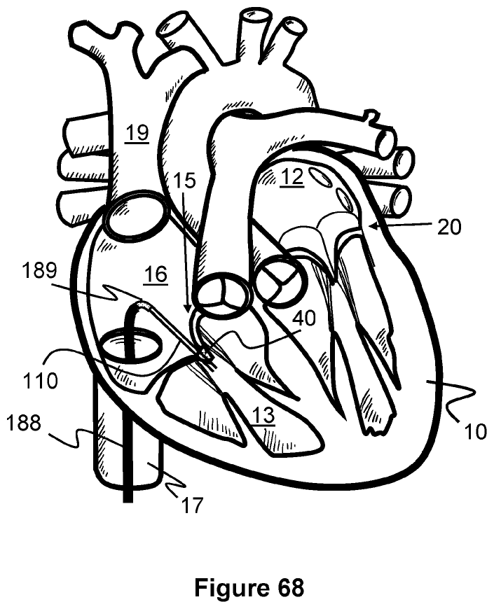

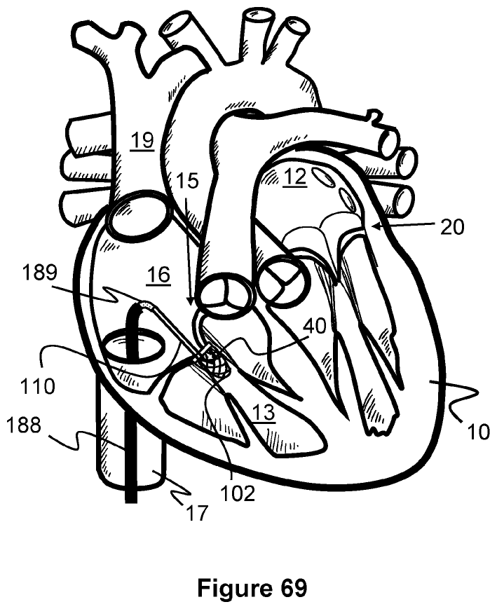

The present invention also contemplates using the removal catheter 100 (or any of the variations described in this specification) on the tricuspid valve 15, as seen in FIGS. 68 and 69. Referring first to FIG. 68, an outer tricuspid guide catheter 188 is first delivered to the right atrium 16 by either an approach through the inferior vena cava 17 or the superior vena cava 19. The tricuspid guide catheter 188 may include a fixed curve at its distal end to help its distal opening towards the tricuspid valve 15 or can include steering mechanisms to perform the same. An inner intermediate catheter 189 can then be advanced through the outer tricuspid guide catheter 188 to provide a better angle towards the tricuspid valve 15. For example, the inner intermediate catheter 189 may have a fixed curve towards the tricuspid valve 15 or may include steerable catheter mechanisms to allow the physician to deflect the distal end of the catheter 189 towards the tricuspid valve 15.

Next, the removal catheter 100 is advanced through the inner intermediate catheter 189 so that it passes out of the distal end of the inner intermediate catheter 189, into the right atrium 16, through the tricuspid valve 15, and into the right ventricle 13. Since the leaflet clip 40 is typically positioned in the middle of the valve 15 (e.g., similar to the top view of the mitral valve in FIG. 6), creating to side valve openings, the removal catheter 100 is preferably positioned on either side of the leaflet clip 40.

The inner control member 108 is further distally advanced relative to the outer tubular sheath 110 so that the basket 102 and cutting loop 104 are deployed, expanded, and positioned in the right ventricle 13. The opening of the basket 102 and the opening of the cutting loop 104 are both or oriented so that they face the leaflet clip 40. For example, a plane 103A of the face of the opening of the basket 102 and the opening of the cutting loop 104 may be an angle 103C within a range of about 0 degrees and 90 degrees relative to an axis 103B of the inner control member 108 (e.g., about 45 degrees). The removal catheter 100 is proximally retracted relative to the inner intermediate catheter 189, so that the cutting loop 104 and basket 102 are positioned over and beyond the leaflet clip 40.

The inner control member 108 is proximally retracted, causing the cinching loop 106 and the cutting loop 104 to decrease in diameter, closing the top opening of the basket 102. As the cutting loop 104 decreases in diameter, RF energy is delivered to the loop 104, allowing the uninsulated portion 104 to cut areas of the leaflet tissue adjacent to the leaflet clip 40 and thereby freeing the leaflet clip 40 from the tricuspid valve 15. The basket 102 and leaflet clip 40 can either be retracted through the inner intermediate catheter 189 or all of the catheters can be removed together as a single unit simultaneously.

It should be understood that any of the embodiments of the present specification can be used according to the access and delivery methods described in this application. Additionally, further methods can be used with these access and delivery methods, such as delivery and implantation of an artificial valve (either mitral or tricuspid valve).

While the previously described removal catheter embodiments have included a basket or similar device to capture the heart valve therapy, such as a leaflet clip 40, different capture approaches and devices are also contemplated.

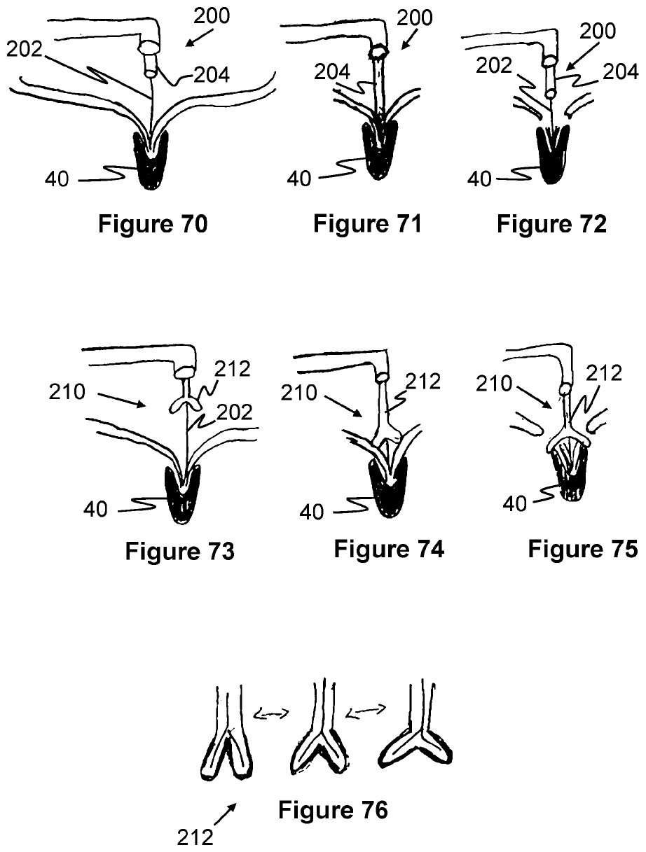

FIGS. 70-72 illustrates a removal catheter 200 for removing a heart valve therapy leaflet clip 40. The removal catheter 200 includes an elongated piercing member 202 that pierces into the device, and an outer cutting catheter 204 that is disposed over the piercing member 202. The elongated piercing member 202 can be a wire, catheter or similar elongated device having a distal end that is sharpened, helically shaped, an expandable barb, or rotational elements, such that the elongated piercing member 202 can be pressed into a top of the leaflet clip 40 (and optionally rotated or expanded) to initially engage or capture the leaflet clip 40, as seen in FIG. 70.

As seen in FIG. 71, the outer cutting catheter 204 is distally advanced over the elongated piercing member 202 until its distal end contacts the top surface of the valve leaflets, as seen in FIG. 71. The outer cutting catheter 204 can be configured to cut the leaflet tissue with a variety of different mechanisms, such as mechanical (e.g., rotation or forward pressure) and/or electrosurgical cutting device (i.e., electrical or cryo). As seen the FIG. 72, once freed from the leaflet tissue, the leaflet clip 40 can be removed by the elongated piercing member 202.

FIGS. 73-75 illustrate a removal catheter 210 that is similar to the previously described catheter 200, except that the cutting catheter 212 also includes a grasping mechanism having to two articulating jaw members connected via a joint (FIG. 76). After the elongated piercing member 202 has engaged the leaflet clip 40 (FIG. 73), the outer cutting member 212 is distally advanced over the elongated piercing member 202 until it contacts the top surface of the leaflets (FIG. 74). The articulating jaw members preferably include tissue cutting mechanisms on their ends, such as blades or electrical/cryo electrosurgical cutting device mechanisms, allowing the leaflet tissue around the leaflet clip 40 to be cut. Finally, in FIG. 75, the jaw members of the cutting catheter 212 are brought towards each other to engage and grasp the tissue clip 40.

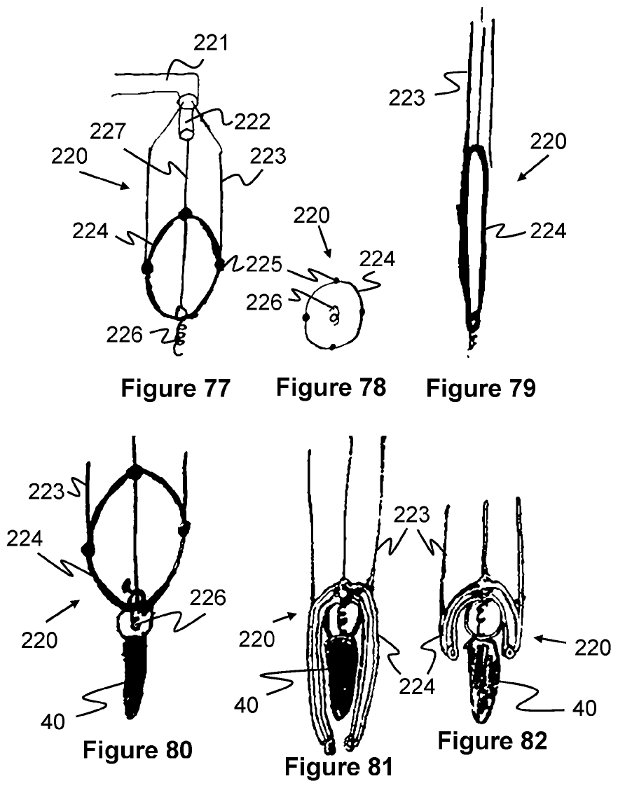

FIGS. 77-82 illustrates another embodiment of a removal catheter 220 that embeds in the previously placed leaflet clip 40, followed by passage of a loop-based tool 224 that encapsulates, cuts, and removes the clip 40. In FIG. 77, the loop-based removal catheter 220 includes an anchoring mechanism 226 connected to a central push rod 227, side push rods 223, and pushability elements 225.

FIG. 78 illustrates an end face view of the loop-based removal catheter 220, which may be circular, oval, multi-segmented, or a combination of these shapes and elements. The side rods 223 push on pushability elements 225, while the central push rod 227 applies force to anchoring mechanism 226. In FIG. 79, the entire loop-based removal catheter 220 is folded for placement inside delivery catheter sheath 221. In FIG. 80, the anchoring mechanism 226 is advanced into the heart valve therapy hardware (i.e., leaflet clip 40) using the central push rod 227 and pushability element 125. In FIGS. 78 and 79, the side push rods 223 then are used push on the pushability element 225 to wrap the loop-based removal catheter 220 completely or partially around the leaflet clip 40. Cutting is performed mechanically or electrically followed by removal of the targeted tissue.

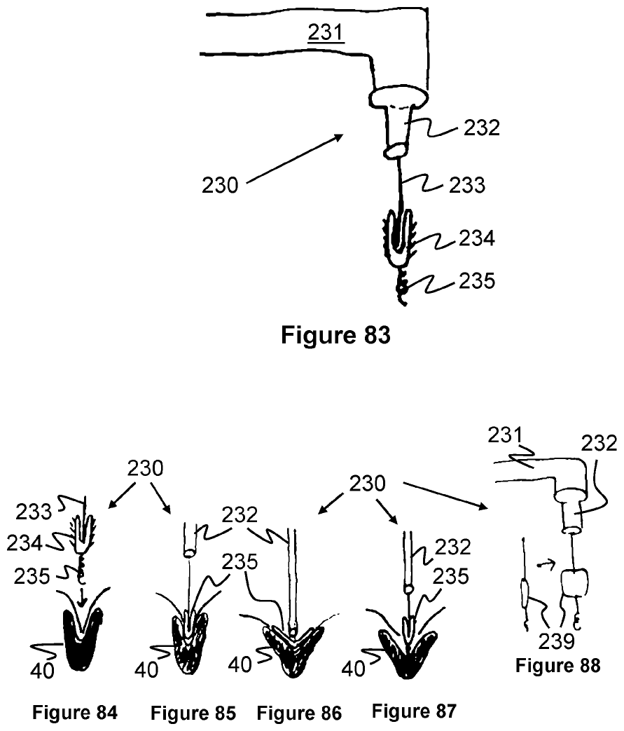

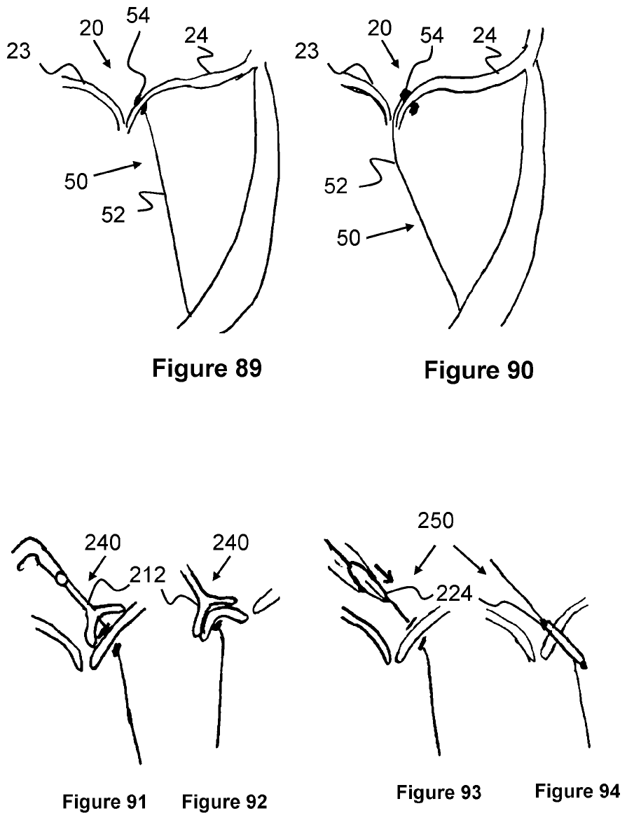

FIGS. 83-88 illustrate a removal catheter 230 that embeds in the previously placed heart valve therapy (e.g., leaflet clip 40), followed by passage of a tool that expands the leaflet clip 40, followed by removal of the hardware. In FIG. 83, the steerable guide catheter 231 is used to position the removal catheter 232, which contains the expanding tool 234 for expanding the leaflet clip 40. The expanding tool 234 may have barbs, anchors, or embedding mechanisms to remove or grasp native or foreign, leaflet or tissue material from the tissue clip 40. In FIG. 84, an anchor 235 is advanced and implanted. In FIG. 85, the expanding tool 234 is advanced inside the leaflet clip 40. In FIG. 86, the expanding tool 235 is mechanically expanded to expand the leaflet clip 40. The expansion may be aided by electrification, heating, hydraulic means, rotation, internal or external ultrasound or energy. The tissue is cut from the leaflet clip 40. In FIG. 89, the expansion tool 235 is closed and then removed, free from the leaflets. FIG. 88 shows a similar approach with a balloon expandable element 129.

FIGS. 89 and 90 are cross-sectional views of the mitral valve 20 that has been treated with a heart valve therapy comprising one or more chordal structures 50. The chordal structures 50 typically include a chord or strand 52 that is connected to a leaflet via anchors 54 and to the left ventricle. The cord 52 may be anchored on the ventricular side of the leaflet 24 (FIG. 89) or the atrial side of the leaflet 24 (FIG. 90). As further described in the embodiments below, similar device can be used to remove the chordal structures 50 as were used to remove a leaflet clip 40.

FIGS. 91-92 illustrate a removal tool 240 for cutting and capturing previously placed heart valve therapy involving cord or chordal structures implanted into the leaflets. In FIG. 91, the procedure is performed with a previously described cutting catheter 212 with capabilities of opening, closing, electrification, and removal of the hardware, similar to the above description for other heart valve therapy in FIGS. 73-76.

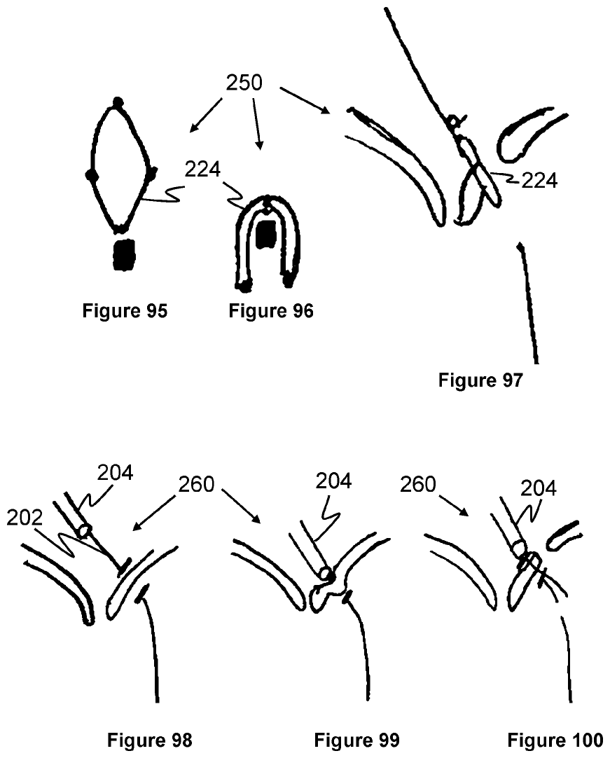

In FIG. 93-97, the procedure is performed with passage of a loop-based tool 250 that encapsulates, cuts, and removes the hardware, similar to the above description of FIGS. 77-82.

In FIGS. 98-100, the procedure is performed with a cutting catheter 260, similar to the above description for other heart valve therapy seen in FIGS. 70-72.

Additionally, a flow limiter can be used to help limit flow during any of the procedures described in this specification. For example FIG. 109 illustrates the embodiment of the removal catheter 100 of FIG. 60 with an additional flow limiting device 341. This flow limiting device 341 can be positioned within the region of valve 20 (e.g., through the valve 20) before, during, or after the removal of the clip 40 and is maintained in the valve region to manage the blood flow of the patient by limiting the blood flow. The flow limiting device 341 can be any flow limiter known to those familiar with the art, such as but not limited to, a balloon, a stent with covering, or a catheter. Any or all of these examples being configured to or have the ability to expand to occupy the clinically appropriate space to manage the blood flow by itself or in combination with the valve structure. The flow limiting device 341 can be introduced independently as shown here via delivery catheters 342 and 343. Additionally, the flow limiting device 341 can be integral into the delivery mechanism, such as the inner steerable catheter 180. Alternatively, the flow limiting device 341 can be the delivery system of another therapy such as but not limited to a heart valve.

It can be helpful when performing the cutting procedures of this specification to maintain good mechanical contact or force between the leaflet tissue and the cutting element (e.g., cutting wire 104), particularly when using RF energy as the mechanism for performing the cutting. This contact or force can be important for ensuring that the electrode of the cutting element/loop is not exposed to a large amount of blood.

When using a constant power generator, such as many commercially available RF energy generators, impedance seen by the generator will typically be low (e.g., 0-300 Ohms) when a cutting element is mostly exposed to the patient's blood. This low impedance may result in an insufficient voltage to cut through the tissue. In such circumstances, some RF energy generators will increase the current to maintain a constant power level.

In contrast, when the cutting element is pressed firmly against the patient's tissue and exposed to little, if any, of the patient's blood, the impedance seen by the generator will be relatively higher (e.g., greater than 300 Ohms) or more resistive to the current. This greater resistance may cause the generator to increase the voltage output to try to maintain constant power. Once the voltage reaches a certain level, the tissue cutting process begins and will continue so long as the cutting element is continuously in contact with target tissue.

Capitalizing on this phenomenon is what enables electrosurgery to be performed within the heart and patient's blood pool. Hence, it can be helpful in the context of the present invention to include a mechanism that creates and/or helps maintain pressure or force between the cutting element and the target tissue.

Generally, when considering the removal of an implanted valve clip 40, it can be helpful to consider several factors to ensure adequate mechanical force between the cutting element and the target leaflet tissue. Specifically, 1) the length of the leaflet tissue that is inserted into the arms of the valve clip 40, 2) the chords extending from and around the valve clip 40, and 3) the use of multiple valve clips 40 and their spacing and position angles.

Further, there are potentially at least four different valve clip removal scenarios that may be encountered. Specifically, 1) a cutting loop may be cinched on the atrial side of the valve clip 40 without the need for any further “counter force” against the clip 40 (as described in earlier embodiments/methods of this specification), 2) a counter force on the valve clip 40 may be helpful or needed to stretch the tissues such that the cutting loop can be cinched on the atrial side of the valve clip 40, 3) a counter force may be helpful or needed and the cutting loop must first cut through some leaflet side tissues before it can be cinched on the atrial side of the valve clip 40, and 4) a cutting snare can be used to substantially release the engaged tissue from the clip 40 and then can be used to facilitate engagement of cutting loop and basket.

In the first scenario, it may be possible to place the cutting loop and basket over the valve clip 40 and cinch the cutting loop on the tissue bridge such that the cutting loop is positioned on the atrial side of the valve clip 40 without the use of any further tools, as has been previously described in this specification. This technique was described earlier in this specification. Cinching the cutting loop in this way ensures the cutting element is firmly pressed against tissue and will move through the entire tissue bridge as it is pulled inside the delivery catheter.

In the second scenario, some method of pushing or pulling the valve clip 40 is provided as the cutting loop is retracted or cinched. This counter force helps stretch the tissue into/towards the left ventricle 14 and away from the direction that the cutting loop is being pulled and therefore allows the cutting loop to be positioned and cinched on the atrial side of the valve clip 40. Applying this counter force can be helpful and sometimes even necessary if a relatively large length of leaflet tissue has been inserted into the arms of the clip 40, multiple clips 40 have been implanted (especially closely spaced clips 40), the leaflets are too compliant, the leaflets are too stiff, or a relatively large tissue bridge has been previously created.

In the third scenario, the counter force helps to stabilize the valve clip 40 and leaflets such that the cutting loop can be pulled firmly against the leaflet edge. Once it is firmly pressed against the leaflet tissue, a first cut can be performed by pulling the cutting loop until it is positioned on the atrial side of the valve clip 40, and then the cutting loop can be cinched, and the final cut completed.

In the fourth scenario, removal of the engaged tissues may be necessary to facilitate the snare engagement and allow the snare to provide the counter force to stabilize the valve clip.

In the context of these scenarios, there are several embodiments and methods that can be used to produce this counter force towards/into the left ventricle 14, away from the left atrium 12.

In one example, the steerable catheter 180 used to deliver the cutting and retrieval catheter may be used to press on the valve leaflets 22, 24 and valve clip 40 to create counter force towards the left ventricle 14. For example, a distal edge around its distal opening can be positioned against the valve leaflets and/or the valve clip 40.

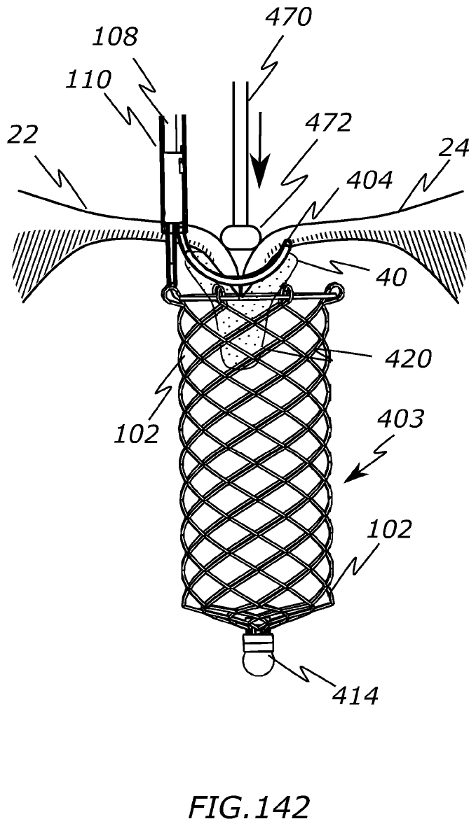

In another example, a pushing catheter 470 can be used to push on the valve clip 40 from the left atrial side of the valve clip 40 toward the left ventricle, as seen in FIG. 142. An elongated standard catheter or steerable catheter can be used, or the catheter 470 may further include a specialized distal end portion 472 to help press or engage the valve clip 40. For example, the distal end portion 472 may be selectively enlargeable (e.g., a balloon or expandable mesh structure) that can be expanded to better contact the valve clip 40. The distal end portion 472 may alternately or additionally have a blunted distal end. The distal end portion 472 may alternately or additionally be configured to mechanically affix to the valve clip 40 or even to leaflet tissue immediately adjacent to the valve clip 40. For example, the devices and mechanisms shown in FIGS. 70-100 may be used to affix to the valve clip 40 or to the adjacent tissue.

In any of these configurations, the catheter 470 can be pushed against the valve clip 40 from the left atrium 12 towards the left ventricle 14, and then the cutting loop can be positioned between the atrial side of the valve clip 40 and the leaflets 22, 24. Finally, the cutting loop (e.g., of any of the embodiments of this specification) can be activated and the cut through the leaflet tissue completed.

In yet another example, the valve clip 40 can be pulled from a location within the left ventricle 14, further into the left ventricle 14 to create the counter force, allowing the cutting loop to be positioned and to cut the tissue as previously described.

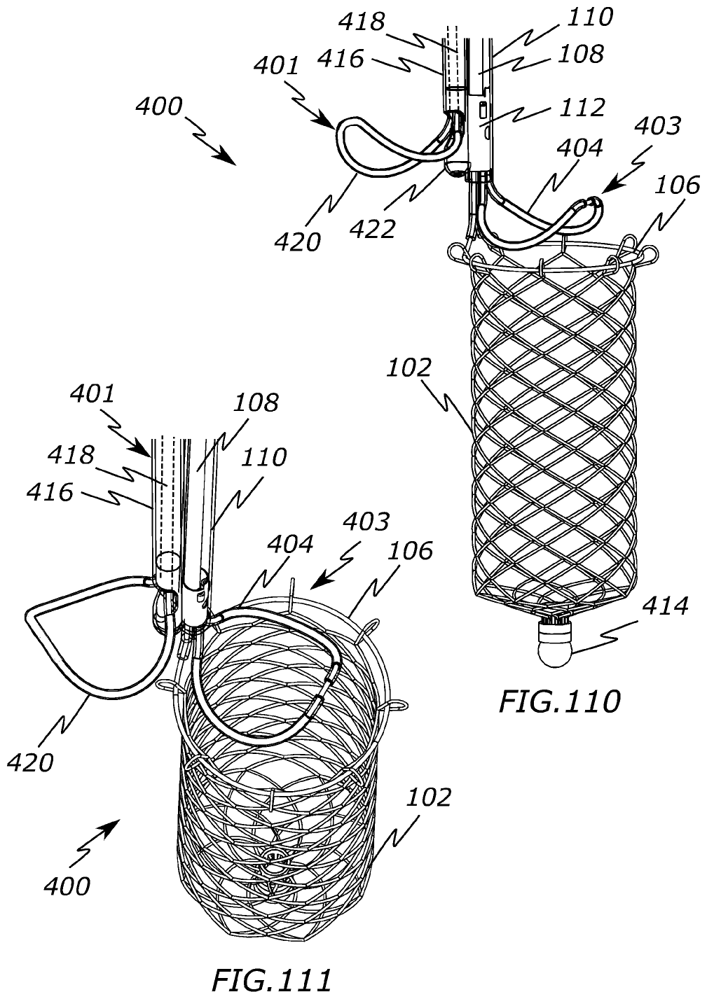

FIGS. 110 and 111 illustrate one embodiment of a removal system 400 that can be used to pull the valve clip 40 into the left ventricle 14 to create counter force, and includes both a cutting and capture catheter 403 and a separate snare catheter 401 (or alternately a separate cutting catheter and separate capture catheter). The snare catheter 401 can be used to first grasp the valve clip 40 and pull it further into the left ventricle 14, while the cutting and capture catheter 403 can be used to cut the valve leaflets 22, 24 and capture the valve clip 40. Note that this technique can be used on other valves, such as a tricuspid valve, as well.

The cutting and capture catheter 403 is generally similar to the previously embodiments and may include any of the variations previously discussed in that regard. For example, the cutting and capture catheter 403 may include a basket 102 and a cutting loop 404 connected to an elongated inner control member 108 that moves all of the components into and out of a tubular jacket or sheath 110.

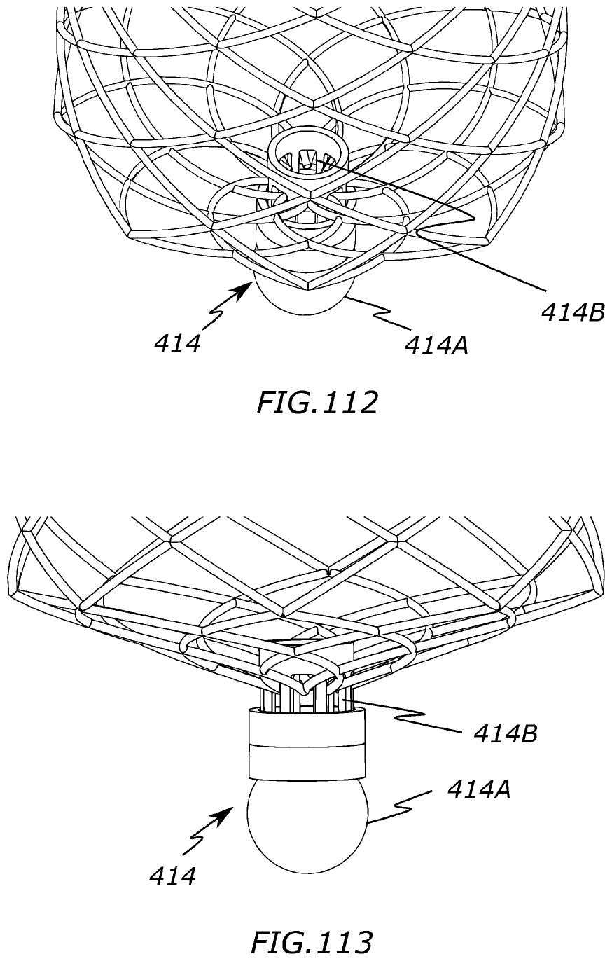

As best seen in FIGS. 112 and 113, the basket 102 may include a basket tip 414 at its distal end. The basket tip 414 can serve one or more of the following purposes. First, the basket tip 414 can connect to or contain the ends of the wires that make up the basket 102. In one example, the basket tip 414 includes a cylindrical wall portion with a plurality of apertures 414B through which the ends of the wires pass into, thereby preventing the wires ends from unraveling or from damaging the patient. The wires ends can be tied, welded, or adhered within the basket tip 414. Alternately, the ends of the wires can be welded or melted together with the basket tip 414 without the need for apertures 414B. The basket 102 can be woven with a single wire, in which case only two apertures 414B would be needed, or woven with a plurality of wires, in which a plurality of apertures 414B (e.g., two for each wire) may be needed.

Second, the basket tip 414 includes an atraumatic distal end shape 414A that helps prevent the basket tip 414 from damaging tissue within the patient. For example, the atraumatic distal end shape 414A can be spherical, rounded, oval, or conical. Again, this basket tip 414 may also be used with any of the other embodiments of this specification.

Returning to FIGS. 110 and 111, the cutting and capture catheter 403 further comprises a cutting element or loop 404 that can be structured and used similar to previously described embodiments. However, the present cutting loop 404 has a saddle shape, similar to cutting loop 350. That is, the cutting loop 404 has side portions that form a dip or curve distally downward and then proximally upward to form a valley shape. This shape can be desirable because, as the cutting loop 404 is proximally retracted inward into the outer sheath 110, the loop 404 may maintain a desirable cutting angle relative to the remaining elements of the catheter 403. For example, if the cutting loop 404 formed a perpendicular right angle relative to the inner control member 108, as that right angle joint is retracted into the outer sheath 110, it may tend to angle the loop 404 downward. In contrast, as the curved or saddle shape helps maintain the end of the loop 404 within a desired vertical range as it is retracted. This may be particularly helpful if the electrodes of the cutting loop 404 are located at the side of the loop opposite of the inner control member 108. Again, this cutting loop 404 can be used with any of the embodiments described in this specification.

The snare catheter 401 includes a snare loop 420 that is connected on or near the distal end of an inner control member 418 (which is similar to member 108) that moves longitudinally within a lumen of an outer tubular jacket or sheath 416. This allows the inner control member 418 to move the snare loop 420 into and out of the sheath 416. The outer tubular sheath 416 may have an opening at the distal bottom (i.e., at the axis of the sheath) similar to sheath 110 or may include a tip member 422 with an opening in its sidewall that extends into the interior lumen of the sheath 416 (e.g., a side opening that opens in a non-axial direction relative to the sheath). The outer sheath 416 may be elongated and therefore may have a length sufficient to enter a patient so that its distal end may reach an interior of the patient's heart (e.g., near a mitral valve). The lumen may open at or near the proximal end of the outer tubular sheath 416. The inner control member 418 may be a wire, tube or similar elongated member and may extend from the proximal end to the distal end of the outer tubular sheath 416.

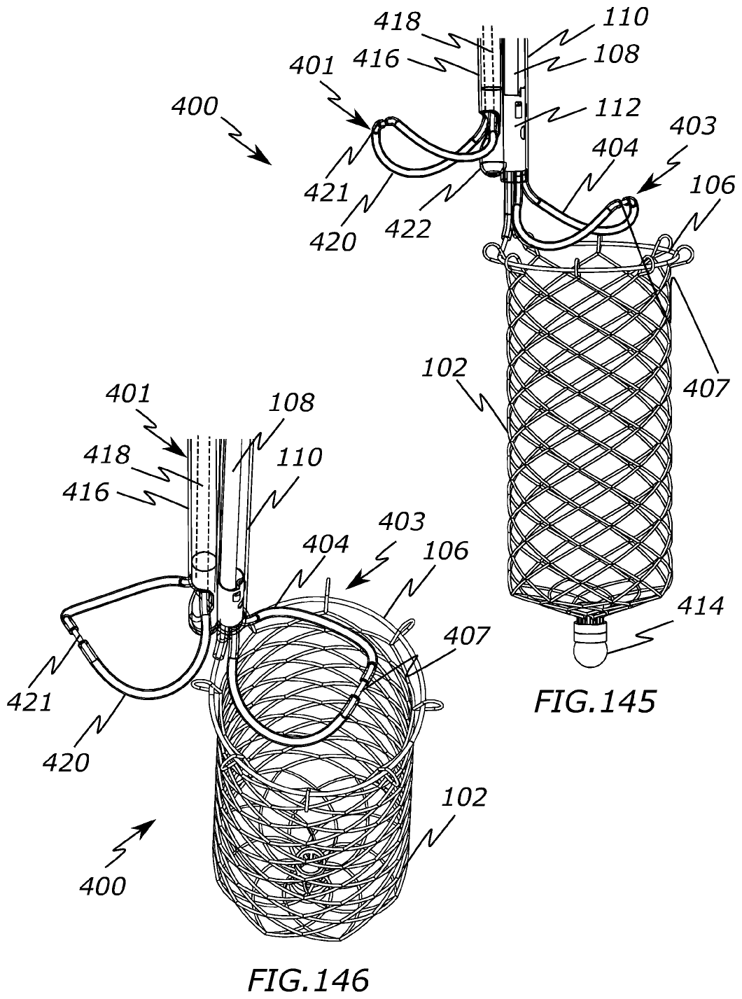

Optionally, the snare loop 420 may include a previously described cutting element (e.g., an RF electrode near its tip) that allows it to engage tissue surrounding the clip and at least partially cut some of this tissue to initially facilitate the capture and removal of the valve clip 40. Additionally cutting and capture procedures can then be performed. This initial removal of at least some of the engaged tissue may be necessary to facilitate the engagement of the snare loop 420 and allow the snare loop 420 to provide the counter force to stabilize the valve clip 40. For example, a channel or valley can be cut into the tissue surrounding the valve clip 40 which can then allow the snare loop 420 to more robustly engage the valve clip 40. One example configuration of this can be seen in FIGS. 145 and 146 which includes RF electrode 421 at the far end of the snare loop 420, in addition to RF electrode 407 at the far end of the cutting loop 404. However, any type of cutting element can be used. Alternately, the specified electrodes may instead be a radiopaque marker.

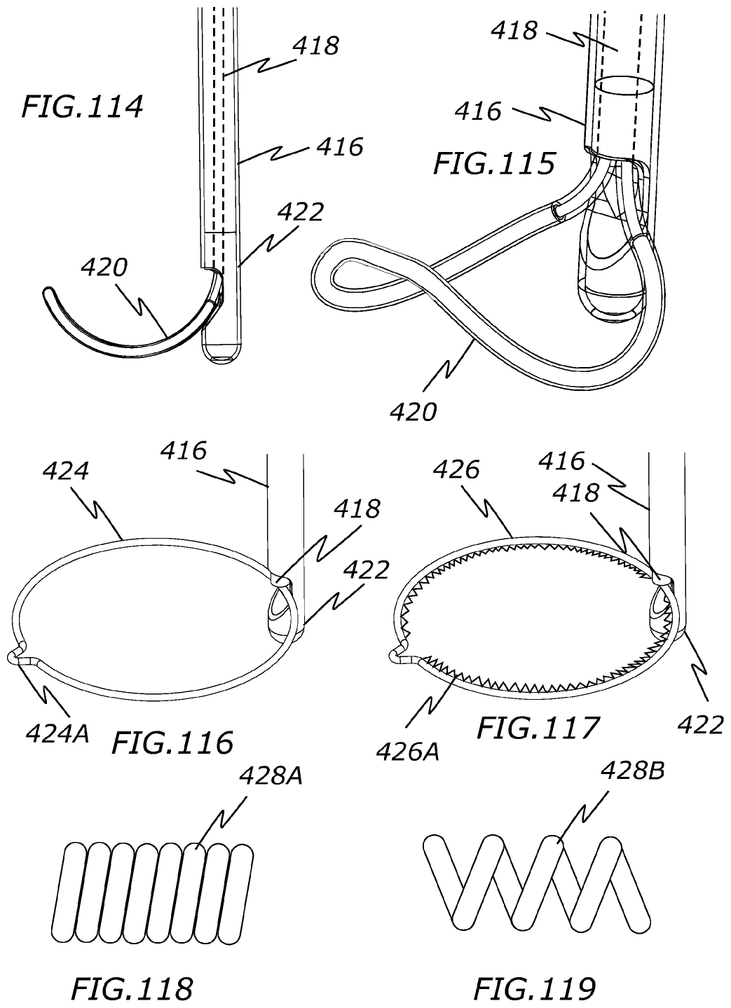

The snare loop 420 can have a variety of different shapes that are configured to grip a valve clip 40. For example, FIGS. 114 and 115 illustrate a “saddle-shaped” or arc-shaped loop similar to that of the cutting loop 404 (i.e., sides that curve distally downward and then back upward as they connect to each other to form an arc or concave shape). The curve shape can prevent the far end or tip of the snare loop 420 from moving distally downwards beyond the distal end of the distal end of the snare catheter 401. In other words, since the far end of the snare loop 420 is angled upwards when fully extended out of the catheter 401, as it is pulled in, the loop 420 may angle itself distally downwards, but since the far end is curved proximally, the distal angles of the loop 420 are not enough to move the loop 420 off o the valve clip 40. Put another way, the proximal curve of the loop 420 is sufficient that the far end of the loop does not position itself distally beyond a distal end of the snare catheter 401.

In another example seen in FIG. 116, a snare loop 424 can have a generally circular shape that may optionally include an indentation or curved segment 424A positioned opposite of the outer sheath 416 and extending radially outward from the circular shape. While only one notch or indentation 424A is illustrated, a plurality of segments 424A may also be positioned partially or completely around the snare loop 424.

The snare loop 424 has an imparted shape-memory shape such that it connects to the inner control member 418 at about 90 degrees. In some circumstances, as that 90-degree bend is pulled into the snare catheter 401, it may angle or deflect the loop 424 downward and could potentially move the loop 424 off the valve clip 40. In contrast, the saddle-shaped snare loop 420 has a distal/downward arc curve, and therefore as the loop 420 is pulled into the snare catheter 401, it helps maintain the original orientation of the loop 420 and thereby better maintains the loop 420 on the valve clip 40.

The snare loop 420 can further include features that enhance its grip on a valve clip 40. For example, a frictional coating or frictional sleeve can be placed over portions of or the entire snare loop, as seen with the snare loop 420 in FIGS. 114 and 115. In another example, a plurality of relatively small protrusions 426A (rounded bump, sharp teeth, or similar shapes) can be located on portions of or all of the inner radial surface/side of snare loop 426, as seen in FIG. 117. In another example, the snare loop can be composed of a wire coil in either a compressed configuration 428A (FIG. 118) or a longitudinally expanded configuration 428B (FIG. 119) which creates varying levels of texture and therefore increases the grasp of the loop on a valve clip 40. Again, these different loop shapes and frictional engagement features can be mixed and matched with each other and any embodiment in this specification.

While the snare catheter 401 and snare loop 420 is previously described as being used to grasp a valve clip 40, it can be used for other purposes as well. For example, it can be used to capture other tissues. It may be desirable to remove calcified nodules within the valve leaflets to facilitate placement of a valve. It may be used to capture and remove valve leaflet tissue to facilitate flow after a replacement valve is inserted. It may be used to remove chordae tendinea to facilitate motion of the valve. In that respect, the snare loop 420 can be used to grab such tissue while the cutting loop 404 is used to cut the tissue. However, it can also be helpful to include a cutting element (e.g., RF electrode) on the snare loop 420 to help cut such tissue first.

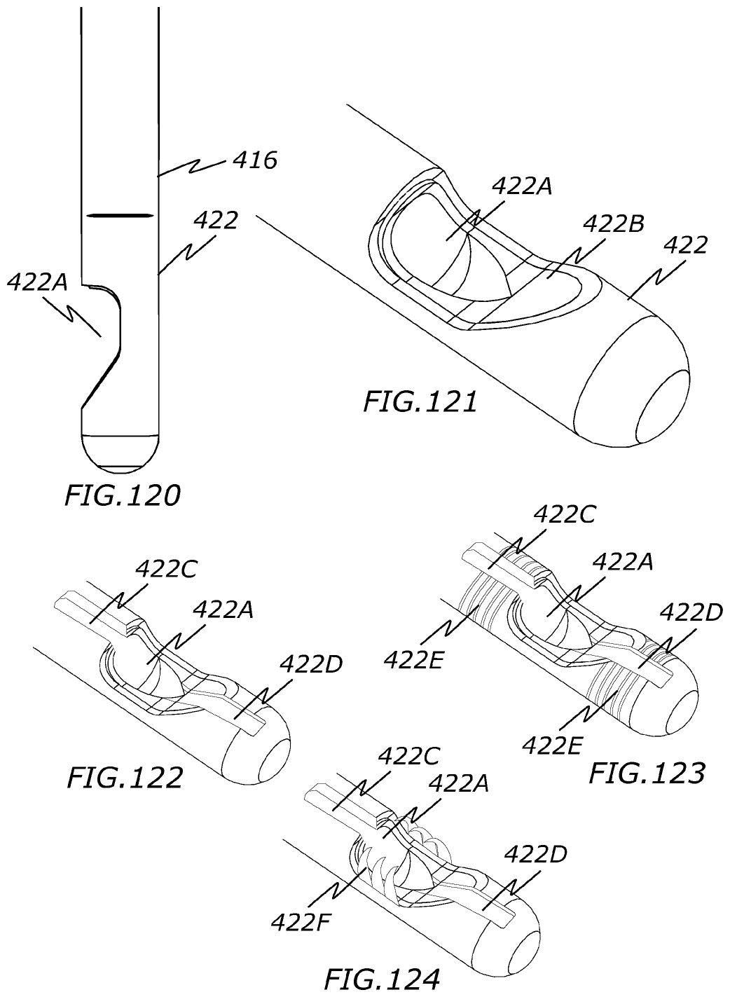

The snare catheter 401 may further include a tip member 422 at the distal end of the outer tubular sheath 416, as best seen in FIGS. 120 and 121. The tip member 420 can be configured to maintain the snare loop 420 in a desired angle or orientation relative to the outer tubular sheath 416 and the valve clip 40. Since valve clips 40 tend to have a generally conical or “V” shape, as shown earlier in this specification, a cinching snare loop may have a tendency to squeeze off of the valve clip 40 as it is tightened. This tendency to slip off may be increased if the snare loop 420 is positioned at a dramatic angle relative to a vertical axis of the valve clip 40. If the snare loop 420 was to exit the outer tubular member 416 from a distally facing opening (e.g., with no tip member 422), the angle of the snare loop 420 may increase relative to the axis as it is pulled into the outer tubular sheath 416, potentially slipping off the valve clip 40.

In contrast, the tip member 422 includes an opening 422A through its side wall, along with an angled or curved surface 422B that is configured to direct the snare loop 420 out of the opening 422A at a generally perpendicular angle relative to an axis of the outer tubular sheath 416 and axis of the valve clip 40.

Additionally, the tip member 422 can include features that help engage and/or create friction with the valve clip 40 so that when the snare loop 420 cinches the valve clip 40 against the tip member 422, the features help prevent the valve clip 40 from slipping out of the snare loop 420. In the example of FIGS. 120 and 121, the shape of the opening 422A may include relatively sharp or abrupt corners around it, which can help push into the valve clip 40 to retain its position.

In the example of FIG. 122, a proximal channel 422C and a distal channel 422D can extend from the main opening 422A to provide additional areas with sharp or abrupt corners that extend a greater longitudinal distance. In the example of FIG. 123, the tip member 422 can include a plurality of ridges or grooves 422E on the surface of the tip member 422, adjacent to the opening 422A and/or the channels 422C, 422D. These ridges or grooves 422E can be relatively straight and circumferentially oriented relative to the opening 422A and/or the channels 422C, 422D, or can have different patterns, such as waves or zig-zag patterns. In the example of FIG. 124, a plurality of spikes, hooks, or similar sharp shapes can extend from the side surface around the main opening 422A or other nearby locations. Any combination of these engagement features can be used together to provide surfaces that may better engage the valve clip 40 upon contact.

The snare catheter 401 may also include a handle 440 that is configured to retract the inner control member 418, and therefore the snare loop 420, in a controlled manner. Specifically, the handle 440 allows predetermined and limited amounts of force to be applied to the snare loop 420 while also providing the ability to lock the snare loop 420 position or force at the desired level. This allows the valve clip 40 to be grasped relatively firmly without enough force to break the catheter 401 and further allows this force to be maintained throughout the procedure without the necessity to hold portions of the handle 440 during most of the procedure.

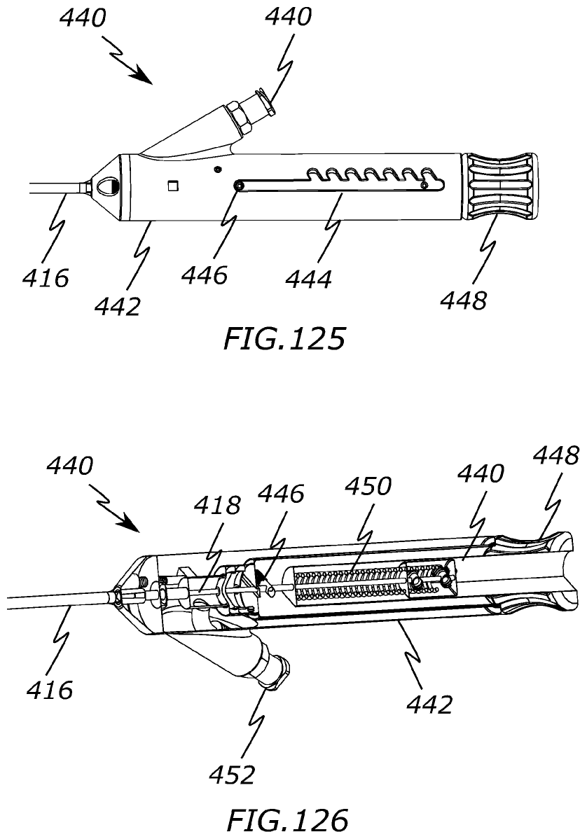

The handle 440 may have different possible mechanisms for controlling and locking the position/force of the snare loop 420. FIGS. 125 and 126 illustrate one such example mechanism having an outer housing 442 forming a generally cylindrical shape and a knob 448 located at a proximal end of the housing 442 and that is configured to be pulled proximally out from the housing 442 (i.e., to the right in the figures). Pulling the knob 448 out proximally also proximally pulls the inner control member 418, causing the snare loop 420 to retract proximally into the opening of the tip 422.

The force applied to the knob 448 can be limited or can increase in resistance as the knob 448 is pulled out by a spring 450 located in the housing 442. The spring 442 can be connected to either the inner control member 418 or a distal portion 440 of the knob 448, as well as to an interior of the housing 442 such that when the knob 448 is pulled proximally, the spring 450 either compresses or expands to generate increased resistance. Depending on the configuration of the spring 450 (e.g., size, amount of initial compression, spring constant, and similar aspects), the force applied by the spring 450 can be optimized to apply a constant predetermined tension throughout the length the knob 448 can be pulled, or alternately can apply increasing tension the further proximally the knob 448 is pulled.

The handle 440 may also include a locking mechanism that can lock the position of the inner control member 418 relative to the housing 442 and outer sheath 416. In the present example of FIGS. 125 and 126, the locking mechanism comprises a longitudinal slot 444 that extends along at least a portion of the length of the housing 442. A tracking peg 446 is fixed to the inner control member 418 and positioned at least partially within or through the slot 444. As the knob 448 is pulled proximally back, the tracking peg 446 moves along the length of the slot 444. When a desired position of the inner control member 418 has been reached, the knob 448 can be rotated so that the tracking peg 446 passes sideways relative to the length of the slot 444 into one of a plurality of slot branches. These slot branches are preferably shaped to retain the position of the tracking peg 446 and therefore the inner control member 444 when the user releases the knob 448. For example, the slot branches can be angled somewhat distally so that the distal force of the spring 450 maintains the tracking peg 446 within the slot branch, thereby releasably locking the inner control member 418 in place.

The handle may also 440 include a port 452 that is in communication with the interior of the handle housing 442 and outer sheath 416. This port 452 can be used for supplying saline, contrast, or similar fluids during a procedure.

The handle 440 may also include an electrical connection to the RF generator and to the snare loop 420 (if the snare loop includes an RF electrode or similar cutting element) to be used for supplying energy to the cutting snare 420 during a procedure. As previously described, in some circumstances it may be helpful for the snare loop 420 to include a cutting element to cut at least some tissue surrounding a valve clip 40 or other heart valve therapy so that the snare loop 420 can better engage the valve clip 40.

Generally, it may be desirable for both the cutting and capture catheter 403, and the snare catheter 401 to be included and delivered through the same delivery catheter (e.g., steerable catheter 180). However, the cutting and capture catheter 403, and the snare catheter 401 may also be separately delivered in independent delivery catheters or without any overlying catheter, depending on the specific procedure.

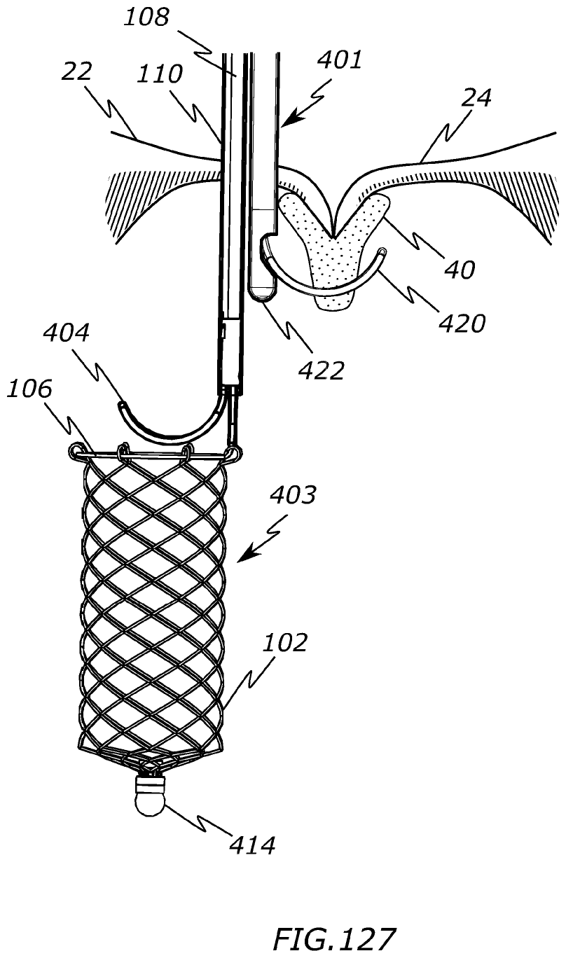

FIGS. 127-130 illustrate an example technique of using the removal system 400, including the cutting and capture catheter 403, and the snare catheter 401. While a specific removal system 401 is illustrated and discussed, variations on this procedure are also contemplated, including the use of the different embodiments described in this specification.

First, a delivery catheter (e.g., a steerable delivery catheter 180) containing the cutting and capture catheter 403, and the snare catheter 401 is advanced into the left atrium 12 of the patient's heart. It may be generally desirable for the tip of the catheter 180 to be advanced through the leaflets 22, 24 and into the left ventricle 14 initially. This may help prevent either the cutting and capture catheter 403 or the snare catheter 401 from becoming tangled in any chordae extending from or near the leaflets 22, 24. The basket tip 414 of the basket 102 may be positioned at or partially beyond the opening of the delivery catheter 180 to help create a generally smooth surface that will not “catch” or otherwise get stuck when passing through the leaflets 22, 24 or the nearby chordae.

Next, the cutting and capture catheter 403 and the snare catheter 401 are advanced out of the catheter 180 and into the left ventricle 14. Initially, the opening 422A of the tip member 422 is oriented towards the valve clip 40. If the snare loop 420 is not already deployed outward, the inner control member 418 is moved distally via the handle 440 to cause its deployment and expansion. The snare loop 420 is aligned with the valve clip 40 and then the snare catheter 401 is moved proximally so that the snare loop 420 surrounds the valve clip 40, as seen in FIG. 127.

During this time, the basket 102 and cutting loop 404 can be either be positioned in a different or opposite rotational orientation as the snare loop 420, and/or distally beyond the snare loop 420.

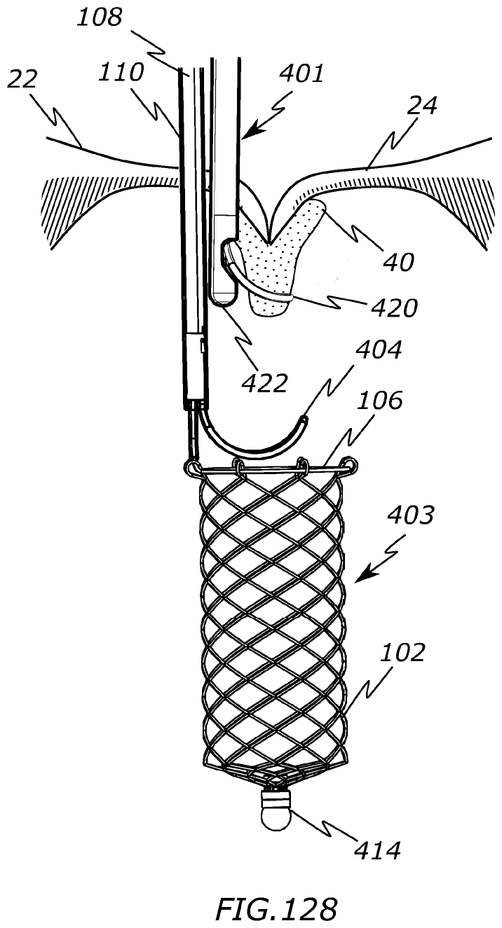

Referring to FIG. 128, the snare loop 420 is cinched or tightened around the valve clip 40 until the valve clip 40 is pressed against the tip member 422. This can be achieved by proximally retracting the inner control member 418 via the handle 440 (e.g., by pulling back the knob 448 and locking its position within the slot 444. If not already aligned, the cutting loop 404 and the basket 102 are vertically aligned with both the valve clip 40 and the snare catheter 401.

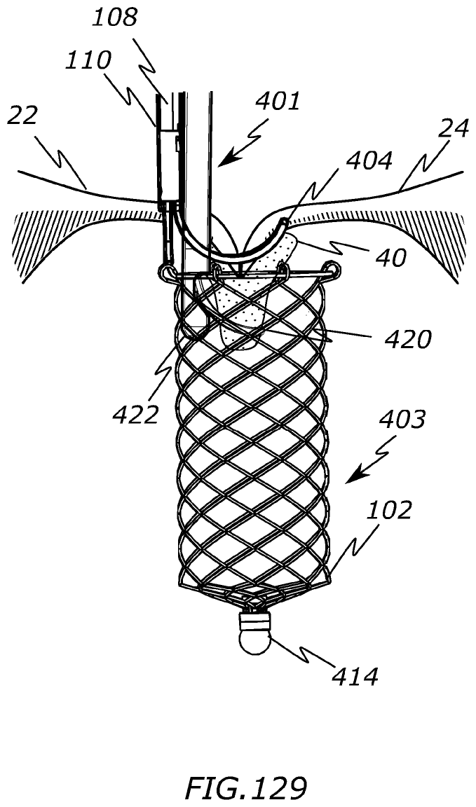

As seen in FIG. 129, the snare catheter 401 is advanced distally to create counter force to pull the valve clip 40 toward or further into the left ventricle 14. The cutting and capture catheter 403 is retracted proximally (either at the same time as the counter force is created or prior to it) so that the cutting loop 404 is positioned on the atrial side of the valve clip 40, between the clip 40 and the leaflets 22, 24. The basket 102 is positioned partially or nearly completely around the valve clip 40, as well as the distal portion of the snare catheter 401 (e.g., the snare loop 420 and distal tip 422).

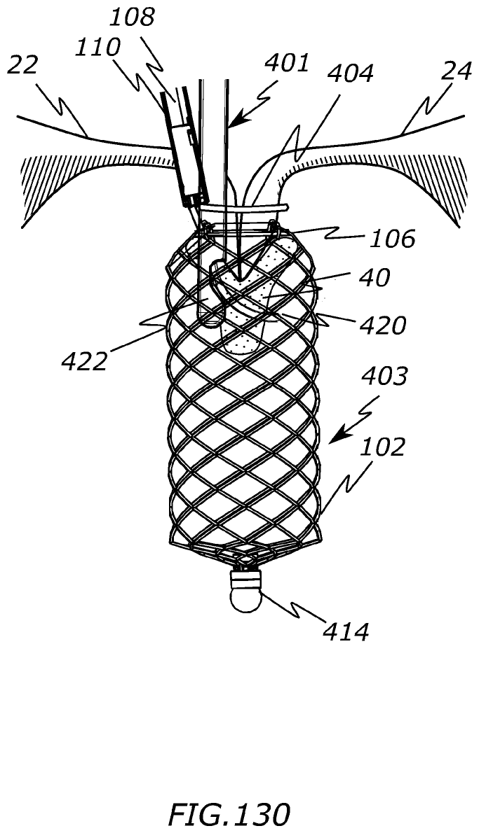

Referring to FIG. 130, both the cinching loop 106 and cutting loop 404 are cinched or reduced in size while the counter force from the snare catheter 401 is maintained. As previously discussed, this counter force allows good contact between the electrodes of the cutting loop 404 and the leaflet tissue. Hence, when the electrodes of the snare loop 404 are activated, a quick, clean cut can be performed through the tissue, causing the valve clip 40 to be completely removed and captured by the basket 102. Finally, the cutting and capture catheter 403 and the snare catheter 401 can be withdrawn at least partially back into the delivery catheter 180 and removed from the patient. The distal end/opening of the delivery catheter 180 may remain in the left atrium 12 or can be advanced back into the left ventricle 14 when the cutting and capture catheter 403 and the snare catheter 401 are retracted back within it.

In the scenario where the delivery catheter 180 passes back into the left ventricle 14 to then retract the cutting and capture catheter 403 and the snare catheter 401, there is a risk that the chords of the valve can inhibit the delivery catheter 180 from passing back through into the left ventricle 14. For example, the distal end of the delivery catheter 180 may not be tapered enough and therefore may “catch” on the chords.

One solution to this problem is to include a chord dilator that can be positioned partially out of the distal opening of the delivery catheter 180 when transitioning through the leaflets 22, 24 and chords. Such a chord dilator may include a tapered and/or angled distal surface and can be sized to radially occupy most or all of the opening of the delivery catheter 180.

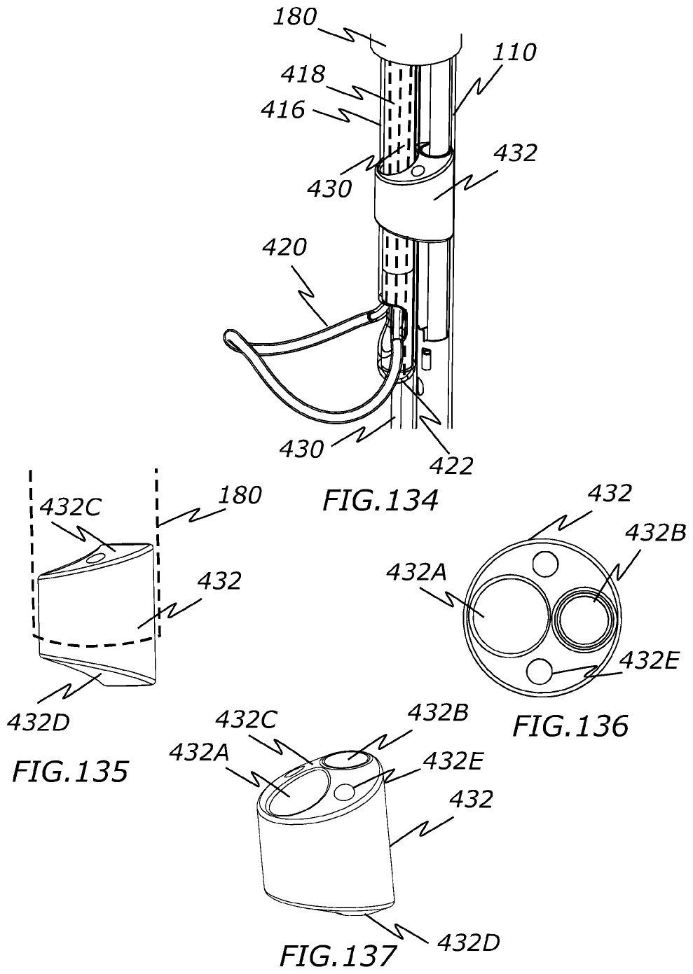

One example of a chord dilator 432 can be seen best in FIGS. 134-137. In this embodiment, the chord dilator 432 includes body having a bottom surface 432D that has a bias angle (i.e., an edge that forms an angle, such as about 45 degrees, relative to a vertical axis of the dilator 432). Additionally, the bottom surface 432D may be rounded or somewhat conical to help create a transition with the delivery catheter 180. Optionally, the chord dilator 432 may include a similar-shaped top surface 432C, which may be helpful in deflecting chords in both directions for either the mitral or tricuspid valve.

The chord dilator 432 includes two passages 432A and 432B; one passage 432A to accommodate the snare catheter 401 and another 432B to accommodate the cutting and capture catheter 403. Alternately, the chord dilator 432 may only have a single passage that accommodates only one of the snare catheter 401 or the cutting and capture catheter 403. In either case, the passages preferably allow their respective catheters to rotate within it during a procedure. Additionally, one of the passages may be a “C” shape (i.e., does not completely surround a catheter) such that it allows one of the catheters to decouple from the chord dilator 432. Optionally one, two, or more radiopaque markers 432E may also be included within the chord dilator 432. For example, the radiopaque markers 432E may be located on opposed circumferential sides of the chord dilator 432 and extend at least partially between the proximal and distal end of the chord dilator 432.

When the user desires to move the delivery catheter 180 into the left ventricle to capture the snare catheter 401 and/or the cutting and capture catheter 403, the snare catheter 401 and/or the cutting and capture catheter 403 are moved relative to the delivery catheter 180 so that they position the chord dilator 432 partially into the distal opening of the delivery catheter 180, similar to that seen in FIG. 135. Then, the delivery catheter 180, the snare catheter 401, and the cutting and capture catheter 403 are all distally advanced until the distal end of the delivery catheter 180 is positioned within the left ventricle 14.

Alternately, the chord dilator 432 may be configured only to connect the snare catheter 401 and the cutting and capture catheter 403, without any specific surfaces for chord dilation. In that respect, the chord dilator 432 may primarily be used to maintain the two catheters parallel to each other and not necessarily for chord dilation, which may be useful for achieving a desired alignment during a procedure.

The chord dilator 432 may alternately include three passages. For example, one passage 432A to accommodate the snare catheter 401, another 432B to accommodate the cutting and capture catheter 403, and another to facilitate a passage of a guidewire. The chord dilator 432 also can be used to facilitate the relative axial alignment of the snare, cutting loop, and basket. The location of the other members relative to the chord dilator 432 changes the length of the free member arms and hence controlling the distance between and angles the axis of the members

While the snare catheter 401 is previously described as being used to grasp a valve clip 40, it can be used for other purposes as well. For example, it can be used to grab or snare a guidewire or a Fogarty balloon during a procedure (e.g., during a transapical approach) to help pull these components through a target valve and prevent them from becoming tangled or stuck.

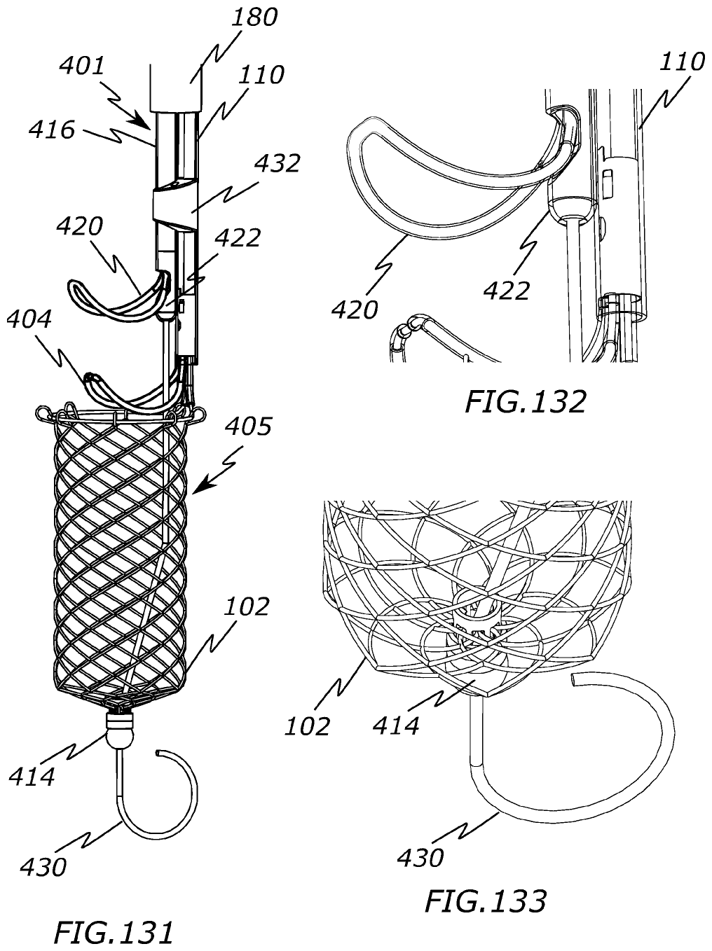

The removal system 400 is previously described as being delivered through an outer transeptal guide catheter 182 or similar catheter. However, the removal system 400 can be further configured to have a guidewire pathway or passage so that it can be delivered over a guidewire 430. The guidewire may have a more delicate force and stiffness transition to the tissues as the removal system is presented, ensuring safe introductions in to various anatomies. FIGS. 131-133 illustrate one example removal system 400 with such a guidewire passage. Specifically, the guidewire passage extends from a proximal opening into the outer sheath 416 of the snare catheter 401, through an opening in the distal tip member 422, through the basket 102, and finally through a passage in the basket tip 414.

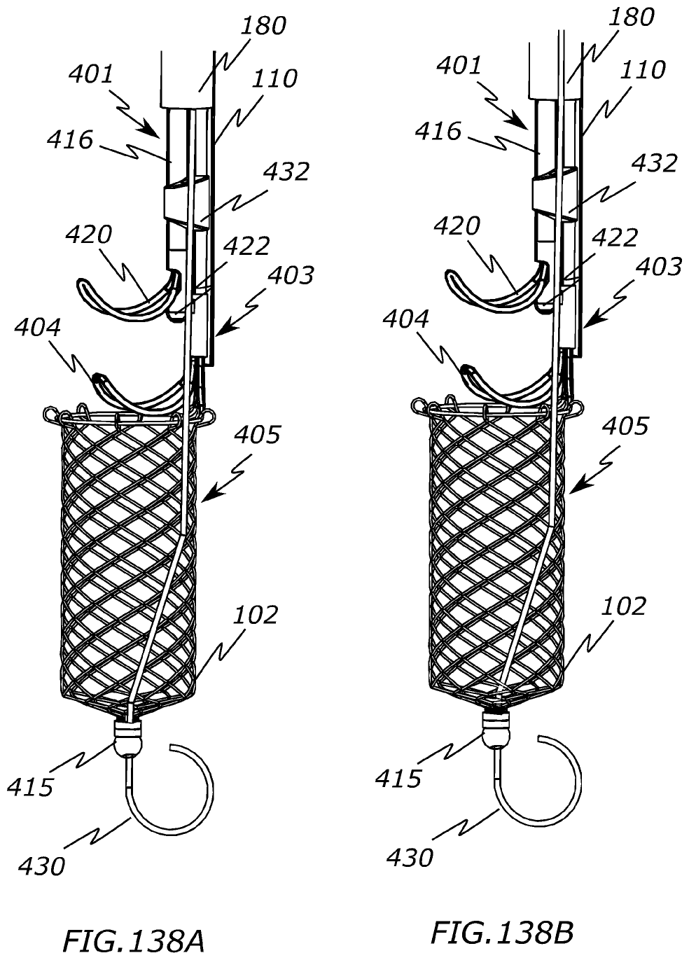

Other paths are also possible for the guidewire 430. For example, FIG. 138A illustrates a guidewire 430 that extends along the outside of the basket 405 and into a passage within the basket tip 415 that opens near its outer top and its bottom, thereby allowing the guidewire 430 to pass through. In this example, the guidewire 430 never passes into the interior of the basket 405. Alternately, in FIG. 138B, the guidewire 430 may pass through one of the cells of the basket 405, enter an interior opening to the passage of the basket tip 415, and extend distally out of the basket tip 415.

The guidewire 430 may proximally extend through only the steerable catheter 180, as seen in FIG. 138A, or may extend through passages in either the snare catheter 401 or the cutting and capture catheter 403. Alternately, the guidewire 430 may pass completely externally of the steerable catheter 180, snare catheter 401, or cutting and capture catheter 403 in the proximal direction (except for through the basket tip 415), as seen in FIG. 138B.

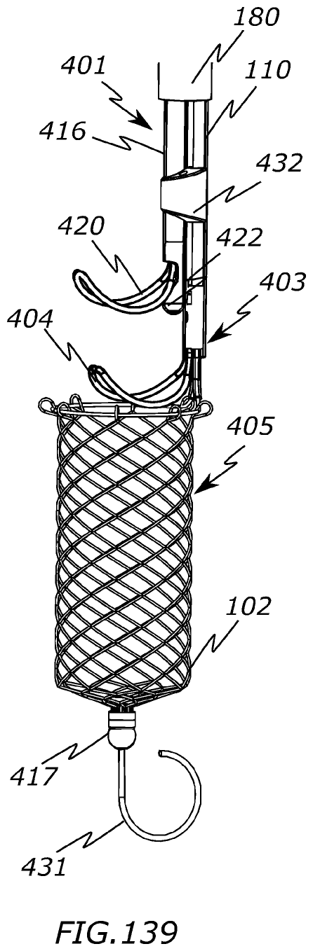

Alternately, as seen in FIG. 139, the basket tip 415 may include a guidewire-like pigtail or curved wire 431 attached and extending distally from the tip 417 to help pass through the valve and prevent damage to different heart structures.

The use of a guidewire 430 for delivery of the removal system 400 may facilitate crossing through a valve with smaller orifices, which is particularly common with valves that have multiple valve clips 40 implanted. Navigating over a guidewire 430 may also allow two valve clips 40 to be removed easier. For example, a catheter with a cutting loop 420 can be advanced over the guidewire 430 and used to cut the leaflet tissue between both valve clips 40, allowing them to spread apart and therefore allow for easier sequential capture via successive removal systems 400.

With regard to the sequential removal, this procedure may include placing a steerable catheter 180 within the left atrium, navigating a guidewire 430 through the target orifice of the valve, advancing a cutting and capture catheter 403 over the guidewire 430, removing the guidewire 430, cutting and extracting the valve clip 40 with the cutting and capture catheter 403, removing the cutting and capture catheter 403, again advancing the guidewire 430 through the target orifice of the valve, and repeating the removal process. The steerable catheter 180 may be left in place after removing the first valve clip 40 if it is sized large enough for complete removal of the valve clip 40; otherwise the steerable catheter 180 can be removed and a second steerable catheter 180 can be placed.

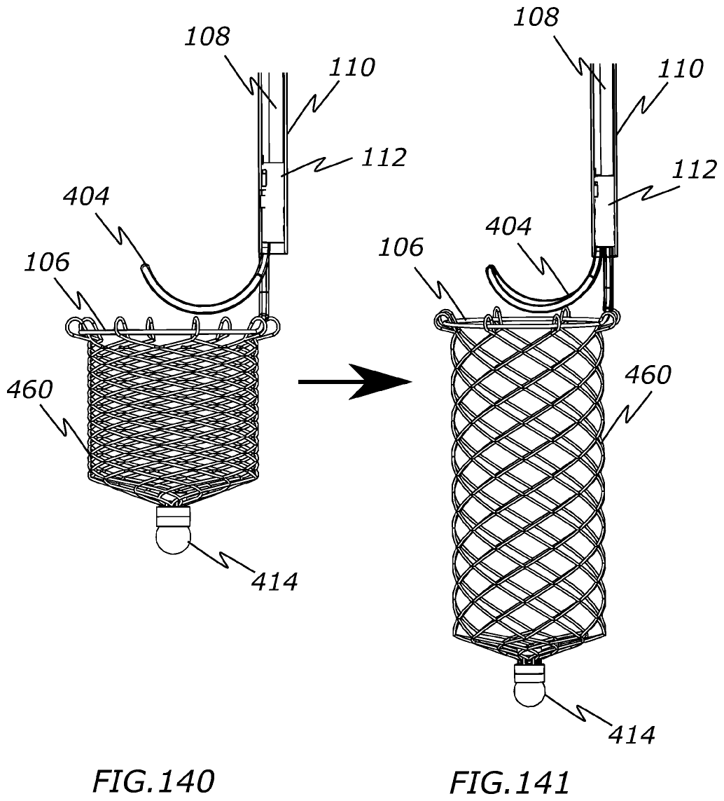

It may also be desirable in some embodiments and procedures for the cutting and capture catheter 403 to have a basket 460 that can expand or stretch from a longitudinally compressed configuration. This may allow for a basket 460 with a single size to accommodate different size valve clips 40, instead of needing several different sized baskets, and may also allow the valve clip 40 to be “tented” by the snare catheter 401. For example, FIG. 140 illustrates a basket 460 in a longitudinally compressed configuration and FIG. 141 illustrates the basket 460 in an expanded configuration.

The longitudinally stretchable functionality of the basket 460 can be achieved in several different ways. For example, the basket 460 can be braided or woven from one or more wires composed of a shape memory material (e.g., Nitinol), which is then heat set to the compressed configuration (FIG. 140). As an alternative or addition to heat setting, the braid pattern may also be configured to generally bias or at least facilitate the basket 460 in its compressed configuration. Alternately or additionally, the basket 460 may include longitudinal elastic tethers or an elastic outer cover that bias the basket 460 to its compressed configuration. Alternately, the wires of the basket 460 may be composed of an elastic material that allows for stretching. In another alternate embodiment, one or more of the struts/wires of the basket 460 may be coiled to form springs. In yet another alternate embodiment, a plurality of pull wires that connect to the basket 460 and to a proximal end of the device near the user may maintain the basket 460 in a compressed configuration and may be released proximally as needed to increase the diameter and/or length of the basket 460.

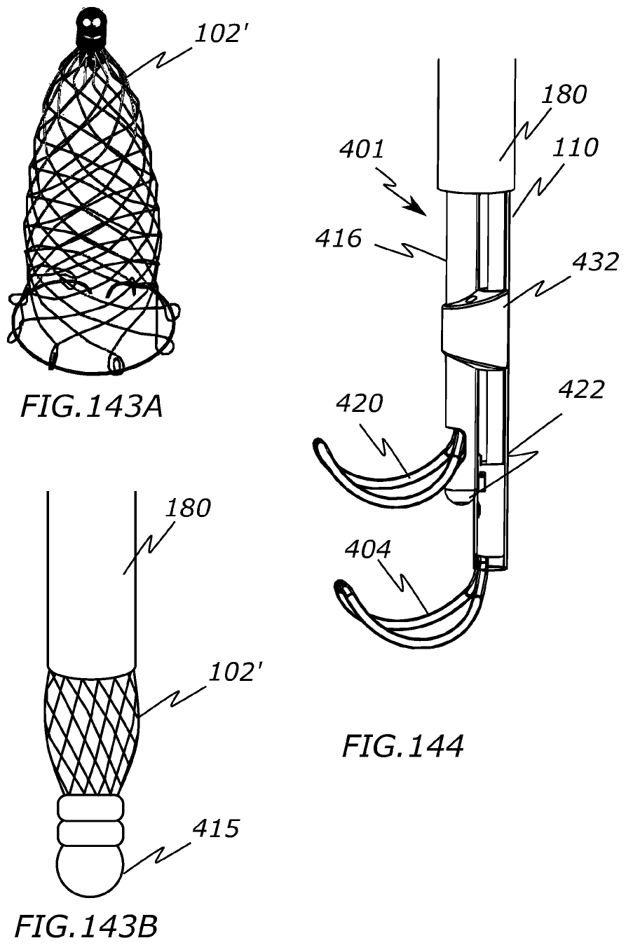

The baskets described in this specification (e.g., basket 102 or 460) have been shown to have a generally cylindrical expanded shape. However, other basket shapes are also possible. For example, FIG. 143A illustrates a basket 102′ that has a generally conical shape. In one example, the expanded basket shape tapers from about 30 mm to about 6 mm. When positioned partially out of the steerable catheter 180, as seen in FIG. 143B, the tapered shape creates a smooth, atraumatic shape without any large edges. This can help prevent the steerable catheter 180 from catching or getting stuck as it is crossing through the valve, leaflets, and chordae. This shape may also allow the basket to be loaded into the steerable catheter 180 or other catheters easier.

While the inclusion of the basket 102 is desirable for capturing a valve clip 40 or other heart valve therapy, it is also possible to eliminate the basket 102 from the cutting and capture catheter 403, as seen in FIG. 144. In such a device, the snare loop 420 (and possible other previously described clip engagement features) can be used to grab the valve clip 40 while the cutting loop 404 is proximally moved over the clip 40 and snare loop 420 and activated to cut the desired valve tissue. The snare loop 420 can maintain its hold on the valve clip 40 and proximally withdraw the clip 40 from the patient.

The present specification describes the use of one or more snare loops of a snare catheter that may be used in connection with the removal of various heart valve therapies (e.g., a valve clip). When using a snare loop with a cutting loop (e.g., in any of the techniques described in this specification), there may be a risk of the snare loop or other components of the snare catheter contacting or moving within close proximity of an active electrode on the cutting loop. Generally, any metal or conductive surfaces in close proximity with an active RF electrode may lead to direct coupling (e.g., arcing between the active electrode and the conductive object). This unintended arcing may be undesirable since it may damage portions of the snare catheter or snare loop, or may cause damage to unintended areas of tissue. Since existing snares are typically used for purposes in which they are not in close proximity to an RF cutting element, they often include exposed metal or other electrically conducting material.

Hence, it may be desirable for the snare loop and other snare catheter components that the loop is connected to be insulated and electrically isolated from other electrically conductive components. For example, electrical insulation may be included on one or all areas of the wire of the snare loop itself, the inner control member, the inner and/or outer surfaces of the outer sheath, and inner and/or outer surfaces of a distal tip of the outer sheath. For example, electrical insulation may be completely composed of non-conducting materials or may include one or more non-conducting layers or coatings, such as silicone, polyimide, dielectric coating, PTFE (e.g., a heat shrink tube), FEP, or similar biocompatible and non-conductive materials. In more specific examples, the snare loop may be composed of one or more nitinol wires that are insulated via silicone tubing, a dielectric coating, a braided polymer tubing (e.g., polyimide), a coiled polymer tubing (e.g., polyimide or polyolefin). This helps keep any conductive materials of the underlying loop wire (e.g., nitinol, 304 stainless steel, or similar shapeable materials) from conducting electrical current (e.g., RF current) during a procedure.

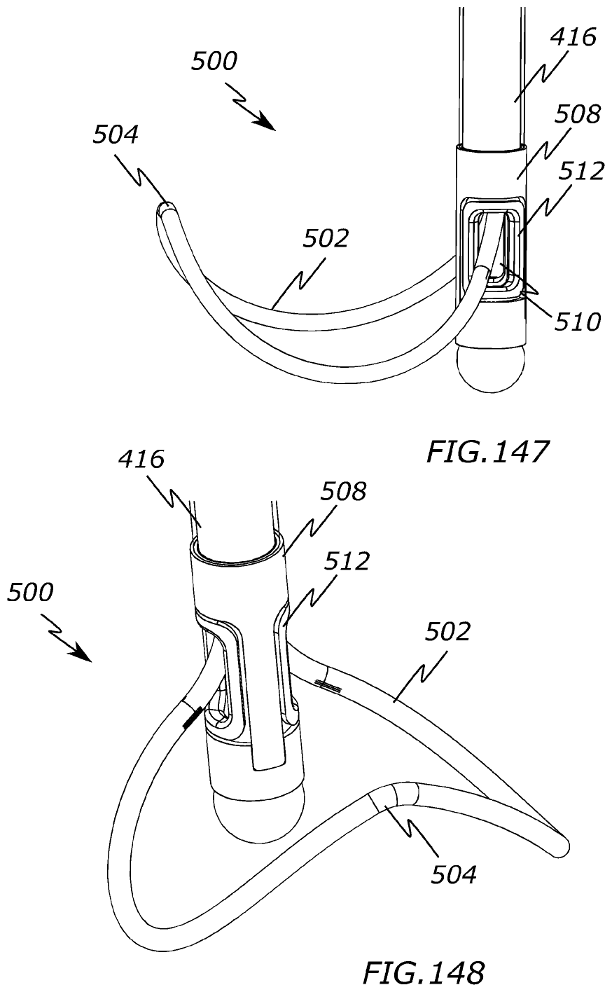

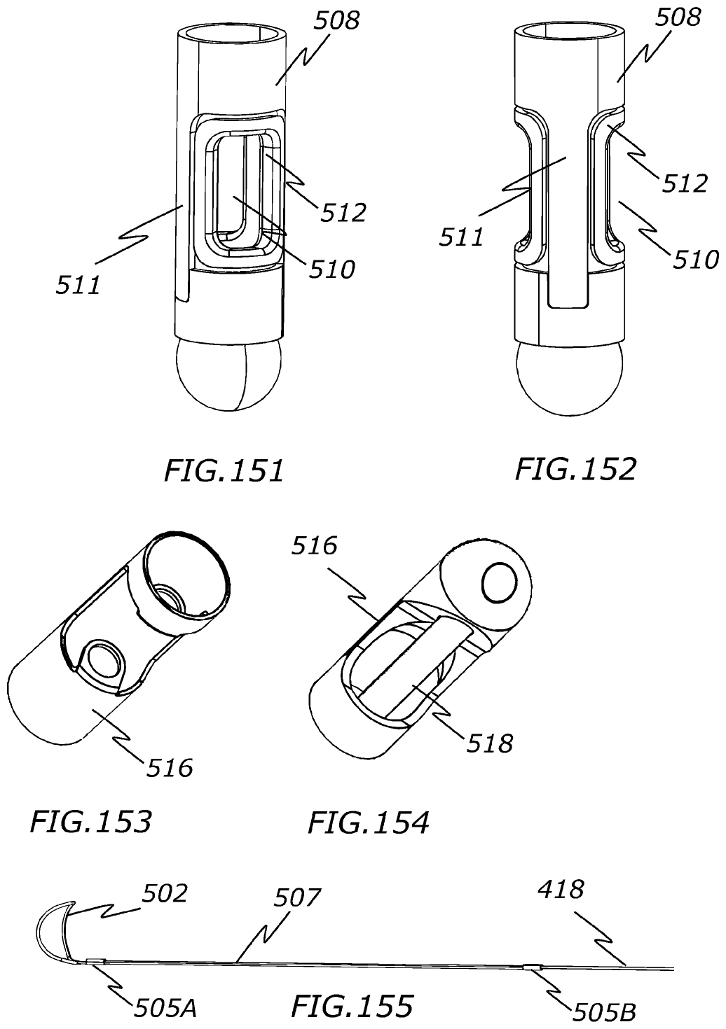

FIGS. 147-152 illustrate one example embodiment of a snare catheter 500 with electrical insulation that helps electrically isolate a snare loop 502 when in proximity to or contact with a cutting loop. This may prevent damage to the snare loop 502, among other components, and to non-target areas of tissue. The snare catheter 500 is generally similar to those previously described in this specification in use in structure. For example, the snare loop 502 is located at a distal portion of the snare catheter 500 and may be connected to an inner control member 418 to allow a user to increase or decrease the size of the loop 502 outside of the outer tubular jacket or sheath 416 and distal tip member 508. This size adjustment may be achieved by the user via a proximal handle 514 having a movable interface element (e.g., a slidable tab) that longitudinally moves the inner control member 418 within the outer tubular jacket 416.

In the present example, the snare loop 502 has a saddle shape, as previously described, but can be any shape or configuration described elsewhere in this specification. The loop may include features that help visualization under medical imaging. For example, some or all of the loop may include radiopaque material. In one specific example, discrete radiopaque markers 504 may be included at the tip of the loop and/or at other side locations (e.g., radiopaque tubes or sleeves placed over nitinol wire forming the loop 502). In another specific example, radiopaque wires may be braided, twisted, coiled, or otherwise incorporated with nitinol wires to form the loop 502.

As previously described, the snare loop may include a coating, layer, or tube positioned over some or all of its underlying snare wire(s) (e.g., shape memory material such as nitinol, stainless steel, and radiopaque materials described in this specification). This coating/layer/tube may include silicone, polyimide, dielectric material (e.g., Polyolefin), PTFE (e.g., a heat shrink tube), FEP, or similar biocompatible and non-conductive materials. More specifically, the coating of the underlying wire of the snare loop 502 may be composed of silicone tubing, a dielectric coating, a braided polymer tubing (e.g., polyimide), a coiled polymer tubing (e.g., polyimide). The inner control member 418 and its connection points to the snare loop 502 may also be partially or fully insulated with similar materials.

The snare catheter 500 may also include a distal tip member 508 that includes an open region into one or more openings 510 (e.g., one or two openings) that portions of the snare loop 502 exit from. These openings 510 may be located at the sidewall of the distal tip member 508 as seen in the figures, or a single opening may alternatively be located on the distal, axial (did you mean distal?) end. An atraumatic distal end may also be included, such as the rounded or spherical tip of the distal tip member 508 shown in the figures.

The distal tip member 508 (i.e., its structural material) may be composed of a non-conductive material, such as a polymer. Alternatively, the distal tip member 508 may be composed of a conductive metal that is at least partially coated or fully coated with a non-conductive material, such as those previously described.

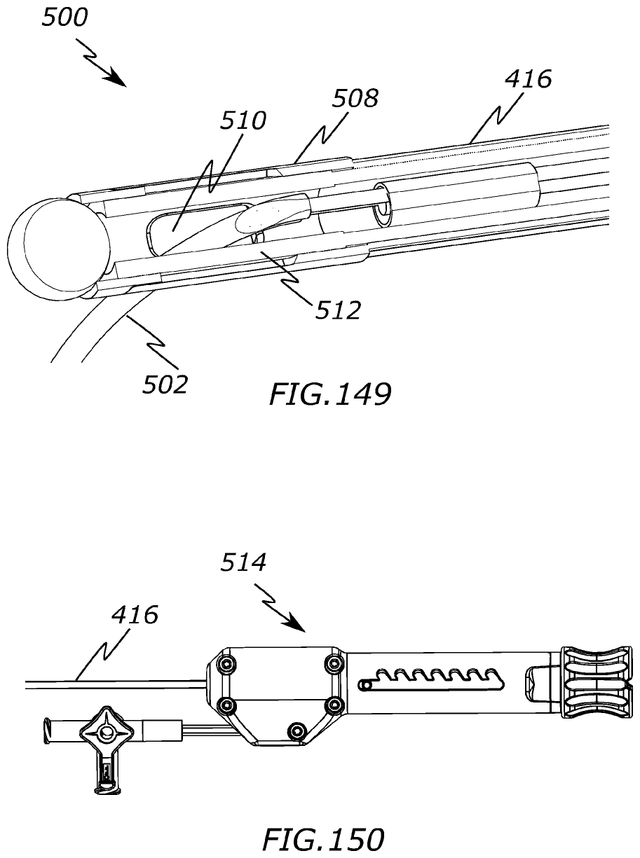

The distal tip member 508 may also include molding or an insert 512 at least partially within a cavity or passage of the distal tip member 508 (FIG. 149), that may be composed of non-conductive material, such as those previously described. Additionally, the insert 512 may be positioned over an edge of each of the openings to help prevent any sharp edges around the openings 510 from scraping off any insulation layer from the snare loop 502 while being moved into or out of the distal tip member 508. For example, the insert 512 may form a rounded, curved, or blunted shape over and around the edges of the openings 510. Alternatively, the insert 512 may not be needed if the distal tip member 508 is otherwise shaped to have rounded or blunted edges around the openings 510 and is composed entirely of non-conductive material or has an outer non-conductive coating.

As previously discussed elsewhere in this specification, it may be helpful for the snare loop 502 to move out of the snare catheter 500 (e.g., from the distal tip member 508) in a generally sideways trajectory relative to an axis of the snare catheter 500. The side exit point of the snare loop 502 and its generally concave shape opening proximally may help maintain a snared heart valve therapy device at a relatively constant proximal/distal position relative to the snare catheter 500 as the snare loop 502 is tightened and the heart valve therapy device is brought closer to the distal tip. Maintaining this constant proximal/distal position may be important for grabbing the heart valve therapy device from a more “atrial” position (i.e., close to the valve leaflets). Generally, the more “atrial” the position of the snare loop 502 on the heart valve therapy device, the more stable and secure the grasp of the snare loop 502 is throughout a procedure.

Unlike the previously described distal tip member 422 in FIG. 115, the distal tip member 508 includes a vertical or axially parallel bar 511 or structural feature separating the two openings 510. This vertical bar 511 may help prevent the snare loop 502 from being fully pulled into the distal tip member 508 and therefore damaging the snare loop 502 (e.g., deforming the loop 502 and/or removing part of its insulating coating). The use of relatively larger diameter wire to form the snare loop 502 and the need to maintain a relatively small diameter/profile of the snare catheter as a whole may result in the deformation or damage to the snare loop wire if fully withdrawn into the distal tip member 508, depending on the size of these components.

FIGS. 153 and 154 illustrate an alternative embodiment of the distal tip member 516 that has a single opening, a lower angled surface, and a cylindrical middle bar 518 that can be inserted into the distal tip member 516 during manufacturing. Again, this middle bar 518 may help prevent the snare loop 502 from being fully pulled into the distal tip member 516 and thereby damaged. All of these components may be either entirely composed of non-conductive material or may be coated with non-conductive material.

Several factors can apply force to a snare loop during a procedure which may cause it to deflect and therefore increase the difficulty of snaring a heart valve therapy device. For example, the patient's heartbeat, blood flow, the presence of chordae, movement of the leaflets, and movement of the heart valve therapy device itself may cause unwanted deflection, rotation, twisting, or similar movement of the snare loop. In that respect, it may be desirable for the snare loop to have a stiffness that reduces deflection while also limiting damage or deflection when the snare loop is withdrawn into the snare catheter. More specifically, it can be desirable for the snare loop to have a stiffness that reduces or substantially prevents deflection, rotation, or twisting around an axis of the snare catheter (e.g., proximally or distally bending relative to the snare catheter axis) or twisting/torsion of the snare loop relative to the axis of the snare catheter. For example, the snare loop may be stiff enough to prevent deflection from at least blood flow through a heart. In another example, the snare loop may be stiff enough to deflect chordae tendon and or to push heart valve clip and valve tissue around to optimize the ability to capture the heart valve clip.

Note, the following examples describe forces applied to the snare loop in terms of mass (e.g., grams) applied under standard gravity. One of skill in the art would be capable of converting these numbers to force values. The following examples assume that the underlying wire diameter of the snare loop is within an inclusive range of about 0.001 inch to about 0.030 inch. The following examples assume that the shape of the snare loop has a working diameter (i.e., an unconstrained expanded diameter) within an inclusive range of about 0.100 inch to 1.5 inches. In one example, the snare loop is constructed in a manner that limits deflection of a distal tip of the snare loop (proximally or distally) to an inclusive range of about 0 mm and 10 mm when force is applied to snare loop (e.g., the distal tip) within an inclusive range of about 0 grams to 3 grams. In a more specific example, the snare loop is constructed in a manner that limits deflection of a distal tip of the snare loop (proximally or distally) to an inclusive range of about 0 mm and 5 mm when force is applied to snare loop (e.g., the distal tip) within an inclusive range of about 0 grams to 2 grams. In another more specific example, the snare loop is constructed in a manner that limits deflection of a distal tip of the snare loop (proximally or distally) to an inclusive range of about 0 mm and 2 mm when force is applied to snare loop (e.g., the distal tip) within an inclusive range of about 0 grams to 2 grams. In a more specific example, the snare loop is constructed in a manner that limits deflection of a distal tip of the snare loop (proximally or distally) to an inclusive range of about 0 mm and 2 mm when force is applied to snare loop (e.g., the distal tip) within an inclusive range of about 0 grams to 2 grams. In yet another specific example, the snare loop is constructed in a manner in which its distal tip deflects about 1 mm with about 0.75 grams of weight, about 1 mm with about 1.125 grams of weight, about 2 mm with about 1.5 grams of weight, and about 2 mm with about 1.875 grams of weight (“about” indicating a +/− range of about 15%). Note, the stiffness, underlying wire size, and working loop diameter of the snare loop examples above may also apply to examples of a cutting loop and of a cinching loop of a capture basket described elsewhere in this specification.

Generally, there are several different ways to construct a snare loop that is relatively stiffer and therefore deflects or twists less during a procedure (e.g., deflects as noted in the example ranges above). In one example, the snare loop may be formed from a solid wire (e.g., nitinol) as opposed to a braided/stranded/twisted wire. In another example, the snare loop may be formed from a wire with an outer coating (e.g., metal or polymer coating). A solid or braided/stranded/twisted nitinol wire may be coated with stainless steel, a polymer, or even radiopaque materials such as gold, platinum, or tantalum. Hence, drawn filled tubing (DFT) may also be used for the snare loop. In another example, multiple wires positioned adjacent to each other are possible. In another example, braid reinforced tubing or wire (i.e., tubing or wire with a braided wire layer on the outside) is also possible. In another example, a square or rectangular cross sectional shape is possible.

FIG. 155 illustrates one example of a snare loop 502 connected to an inner control member 418 (e.g., a core wire) which may provide a desired level of deflection resistance or stiffness during a procedure. The snare loop 502 may be composed of a solid shape memory wire (e.g., nitinol wire) having a diameter within an inclusive range of about 0.001 inch to 0.03 inch. This nitinol wire may optionally include one or more radiopaque markers (e.g., a radiopaque coil or sleeve at the tip or sides, or a radiopaque coating on the nitinol wire). An electrically insulative coating may also be located over both the shape memory wire and the radiopaque marker(s).

The proximal ends of the shape memory wire may be connected to the inner control member 418 via a distal crimp 505A (e.g., a metal sleeve crimped over the other components). The distal region of the inner control member 418 may include a polymer heat shrink tube 507 that may add some stiffness but also may electrically insulate the inner control member 418 from use of a cutting loop nearby. The heat shrink tube 507 may be further connected to the inner control member 418 via the distal crimp 505A and a proximal crimp 505B.

The snare loop may also include a pointed distal end to help navigate through chordae during a procedure. For example, the snare loop 502 of FIG. 148 includes a rounded point, but more aggressively angled point may also be possible. For example, it may be desirable to form a distal point in which two regions of the wire of the snare loop come together at an angle (rounded or abrupt) within an inclusive range of about 80 degrees to about 130 degrees. Alternately, the snare loop may have a protruding feature (e.g., a sphere, cube, triangular, pyramid, or elongate shape) that extends outward from a distal portion of the distal tip.

Cutting loops have been discussed in this specification for cutting tissue within a vascular system of a patient and specifically, for example, during a procedure for removing heart valve therapy (note that the term “cutting loop” is defined broadly in this specification to include full loops, partial loops, or any elongated curved shapes). Additionally, the cutting loops of this specification may be used to cut chordae and/or heart/valve tissue. The cutting loops may be used alone, as part of an individual cutting loop catheter or with other devices described in this specification, such as with a capture basket (e.g., combined as a single tool or as separate tools used together). Additionally, the cutting loops of this specification may have the same size and particularly stiffness levels as described previously for snare loops, such that deflection or twisting of the cutting loop is minimized or prevented at least based on blood flow pressure within a heart.

In some uses, it may be desirable to deliver relatively higher RF power to the cutting loop to better cut target tissue (e.g., power within an inclusive range of about 5 watts to 1600 watts). However, such higher RF power may, in some circumstances (e.g., during partial or full exposure to blood and not fully embedded in tissue), damage portions of the cutting loop because they create higher temperatures. Since the cutting loop may be primarily composed of a shape memory material, such as nitinol, these higher temperatures may change any heat-set shape imparted to the material, among other damage. Such damage may hinder or prevent further cutting and may increase the difficulty of removing the cutting loop from the patient. Hence, it may be desirable to improve the delivery of higher RF power by including certain materials and/or shapes into the electrodes. These techniques can be used with any of the cutting loop or cutting devices described in this specification.

Turning first to materials, it may be desirable that an electrode of a cutting loop be composed of a material that can withstand higher temperatures before being damaged. In such cases, materials without shape memory properties may be used, such as stainless steel or similar metals. For example, the cutting loop may be formed of one or more wire segments of shape memory wire (e.g., nitinol) that are connected to adjacent segments of non-shape memory wire (e.g., stainless steel). Alternatively, a non-shape memory tube or sleeve may be connected (e.g., swaged, welded, adhered, etc.) to a shape memory wire (e.g., nitinol) to form an electrode. The shape memory wire segments may include an electrically insulating outer layer so that only the non-shape memory portions are exposed to form the electrode(s).

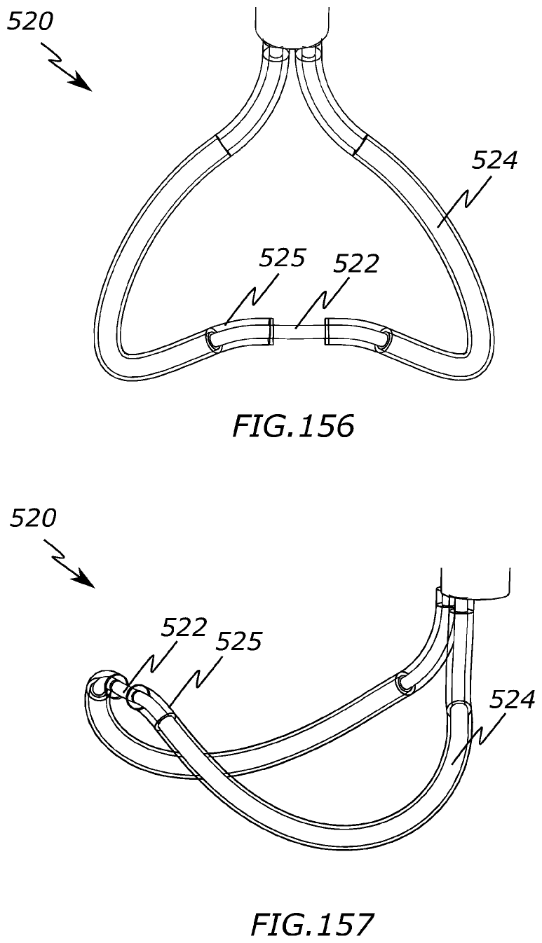

FIGS. 156 and 157 illustrate one example embodiment of this configuration in which a cutting loop 520 having primary loop segments 524 formed of portions of shape memory wire and one or more electrodes 522 of partial or full non-shape memory material (e.g., a stainless-steel wire segment or sleeve). An electrically insulating layer 525 may be disposed over at least the primary loop segments 524 and optionally over portions of the electrode(s) 522 if needed. Again, while only one electrode 522 is shown, multiple electrodes and segments are possible.

Turning next to the geometry of the cutting loop electrodes, the shape of the electrode may also be helpful reducing damage to the components of the cutting loop, such as shape memory wire and electrical insulation. The Applicant has found that electrodes with points, edges, raised surfaces, or similar features may concentrate areas of electrical dissipation. In that regard, electrodes with such features may require less RF power than electrodes without such features and therefore may help keep the temperature of non-electrode portions of the loop at a more desirable level to avoid damage. Hence, such electrodes may have a larger diameter relative to adjacent primary loop segments, including shapes specified herein. Example electrode shapes include cylindrical, spherical, cubic, curved, ridged, and similar variations and combinations (e.g., cylindrical with a ridge). These electrode shapes may be used with any of the other electrode or cutting loop features described in this specification. Additionally, if multiple electrodes are included, all of the electrodes may be the same shapes or different shapes. Additionally, the shapes may evenly or uniformly extend around the circumference of the electrode or may only extend from part of the electrode (e.g., from the interior or exterior of the loop).

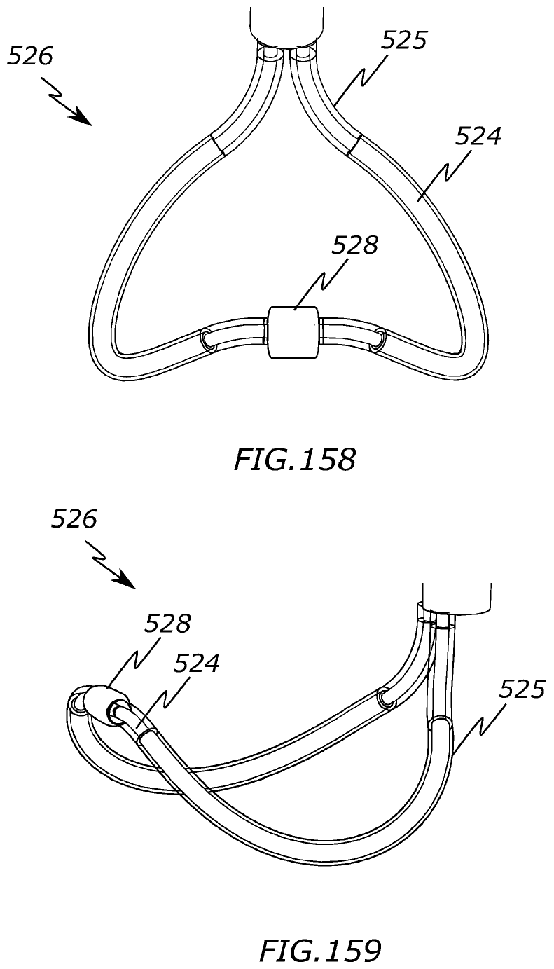

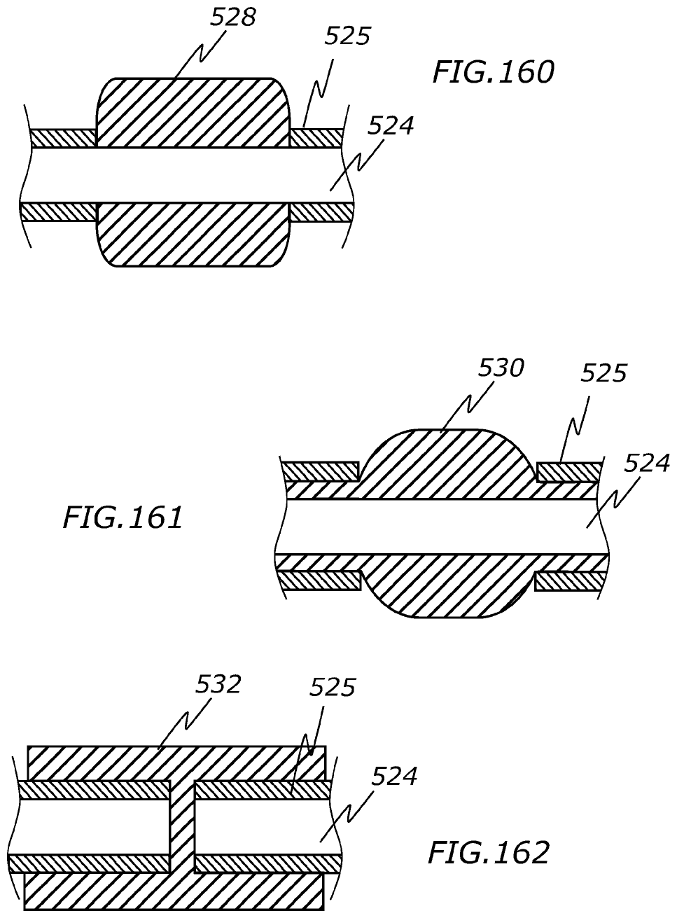

FIGS. 158-160 illustrate one example of a cutting loop 526 which is generally similar to previously described cutting loop 520, but has an electrode 528 having a generally cylindrically shape that is thicker or raised above the surrounding portions of the primary loop segments 524 and insulation layer 525. The electrode 528 may be composed of a discrete segment (e.g., stainless steel) connected to the wires of the primary loop segments 524, or may be composed of a sleeve that partially or fully surrounds the underlying shape memory wire of the cutting loop 526, as seen in the cross sectional view of FIG. 160.

FIG. 161 illustrates another example shape of an electrode 530 with a shape that is relatively spherical or rounded, as well as elevated relative to adjacent regions of the cutting loop.

FIG. 162 illustrates an example of a generally cylindrical electrode 532 that connects to two adjacent ends of the primary loop segments 524. To enhance the connection to the primary loop segments 524, the electrode 532 may form cylindrical wire shape cavities on either end sized such that the primary loop segment 524 and optionally the insulating layer 525 may fit and be connected within (e.g., adhesive, welding, etc.).

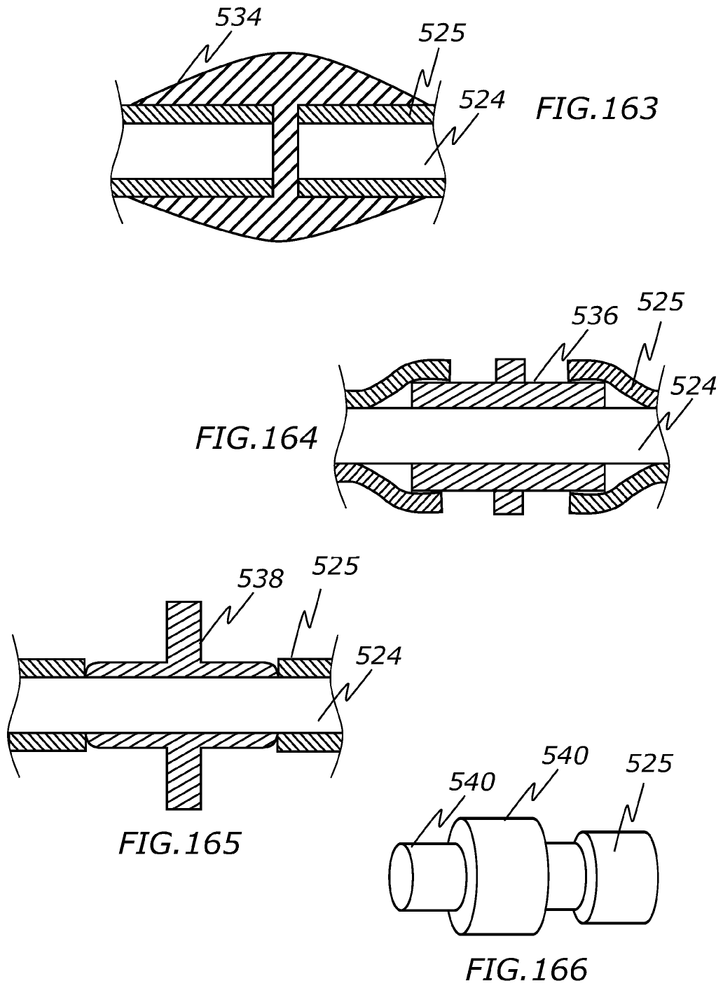

FIG. 163 illustrates an electrode 534 is similar to cylindrical electrode 532, but its outer surface has an elevated, rounded middle relative to its ends. In other words, the electrode 534 tapers to a smaller radial diameter from its middle towards each end.

FIG. 164 illustrates an electrode 536 that has a generally cylindrical shape with a middle ridge extending partially or fully around the electrode 536. In the present example, the electrode 536 is connected around the primary loop segment 524 and the insulating layer is disposed over portions of one or more ends of the electrode 536. Alternatively, the insulation layer 525 may not be disposed over the electrode, as seen with electrode 538 in FIG. 165.

FIG. 166 illustrates an electrode 540 that is similar to electrodes 536 and 538, but has a relatively wider cylindrical middle ridge.

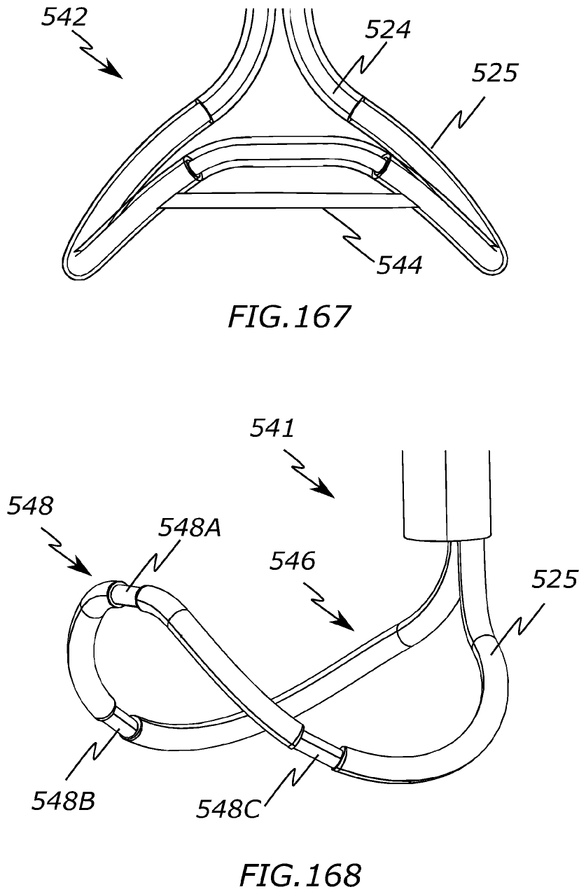

In another example, the electrode may be a wire that is spaced apart from the primary loop segment 524 and insulation 525 along most of its length. FIG. 167 illustrates one such example of a cutting loop 542 with a wire electrode 544 that is connected at each of its ends to the cutting loop 542 so that some or all of the wire electrode 544 is spaced apart from the cutting loop 542, thereby creating space or a gap. In the present example, the ends of the wire electrode 544 are connected at locations on either side of a middle end or point of the cutting loop 542, however, other locations are also possible (e.g., along only one side of the cutting loop 542).

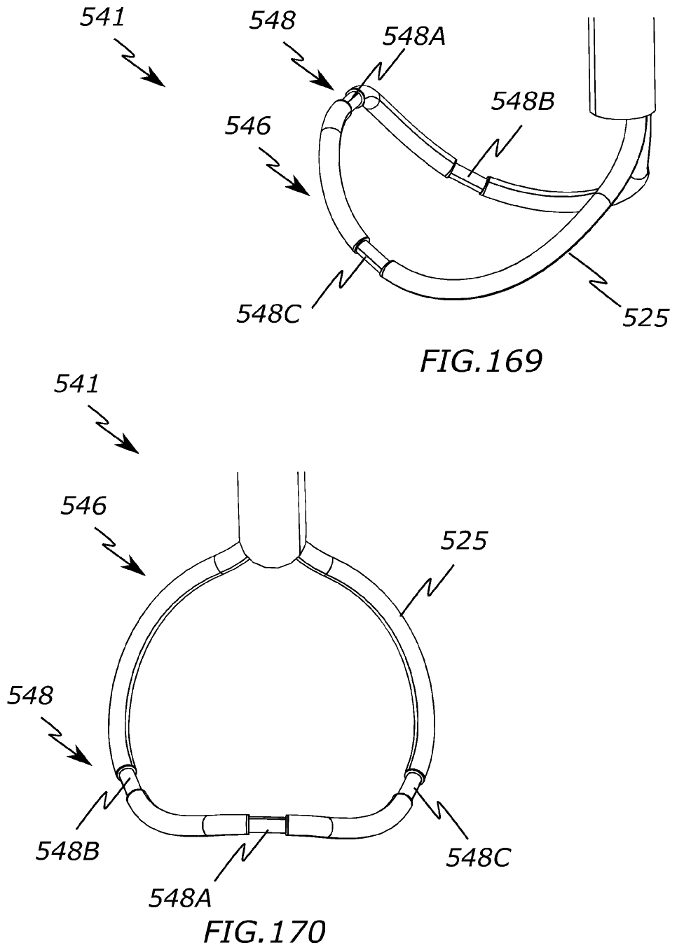

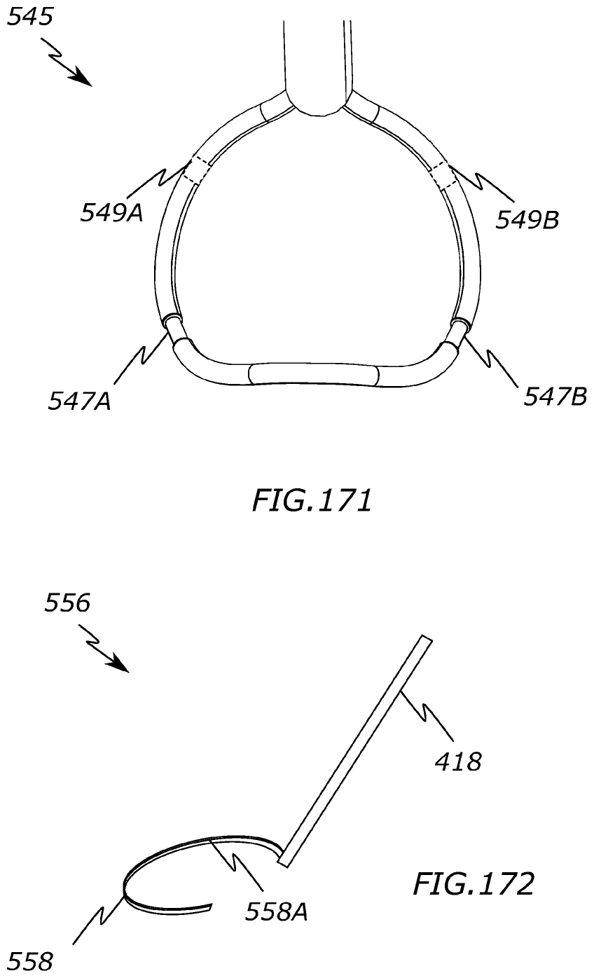

As previously described in this specification, a cutting loop may also include a plurality of electrodes. For example, FIGS. 168-170 illustrate a cutting loop catheter 541 with a distal cutting loop 546 having a plurality of electrodes 548 (e.g., of any shape or configuration mentioned in this specification). In this example, a first electrode 548A is positioned at a middle and distal-most location on the cutting loop 542, while a second electrode 548B and third electrode 548C are located on either side of the first electrode 548A and within a distal half of the cutting loop 542. FIG. 171 illustrates a similar cutting loop 545 without a middle electrode and having a first electrode 547A and second electrode 547B on either lateral side of the distal half of the cutting loop 545. Element numbers 549A and 549B illustrate locations for additional optional electrodes or alternative positions for the electrodes.

Monopolar RF may be used to provide power for these cutting loops with multiple electrodes (as well as with single electrode embodiments). In such examples, each of the multiple electrodes may be connected to the same electrical circuit so that they can be activated simultaneously. For example, each electrode may be connected to an underlying shape memory wire of the cutting loop that is further electrically connected to the inner control member (or a separate electrical wire) that is ultimately connected to an RF power source near the proximal end of the cutting loop catheter.

Alternatively, all or some of the electrodes may be on separate circuits from each other such that they can be independently activated by the RF power source. Hence, additional electrical wires may be included under the insulation layer 525 and connected to one or more of the electrodes. As described later in this specification, this may allow the user, via the RF power source, to activate some or all of the electrodes. Additionally, the electrode impedance may be measured by the RF power source to help determine if the electrodes are in contact with tissue or with blood.

While the cutting loops described in this specification are primarily contemplated as closed loop shapes, other shapes are also possible. For example, open loop shapes, partial spiral shapes, and similar shapes may also be possible. FIG. 172 illustrates one example of a cutting loop device 556 having a hook, a “C” shape, or an open loop shape 558. The curved shape may lie substantially within a single plane or may spiral in a non-planar shape. The inner surface 558A may include one or more electrodes, the outer surface may include one or more electrodes, or electrodes may encompass both inner and outer surfaces. This shape may be particularly helpful for use in cutting chordae, in addition to tissue freeing a heart valve therapy device.

Whether a full loop shape is used, the prior hook shape, or other chordae cutting devices described in this specification are used, several different approaches to reach and cut the chordae of a mitral valve are possible. For example, a transaortic approach to the mitral valve may be possible. A trans-apical approach to the mitral valve may also be possible. A trans-atrial approach to the mitral valve is also possible. A snare and/or capture basket may optionally be used after the desired cutting has been performed.

The cutting loops of this specification, including those most recently discussed in FIGS. 156-168, may be included as a single catheter device with a capture basket, such as that seen in FIG. 9-11 or 105 among other figures, or may be a standalone cutting loop catheter that is connected to an inner control member (e.g., a control wire) within a sheath, similar to that described for the snare catheters of this specification. The later configuration may allow a user to use different cutting loop configurations for a procedure and/or use the cutting loop catheter separately from the capture basket catheter (i.e., a cutting loop catheter, a capture basket catheter, and/or a snare catheter may be each be individually used as needed).

Additionally, it is contemplated that some procedures may only necessitate the use of a cutting loop or chordae cutting tool, and therefore such a “stand-alone” cutting loop catheter may be used in such cases. For example, a cutting loop of a cutting catheter may be used as both a cutting loop and as a snare loop. The cutting loop may be advanced into a patient's heart and the desired tissue (chordae and/or leaflet tissue is cut). The cutting loop is placed around a heart valve therapy device (e.g., valve clip) and tightened, while the electrodes of the cutting loop may optionally be activated again to cut any remaining tissue necessary to completely free the heart valve therapy device. The cutting loop may then be used to withdraw the heart valve therapy device.

The cutting loops and similar electrical cutting mechanisms of this specification may include one or more electrodes that form a circuit connectable to an RF power source. In the environment of the heart, such electrodes may be in contact with blood and tissue to varying degrees, which can complicate tissue cutting. The Applicants have found that it is sometimes necessary for electrodes on a cutting loop or similar device to create a spark or plasma within the heart to desirably cut tissue. The Applicants have also found that by using RF power within a certain range, specifically within an inclusive range of about 5 watts to 1600 watts, and by using electrodes with certain areas, creation of such plasma and subsequent cutting of tissue may be enhanced. Note that the use of additional electrodes on the same electrical circuit may require proportionally more power (or individual power generators for each electrode), while individually activating only a specific electrode of a plurality of electrodes that are all on different electrical circuits may limit the needed power.

For example, a single electrode may have a surface area within an inclusive range of about 0.003 square inch to about 0.006 square inch. In another example, a single electrode may have a surface area within an inclusive range of about 0.003 square inch and 0.030 square inch. In another example, a single electrode may have a surface area within an inclusive range of about 0.004 square inch to about 0.005 square inch. In one specific example, a single electrode may include a surface area of about 0.00437506 square inch. Cutting loops with multiple electrodes may have similar values for each electrode that is part of a cutting loop (e.g., 0.00437506 multiplied by the number of electrodes). The electrode area may include all exposed surface, including horizontal and vertical surface areas.

The present specification describes different capture baskets used to capture and remove a heart valve therapy device. As previously discussed, a patient's heartbeat may cause movement of the valve leaflets, chordae, and the heart valve therapy device, among others. This movement can cause capture baskets (as well as other components) to move or bounce around within the patient's heart. Hence, it may be helpful to limit the degrees of freedom of movement of the capture basket in its deployed or expanded state.

For example, it may be helpful to provide multiple points of connection along the length of the basket to the basket catheter (e.g., the tip of the basket catheter). In a more specific example, the catheter may connect near a first distal most location (e.g., a cinching loop) and at a second location further distal (e.g., to portions of wire further distal on the basket). Alternatively, the exit openings for the cinching loop of the basket may be located proximal to the tip of the catheter so as to allow the tip of the catheter to extend distally alongside the basket (e.g., 2-10 mm) and thereby acting as a backstop to prevent the basket from rotating around the tip of the catheter.

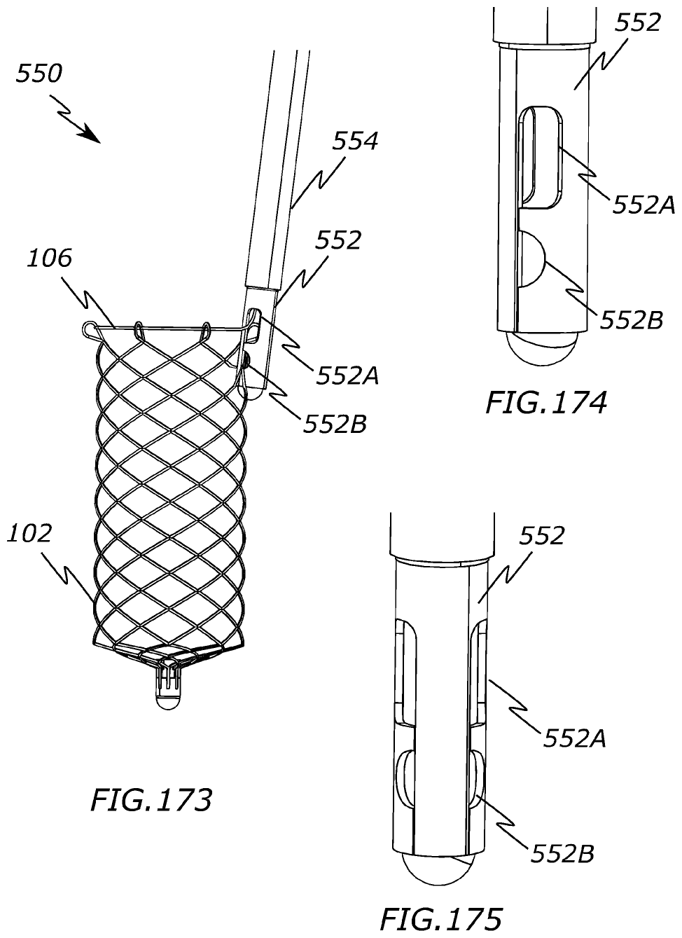

For example, FIGS. 173-175 illustrate various aspects of a capture basket catheter 550 with multiple connection points to a capture basket 102. The present capture basket catheter 500 is depicted as only including a capture basket 102 and can be used with other components described in this specification, such as a snare catheter and a cutting loop catheter. However, a cutting loop may also be incorporated into the catheter 550 as described in other embodiments in this specification.

The capture basket catheter 550 may include an elongated catheter body having at least one internal lumen through which the wire of the cinching loop 106 is positioned through, allowing a user at the proximal end of the catheter 550 to pull and cinch the top of the basket 102 closed.

The distal portion of the catheter 550 may include a distal tip member 552 that helps connect the basket to the catheter 550 at two axial or longitudinal positions. Specifically, the distal tip member 554 may include one or more (e.g., 2) openings 552A at a first axial location (i.e., at a first length from a distal end of the distal tip member 552A). The wire of the cinching loop 106 may pass through the one or more openings 552A and into the lumen of the catheter body 554 (e.g., if two openings are present, one portion of the wire may pass into one opening and a second portion of the wire may pass into a second opening).

The distal tip member 554 may also include a second connection point that is located distally of the openings 552A. The second connection point may be one or more openings 552B (e.g., 2) through which wires of the basket 102 pass through or alternatively a separate connection member, such as a wire or tie, may be connected, tied, welded, adhered, or otherwise connected to both a portion of the basket 102 and the to the distal tip member 554 through the openings 552B. Alternatively, the distal tip member 554 may be directly welded or adhered to wires of the basket 102 or the further distal portion of the distal tip member 554 may not be further connected and may instead act as a backstop to prevent rotation of the basket beyond the axis of the basket catheter 550.

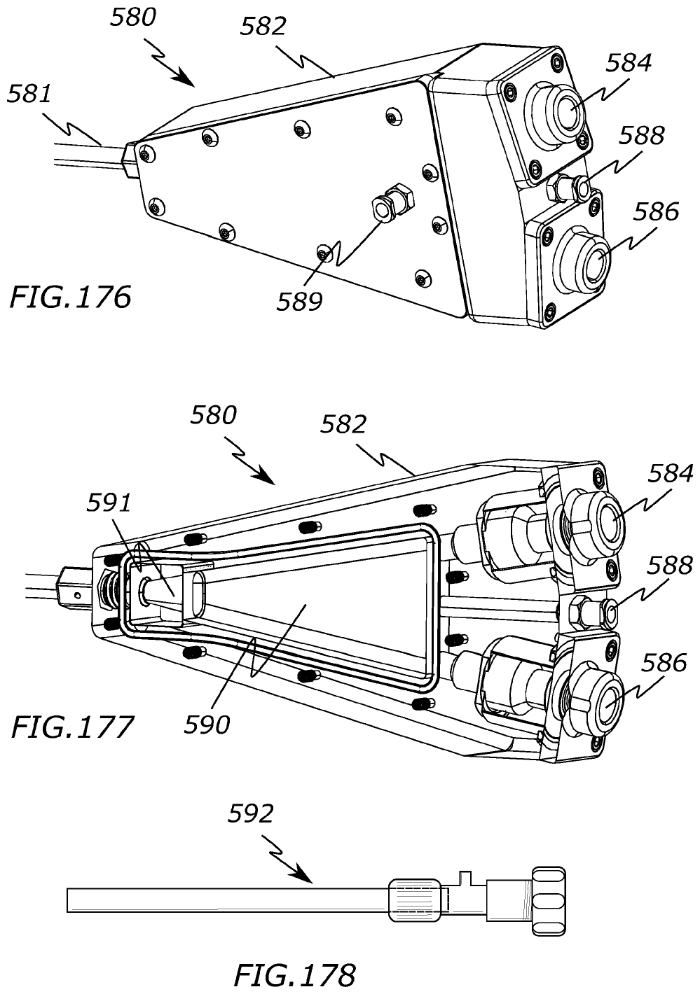

As previously described, it may be desirable to use individual catheters for the cutting loop, snare loop, and the capture basket in some circumstances, or catheters with multiple components on a single catheter (e.g., a cutting loop and a capture basket). In that respect, FIGS. 176-179 illustrate a delivery sheath 580 that assists the user with delivering and/or switching between multiple catheter devices. Generally, the delivery sheath may include an elongated tubular portion 581 having a sheath lumen that is connected to a hub 582. The hub 582 acts as an entrance to the elongated tubular portion 581 for multiple catheters during a procedure.

The hub 582 may include a plurality of ports that open into an interior cavity 590 formed by the housing of the hub 582. The hub 582 may include 2, 3, 4, 5, 6, or more ports. In the present example, the hub 582 may include three ports 584, 588, and 586 that open at the proximal end of the hub 582 at various angles relative to each other.

The cavity 590 may taper or decrease in width in the distal direction toward the elongated tubular portion 581 to allow multiple catheters to converge and all enter the lumen of the elongated tubular portion 581. In one example, the cavity 590 may have a generally triangular shape. To further assist the convergence into the elongated tubular portion 581, a funnel component 591 may be included at a distal end of the cavity 590. The funnel component 591 may have a passage that decreases in width but also has a generally oval cross-sectional shape, the width of which generally aligns with the triangular width of the cavity 590. The funnel component 591 may be a separate component from the housing components forming the cavity 590 or may be an integral shape of the housing components forming the cavity 590.

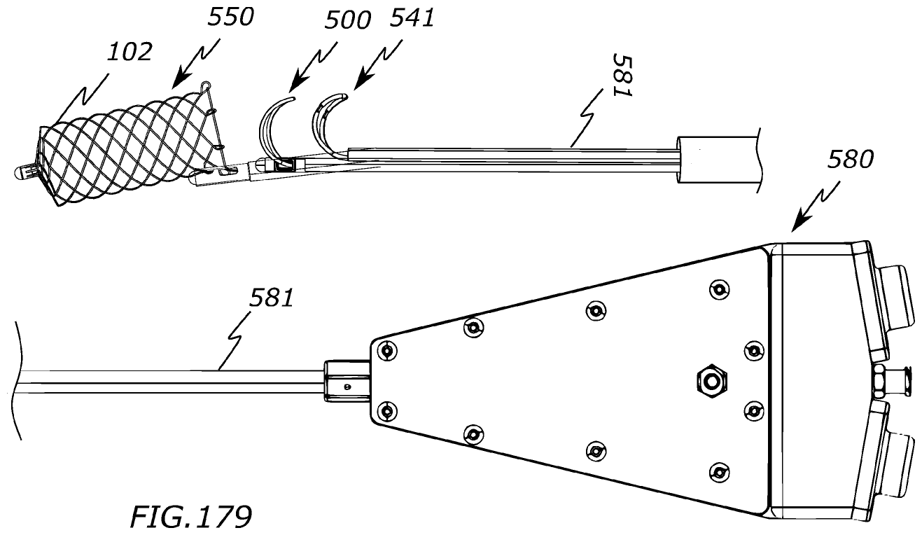

An additional port 589 (FIG. 176) may also be included to access the cavity 590 for purposes of removing air and/or connecting a fluid drip line during a procedure. Note that port 589 is positioned such that any air within the cavity 590 may rise toward the port 589 when the port is facing upwards to facilitate air removal. Any or all of the ports may include a valve, such as a hemostasis valve for closing off the ports when not in use or around a catheter that is passed through. FIG. 179 illustrates the delivery sheath in use with three individual catheters 550, 500, and 541, which were previously described in this specification.

It may also be desirable to use a loading tool, such as the loading tool 592 in FIG. 178 to load a catheter or guidewire into the hub 582, and particularly to facilitate exchanging such tools within the patient during a procedure. In one example, the loading tool 592 may have an elongated tubular portion that is optionally tapered at its distal end and may further include a valve, such as a hemostasis valve, at its proximal end. Some ports, such as ports 584 and 586 may be relatively larger than others to accommodate the larger diameter loading tool during a procedure, while other ports, such as port 588 is relatively smaller and may not be intended for use with the loading tool or exchanges during a procedure (e.g., may have a locking Tuohy Borst valve). The loading tool 592 may also include a releasable locking mechanism that engages the catheter within it and therefore may maintain the catheter's position in the patient during a procedure.

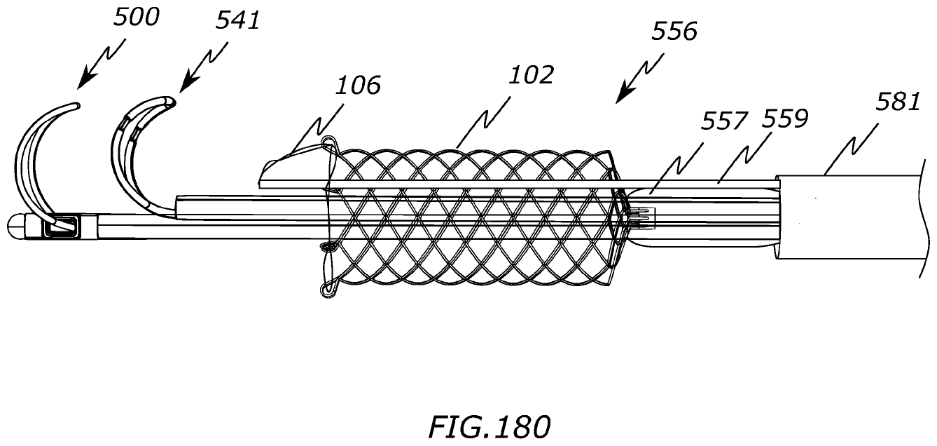

The capture basket catheter has been primarily previously described as being oriented such that it opens proximally relative to the user and catheter. However, depending on the approach into a patient's heart, it may be desirable to have a distally opening basket. For example, FIG. 180 illustrates a capture basket catheter 556 with a basket 102 that opens distally. A proximal end of the basket 102 may be connected to an elongated catheter or sheath 557 and the snare catheter 500 and the cutting loop catheter 541 may be positioned alongside or through a center channel of the capture basket catheter 556. In either arrangement, features may be included to reduce the likelihood of scraping off any electrical insulation on the snare catheter and/or cutting catheter (e.g., the basket 102 may include a coating to reduce abrasion and/or sharp edges rounded/reduced). Optionally, the basket 102 may also include a cinching loop 106 to close the basket 102, similar to those previously discussed in this specification. Such a cinching loop 106 may pass through a second catheter 559 or portion of the catheter 557 that has a distal end extending distally of the distal opening of the basket 102.

Chordae, or chordae tendineae, are typically considered inelastic cords of fibrous connective tissue that connect the papillary muscles to the tricuspid valve and the mitral valve in the heart. Multiple chordae attach to each leaflet or cusp of the valves. During a procedure to remove a heart valve therapy or other procedures, it may be desirable to move, restrict, and/or cut one or more chordae. In that regard, a chordae capture and cutting device may be helpful during some procedures.

One general example of a chordae capture and cutting device may include an elongated catheter body with at least one lumen extending through and a capture member that is movable into and out of a distal opening into the lumen of the catheter body. A distal end of the capture member may include a feature or shape that can engage one or more chordae within a heart. For example, the capture member may be an elongated shape memory wire having a distal end with a memory shape of a spiral, loop, hook, conical/helical loops, nautical shapes, square loops, oval loops, circular loops, triangular loops, rectangular loops and similar shape when unconstrained. Alternatively, the capture member may steerable catheter or guidewire that allows a user to actuate and create with previously described shapes when desired. Once engaged with one or more chordae, the capture member may either restrict the position of the chordae or may cut the chordae. For example, the chordae may be cut by pulling them partially into the opening and lumen of the catheter and advancing a cutting element with a sharp edge against the chordae or by activating an RF electrode on the capture member.

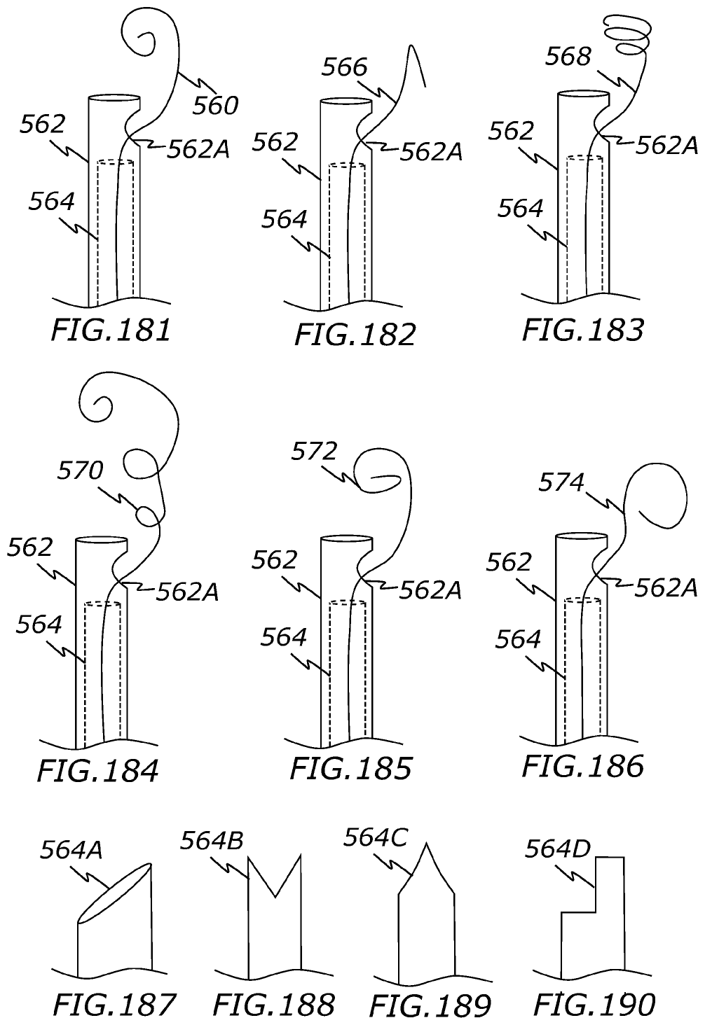

FIG. 181 illustrates one example of a chordae capture and cutting device comprising an elongated catheter body 562 having at least one lumen the opens at a proximal end of the device and at an opening 562A at a distal end of the device. The opening 562A is shown opening at a sidewall of the catheter body 562 (e.g., generally perpendicular relative to an axis of the catheter body 562), but may also be located at the very distal tip in an axial or distally opening position.

A cutting element 564 may be positioned within the lumen of the catheter body 562. In the present example, the cutting element may have a generally tubular shape at its distal end and may extend to a proximal end of the catheter body so that the user may translate the cutting element 564 distally and proximally. A distal edge of the cutting element 564 has a sharpened edge, here the circular terminal edge of the tubular shape, that may be used to cut one or more chordae as described further below.

A capture member 560 may be an elongated wire, plurality of wires, tubular structure, or similar elongated structure, and may be positioned within the lumen of the catheter body 562 and either adjacent to the cutting element 564 or within a lumen of the cutting element 564 as seen in FIG. 181. In the present example, the distal end of the capture member 560 has a curved or spiral shape (e.g., either a spiral within a single plane or a helical/spiral shape) when unconstrained (memorized shape imparted to shape memory material).

The capture member may have a variety of different distal end shapes for capturing one or more chordae (e.g., that form a curved diameter within an inclusive range of about 0.15 inches to about 1.5 inches). For example, FIG. 182 illustrates a capture member 566 has a hook shape. In another example, FIG. 183 illustrates a capture member 568 forming a plurality of loops in a helical shape. In another example, FIG. 184 illustrates a capture member 570 forming a plurality of loops and terminating with a spiral shape. In another example, FIG. 185 illustrates a capture member 572 with a spiral shape that terminates with an abrupt curve or hook shape. FIG. 186 illustrates a capture member 574 that forms a relatively wide, gentle curve. The underlying wire, wires, or structure of the capture member may have a diameter with an inclusive range of about 0.001 inch to 0.030 inch).

The cutting element 564 is illustrated as a tubular shape with a generally perpendicular distal edge relative to an axis of the cutting element 564. However, other shapes and configurations are possible. For example, FIG. 187 illustrates a bias cut edge 564A in which the edge is positioned at a non-perpendicular angle (e.g., 45 degrees) relative to an axis of the cutting element 564. In another example, FIG. 188 illustrates a distal edge 564B forming a plurality of points (e.g., two points for a forked shape). In another example, FIG. 189 illustrates a distal edge forming a single point 164C. In another example, FIG. 190 illustrates a distal edge 564D forming a stepped shape with square or relatively perpendicular edges. Any of these distal edges may be created in the form of a tube or a solid device (e.g., edge 564C may be a conical shape).

Any of the cutting element examples may be used with any of the capture member examples disclosed in this specification.

FIGS. 191-194 illustrate an example method of use of a chordae capture and cutting device. First, a distal end of the catheter body 562 is advanced into the heart such that the opening 562A is positioned near the chordae 21 to be cut, as seen in FIG. 191. In the present example, this may involve accessing the left atrium and passing through the leaflets 22, 24 of the valve, though accessing the left atrium is also possible.

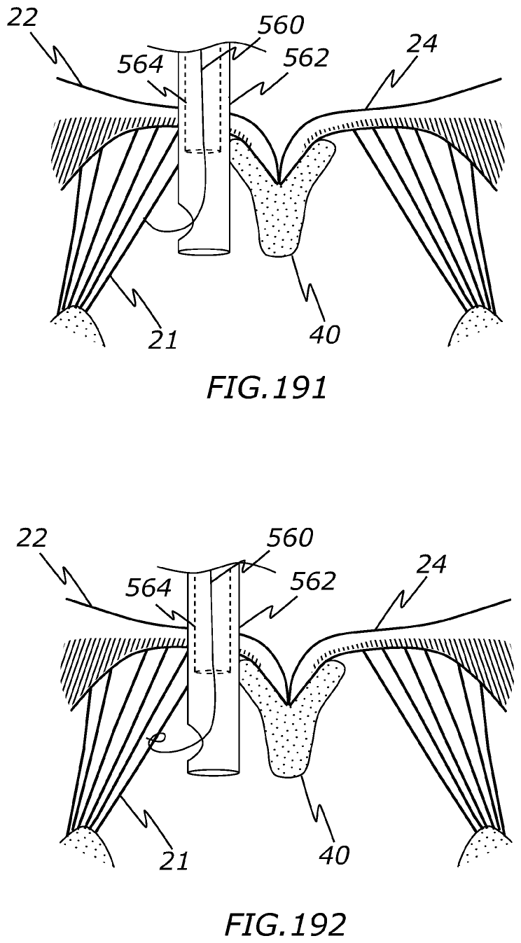

As seen in FIG. 192, the capture member 560 is advanced distally out of the opening 562A, which allows the distal portion of the capture member 560 to slowly assume its unconstrained, memorized shape as it advances, which is a spiral or loop in this example. In this example, as the distal portion of the capture member 560 is distally advanced, it will slowly take on its memorized shape to partially or fully surround one or more of the chordae 19. Additionally, the capture member 560 can be rotated, particularly if it has a helical shape, to surround chordae 21 one or more times.

As seen in FIG. 193, once the capture member 560 has surrounded one or more chordae with its distal end, the capture member 560 can be proximally withdrawn, pulling at least some portion of the chordae into the lumen of the catheter body 562. In that respect, it may be helpful that the opening 562A is large enough to allow the distal end of the capture member 560 back into the lumen of the catheter body 562 after it has expanded without releasing the chordae 19.

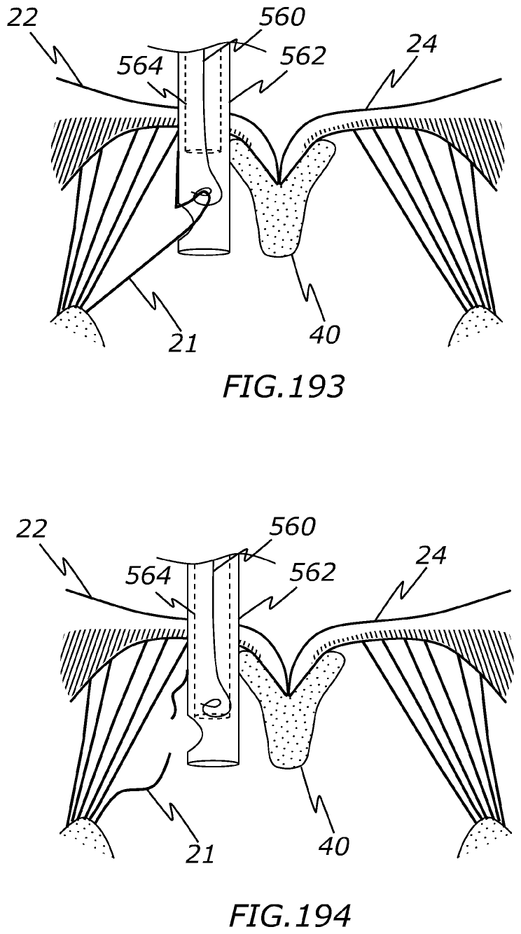

Turning to FIG. 194, the cutting element 560 may be distally advanced by the user so that its distal cutting edge contacts and cuts the chordae 19. The previously described positioning and cutting element can be performed multiple times during a procedure until all of the target chordae 21 have been cut.

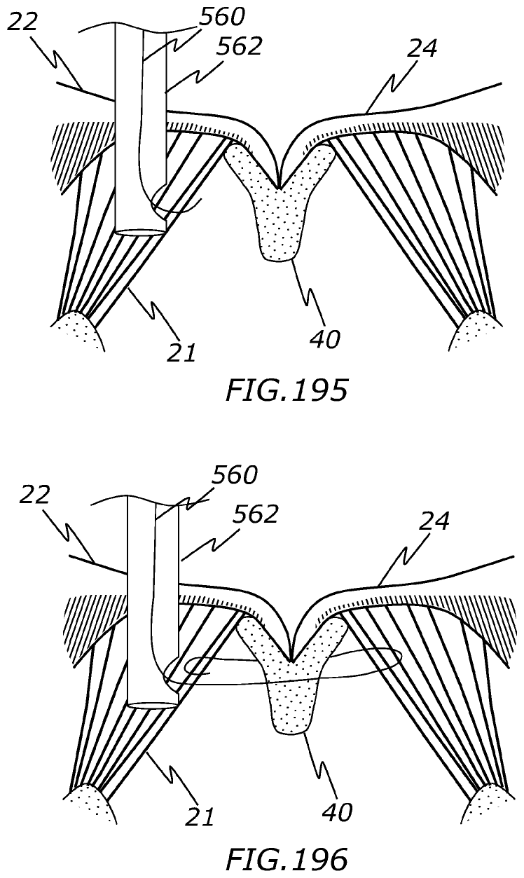

FIGS. 195-198 illustrate a similar technique except that the capture member 560 is initially distally advanced to encompass both chordae 21 and a heart valve clip 40 (FIG. 196), tightened (FIG. 197), and then the capture member 560 is moved downward off of the heart valve clip 40 (FIG. 198). The chordae 21 may be cut with a cutting element as previously described or may be cut with a separate cutting device (e.g., a hook shaped cutting loop).

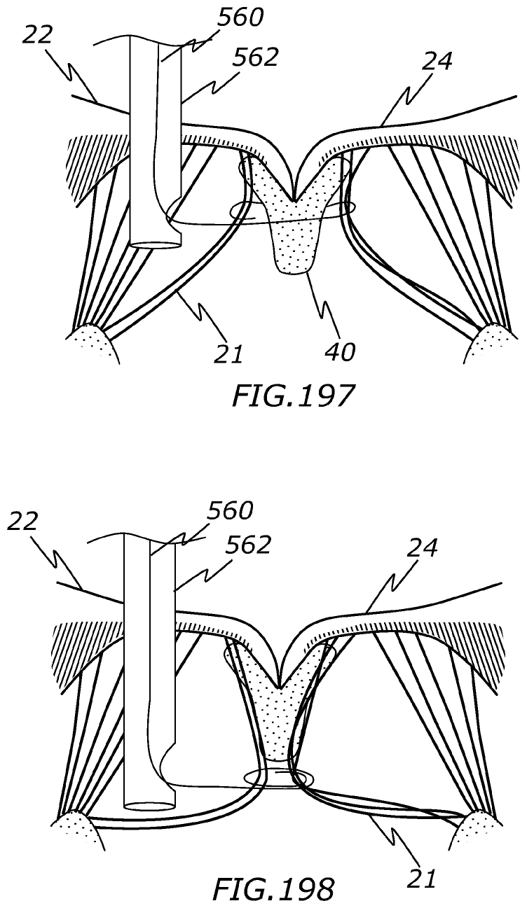

FIGS. 199-200 illustrate a similar procedure as previously described, however, the capture member 560 may include an electrode 561 that may be energized (e.g., with RF energy) to cut the chordae.

As previously discussed in this specification, any of the cutting loops disclosed in this specification may include a plurality of electrodes that are independently wired (i.e., that each form different circuits that can be independently actuated). For example, the cutting loop 546 in FIGS. 168-170. Such a multi-electrode cutting loop may be connected to a power source (e.g., an RF power source) that can independently sense and energize these electrodes, allowing a user to determine if a specific electrode is in contact with tissue or blood, and then specify which of the electrodes should be electrically activated.

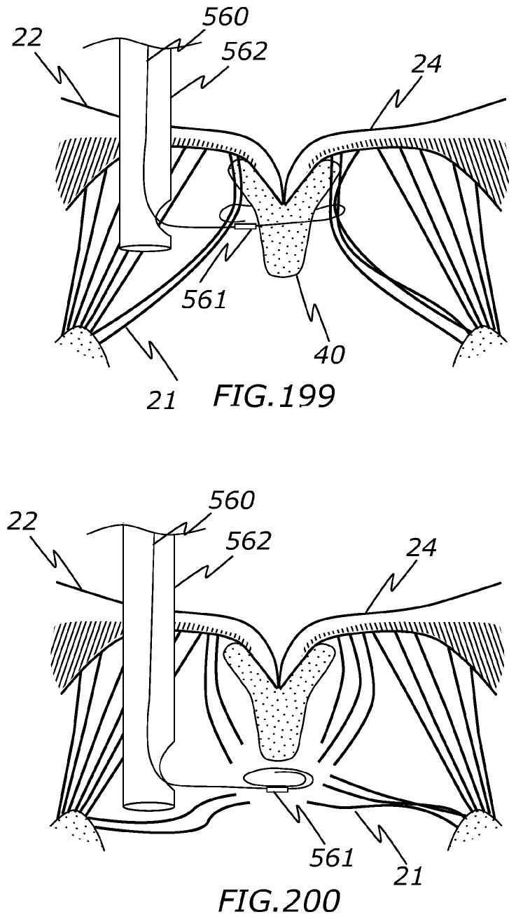

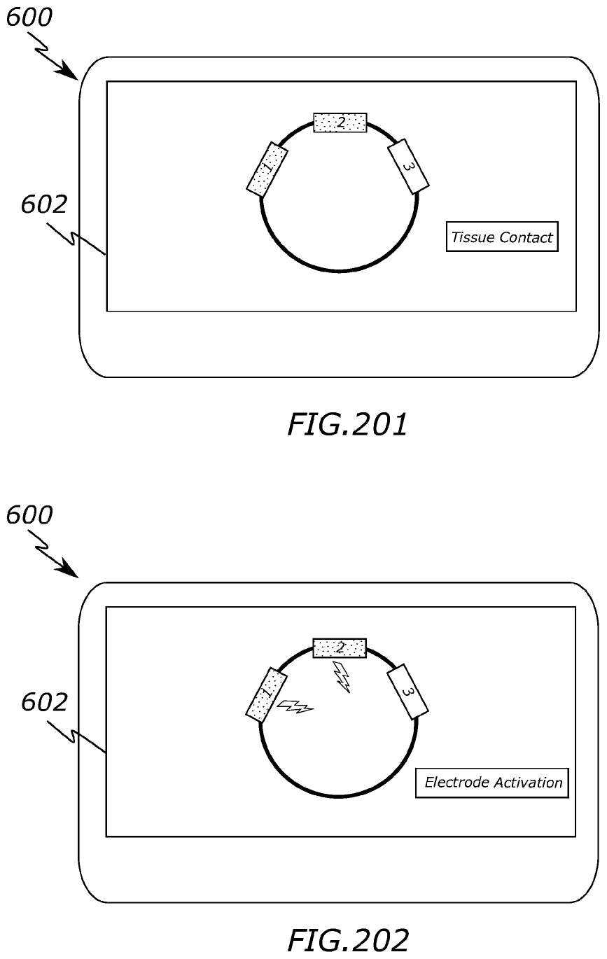

For example, FIGS. 201 and 202 illustrate an RF power generator or source 600 that has a user interface 602 configured to display information either within a housing of the RF power generator or as a separately connectable display. The generator 600 may also include user inputs, such as a keyboard or via a touch screen interface. It should be understood that the generator 600 is a computerized device that may include a processor and memory in which software is stored which can be executed by the processor to perform the described functionality.

The user interface 602 may include a first tissue contact display that displays information indicating which of the electrodes is in contact with tissue (e.g., electrodes 1, 2, and/or 3), as seen in FIG. 201. The tissue contact display may be in the form of a graphical depiction of a cutting loop as seen in FIG. 201, numerical indications, or variations thereof. Tissue contact may be determined by continuously measuring electrical values for each electrode, such as impedance and when the electrical value exceeds a certain threshold, tissue contact would be determined.

The user interface 602 may also include a second electrode activation display that allows a user to determine which electrodes will be electrically active when the power is delivered to the cutting loop, as seen in FIG. 202. The electrode activation display may be in the form of a graphical depiction of a cutting loop as seen in FIG. 202, numerical indications, or variations thereof. They display may also include indicia, symbols, words, or other display elements that indicate if an electrode is active or deactivated. Additionally, the activation display and the tissue contact display may be incorporated into the same screen so as to show data at the same time.

Since some circumstances require the removal of a heart valve clip from a patient, implanting a heart valve clip that is more easily removable may be desirable. For example, a heart valve clip may include a shape or region that can be more easily snared, such as a groove or enlarged distal end. Additionally, a heart valve clip may be configured to apply RF energy at specific locations contacting valve tissue so that, when contacted with an electrode from a cutting loop, the RF energy is directed to those areas of tissue contact to more quickly and reliably free the heart valve clip. For example, the heart valve clip may be composed of an electrically conductive material (e.g., entirely or electrical pathways) and the remaining portions insulated except for areas within the “arms” of the clip that engage the tissue. Alternatively, the outside surface of the heart valve may have electrode areas (e.g., raised areas, ridges, or similar features) that form electrodes, allowing a user to apply power to the valve clip and then turn, twist, pull, or otherwise move the valve clip around to cut and free it from patient.

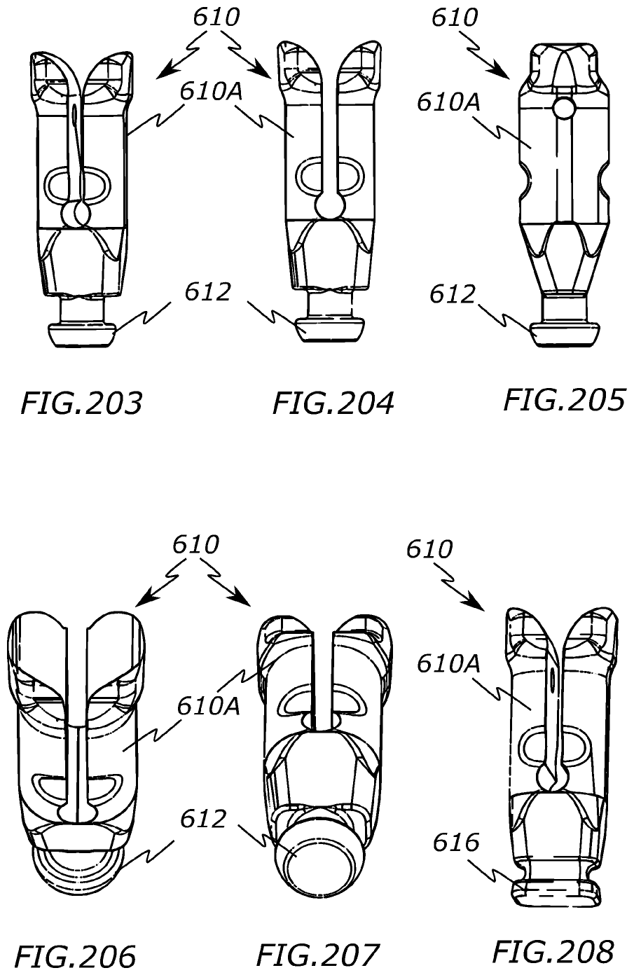

FIGS. 203-207 illustrate one example of a heart valve clip 610 that that has an elongated body with a leaflet connection mechanism having two elongated arms 610A that form a “U” shape that can be adjusted to pinch or engage leaflet tissue. The distal end of the valve clip 610 may include a snare engagement feature 612 that allows easier engagement of a snare loop. In the present example, the feature 612 may include a channel or groove extending partially or fully around the diameter of the valve clip 610. Alternatively, the valve clip 610 may include an enlargement on its distal end. In either case, a snare loop may be positioned over the feature 612 and tightened to create a firm connection. The distal end of the feature 612 is depicted as circular or cylindrical, but other shapes are possible, such as the more rectangular shape of feature 616 in FIG. 208.

The heart valve 610 may include areas that form an electrode, such as on the inner surface of the arms 610A. These areas may include raised edges or shapes similar to previously discussed electrodes of a cutting loop. Depending on the construction of the valve clip 610, an insulative coating may be applied to areas unlikely to contact tissue.

A cutting loop may contact another location on the valve clip 610 that is in electrical communication with the electrode surfaces, allowing those electrode surfaces to conduct electrical current. For example, the feature 612 may create an electrical path to the inner surfaces of the arms 610A which are in contact with leaflet tissue. In such an example, a cutting loop with electrodes may act as a snare to encompass and engage the feature 612, while also delivering power to various areas of the valve clip 610 that is engaging leaflet tissue.

The present specification and drawings include many different embodiments and features of removal devices and methods of use thereof. While features or techniques may be depicted in connection with a specific embodiment, it is the intent of the Applicant that any features shown in any of the embodiments can be incorporated in other embodiments. Put another way, any of the features described herein can be mixed and matched with each other and the claims should therefore not be otherwise limited or otherwise restricted to only the embodiments discussed and depicted herein.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an ingredient or element may still actually contain such item as long as there is generally no measurable effect thereof.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description should be read to include one or at least one and the singular also includes the plural unless it is obvious that it is meant otherwise.

Furthermore, the figures depict preferred embodiments for purposes of illustration only. One skilled in the art will readily recognize from the discussion herein that alternative embodiments of the structures and methods illustrated herein may be employed without departing from the principles described herein.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A snare catheter for use in medical treatment of a patient, comprising:
an elongated sheath having a lumen and one or more openings at a distal portion of the elongated sheath into the lumen;
an inner control member extending within the elongated sheath;
a snare loop at a distal end of the inner control member and extending out of the one or more openings; and,
electrical insulation configured to reduce transfer of electrical current between an electrical cutting device within a heart of a patient.

2. The snare catheter of claim 1, wherein the electrical insulation is disposed over some or all of the snare loop.

3. The snare catheter of claim 1, wherein the electrical insulation comprises a coating layer, or tube comprising silicone, polyolefin, polyimide, dielectric material, PTFE, FEP.

4. The snare catheter of claim 1, wherein the snare loop comprises one or more wires comprising nitinol or stainless steel.

5. The snare catheter of claim 1, wherein the elongated sheath further comprises a distal tip member located at a distal end of the elongated sheath and forming the one or more openings; and wherein the electrical insulation further comprises a layer, coating, or structural material.

6. The snare catheter of claim 5, wherein the distal tip member comprises rounded, curved, or blunted shapes around a perimeter of the one or more openings to reduce or limit damage to the electrical insulation.

7. The snare catheter of claim 6, wherein the rounded, curved, or blunted shapes are formed by an insert connected to the distal tip member or are formed by the distal tip member.

8. The snare catheter of claim 7, wherein the one or more openings is located on a side of the distal tip member relative to an axis of the elongated sheath.

9. A snare catheter for use in medical treatment of a patient, comprising:
an elongated sheath having a lumen;
an inner control member extending within the elongated sheath;
a snare loop at a distal end of the inner control member; and,
a distal tip member connected at a distal end of the elongated sheath, the distal tip member having a first opening and a second opening into the lumen of the elongated sheath; wherein the snare loop is positioned through the first opening and the second opening such that a structural feature in between the first opening and the second opening prevent the snare loop from being completely pulled into the lumen of the elongated sheath.

10. The snare catheter of claim 9, wherein the structural feature of the distal tip member is a bar or a pin.

11. A snare catheter for use in medical treatment of a patient, comprising:
an elongated sheath having a lumen and one or more openings at a distal portion of the elongated sheath into the lumen;
an inner control member extending within the elongated sheath; and,
a snare loop at a distal end of the inner control member and extending out of the one or more openings;
wherein the snare loop is configured to limit deflection of a distal tip of the snare loop to an inclusive range of about 0 mm to about 10 mm when force is applied to the snare loop within an inclusive range of about 0 grams to about 3 grams.

12. The snare catheter of claim 11, wherein the snare loop is configured to limit deflection of the distal tip of snare loop to an inclusive range of about 0 mm to about 5 mm when force is applied to the snare loop within an inclusive range of about 0 grams to about 2 grams.

13. The snare catheter of claim 11, wherein the snare loop comprises a solid shape memory wire having a diameter within an inclusive range of about 0.001 inch to 0.03 inch.

14-58. (canceled)
